(19) United States
(12) Patent Application Publication
(10) Pub. No.: US 2024/0424246 A1
(43) Pub. Date: Dec. 26, 2024

(54) SYSTEMS AND METHODS TO INDUCE SLEEP AND OTHER CHANGES IN USER STATES

(71) Applicant: INTERAXON INC., Toronto (CA)

(72) Inventors: Christopher Allen AIMONE, Scarborough (CA); Walter John GREENLEAF, Portola Valley, CA (US)

(21) Appl. No.: 18/699,561

(22) PCT Filed: Oct. 11, 2022
(86) PCT No.: PCT/CA2022/051495

§ 371 Date: Apr. 8, 2024

Related U.S. Application Data
(60) Provisional application No. 63/254,028, filed on Oct. 8, 2021.

(51) Int. Cl. A61M 21/02; A61B 5/16; A61M 21/00; G16H 20/30; G16H 40/63; G16H 50/20; G16H 50/70

(52) U.S. Cl. CPC A61M 21/02; A61B 5/165; G16H 20/30; G16H 40/63; G16H 50/20; G16H 50/70; A61M 2021/0016; A61M 2021/0022; A61M 2021/0027; A61M 2021/0072; A61M 2021/0077; A61M 2205/332; A61M 2205/3375; A61M 2230/06; A61M 2230/10; A61M 2230/14; A61M 2230/40; A61M 2230/50; A61M 2230/63; A61M 2230/65

(57) ABSTRACT

In accordance with an aspect, there is provided a computer system for achieving a target user state by modifying content elements provided to the at least one user. The system includes at least one computing device in communication with at least one bio-signal sensor and at least one user effector, the at least one bio-signal sensor can be configured to measure bio-signals of at least one user, the at least one user effector can be configured to provide content to the at least one user, wherein the content comprises one or more content elements. The at least one computing device can be configured to provide the content to the at least one user via the at least one user effector, compute a difference between the user state of the at least one user before an interval and the target user state using the bio-signals of the at least one user, modify one or more of the content elements provided to the at least one user during the interval based on the difference between the user state of the at least one user before the interval and the target user state, compute a difference between the user state of the at least one user after the interval and the target user state using the bio-signals of the at least one user, modify one or more of the content elements provided to the at least one user after the interval based on the difference between the user state of the at least one user after the interval and the target user state.

### CROSS-REFERENCE

This application claims all benefit including priority to U.S. Provisional Patent Application 63/254,028, filed 8 Oct. 2021, and entitled “SYSTEMS AND METHODS TO INDUCE SLEEP AND OTHER CHANGES IN MENTAL STATES”, the entire contents of which are hereby incorporated by reference herein.

### FIELD

Embodiments of the present disclosure generally relate to the field of brain state guidance, and more specifically, embodiments relate to devices, systems and methods for improved content delivery to induce a state in a user.

### BACKGROUND

When an individual is trying to go to sleep, they may need to bring their mind from an active and alert state, to a relaxed state, and finally into a sleep state. In an effort to relax, some individuals may use white noise machines, audio programs, or music at a low volume. This sort of stimulus can provide individuals with sufficient engagement to occupy a busy mind and bring it to a relaxed state. This level of engagement may be helpful to relax, but it may become detrimental to bringing a user into a sleep state. The volume may be too loud or the content may be too engaging. Similar problems may arise when attempting to bring about other user state changes.

A system may turn off using a timer, but that offers no guarantee that the individual will be asleep when the system shuts down. A system may remove a stimulus when the user is asleep but this may rouse the user and interfere with their sleep.

There exists a need for systems that use the internal user states (e.g., brain states) to assist a user in achieving a state change, or at least alternatives. There exists a need for systems that may adapt and change the presentation of content to permit users to engage with or disengage with the content as needed to change states (e.g., fall asleep). There exists a need for systems with improved and enhanced efficacy in a sleeping aide.

### SUMMARY

Systems, methods, and devices described herein provide an improved or alternative mode of guiding a user to an ultimate user state (e.g., a sleep state). In some embodiments, the systems, methods, and embodiments can detect a user's state and modify content to bring the user to the ultimate user state. For example, some systems can detect when a user is on the edge of sleep and cut the content to bring the user into a sleep state. These systems are principally directed at inducing sleep states, however the systems, methods and devices described herein may be effective at inducing other states as well (e.g., flow states, wakefulness states, fear states, alert states, altered states, etc.).

In accordance with an aspect, there is provided a computer system for achieving a target user state by modifying content elements provided to at least one user. The system includes at least one computing device in communication with at least one bio-signal sensor and at least one user effector, the at least one bio-signal sensor can be configured to measure bio-signals of at least one user, the at least one user effector can be configured to provide content to the at least one user, wherein the content comprises one or more content elements. The at least one computing device can be configured to provide the content to the at least one user via the at least one user effector, compute a difference between the user state of the at least one user before an interval and the target user state using the bio-signals of the at least one user, modify one or more of the content elements provided to the at least one user during the interval based on the difference between the user state of the at least one user before the interval and the target user state, compute a difference between the user state of the at least one user after the interval and the target user state using the bio-signals of the at least one user, modify one or more of the content elements provided to the at least one user after the interval based on the difference between the user state of the at least one user after the interval and the target user state.

In accordance with a further aspect, computing a difference between the user state of the at least one user before an interval and the target user state comprises determining that a trigger user state has been achieved using the bio-signals of the at least one user.

In accordance with a further aspect, the at least one user effector may be configured to provide content to a plurality of users, and the user state can be based on the bio-signals of each user of the plurality of users.

In accordance with a further aspect, the user state may be determined based in part on a prediction model.

In accordance with a further aspect, the system further comprising a server configured to store the prediction model and provide the prediction model to the at least one computing device. The at least one computing device is configured to update the prediction model based on the difference between the user state of the at least one user after the interval and the target user state.

In accordance with a further aspect, the prediction model comprises a neural network.

In accordance with a further aspect, the prediction model may be based in part on a user profile.

In accordance with a further aspect, the prediction model may be based in part on data from one or more other users.

In accordance with a further aspect, the one or more other users may share a characteristic with the at least one user.

In accordance with a further aspect, the interval may be based in part on a current user state of the at least one user.

In accordance with a further aspect, the interval may be based in part the content. In accordance with a further aspect, the interval is based in part on user input.

In accordance with a further aspect, the target user state may be based in part on the content.

In accordance with a further aspect, the target user state may be based in part on input.

In accordance with a further aspect, the trigger user state may be based in part on the content.

In accordance with a further aspect, the trigger user state may be based in part on input.

In accordance with a further aspect, the modify the one or more of the content elements is based in part on user input.

In accordance with a further aspect, the at least one computing device may be further configured to determine a first user state of the at least one user using the bio-signals of the at least one user, apply a probe modification to one or more of the content elements provided to the at least one user, compute a difference between the first user state of the at least one user and the user state of the at least one user after a probe interval using the bio-signals of the at least one user, and update at least one of the target user state and the trigger user state based on the difference between the first user state and the user state after the probe interval.

In accordance with a further aspect, the at least one computing device is further configured to determine a first user state of the at least one user using the bio-signals of the at least one user before a probe interval, compute a difference between the first user state of the at least one user before the probe interval and a user state of the at least one user after the probe interval using the bio-signals of the at least one user, and update at least one of the target user state and the trigger user state based on the difference between the first user state and the user state after the probe interval.

In accordance with a further aspect, the computing device may be further configured to compute a difference between the user state of the at least one user during the interval and an exit user state using the bio-signals of the at least one user, and modify one or more of the content elements provided to the at least one user based on the difference between the user state of the at least one user and the exit user state.

In accordance with a further aspect, the at least one bio-signal sensor may include at least one of EEG, EOG, EKG, EMG, PPG, heart rate, breath, sweat, gyroscopic, accelerometer, magnetometer, IMU, movement, vibration, sound, pulse wave amplitude, fNIRS, temperature, pressure, and electrodermal conductance sensors.

In accordance with a further aspect, the at least one user effector may include at least one of earphones, speakers, a display, a scent diffuser, heater, climate controller, drug infuser or administrator, electric stimulator, medical device, a system to effect physical or chemical changes in the body, restraints, mechanical device, a vibrotactile device, and a light.

In accordance with a further aspect, the system may further include one or more auxiliary effectors configured to provide stimulus to the at least one user, and the computing device may be further configured to modify the stimulus provided to the at least one user by the auxiliary effector.

In accordance with a further aspect, the modify one or more of the content elements can include transitioning between one or more content samples.

In accordance with a further aspect, the modify one or more of the content elements may include pausing one or more of the content elements.

In accordance with a further aspect, the modify one or more of the content elements comprises pausing one or more of the content elements at time codes associated with natural breaks in the one or more content elements.

In accordance with a further aspect, the computing device is further configured to adjust the interval based on natural breaks in the one or more of the content elements.

In accordance with a further aspect, the content may include at least a first and a second time-coded content sample and the modify one or more of the content elements may include transitioning between a first defined time code of the first time-coded content sample to a second defined time code of the second time-coded content sample.

In accordance with a further aspect, the first defined time code is based on natural pauses in the first time-coded content sample and the second defined time code is based on natural pauses in the second time-coded content sample.

In accordance with a further aspect, the second time-coded content sample is selected from a plurality of time-coded content samples based on at least on of the first time-coded content sample.

In accordance with a further aspect, the selection of the second time-coded content sample is based in part on a prediction model.

In accordance with a further aspect, the content may include time-coded content, and the modify one or more of the content elements may be based in part on a current time code in the time-coded content.

In accordance with a further aspect, the user state may include a brain state.

In accordance with a further aspect, the content elements may have modifications applied at a specific change profile.

In accordance with a further aspect, the trigger user state can include reaching a time code in the content.

In accordance with an aspect, there is provided a method for achieving a target user state by modifying content elements provided to at least one user. The method may include receiving bio-signals of at least one user, providing content to the at least one user, the content comprising one or more content elements, computing a difference between a user state of the at least one user before an interval and the target user state using the bio-signals of the at least one user, modifying one or more of the content elements provided to the at least one user during the interval based on the difference between the user state of the at least one user before the interval and the target user state, computing a difference between the user state of the at least one user after an interval and the target user state using the bio-signals of the at least one user, and modifying one or more of the content elements provided to the at least one user after the interval based on the difference between the user state of the at least one user and the target user state.

In accordance with a further aspect, computing a difference between the user state of the at least one user before an interval and the target user state includes determining that a trigger user state has been achieved using the bio-signals of the at least one user.

In accordance with a further aspect, the providing content to at least one user may include providing content to a plurality of users, the user state may be based on the bio-signals of each user of the plurality of users.

In accordance with a further aspect, the user state may be determined based in part on a prediction model.

In accordance with a further aspect, the method further comprising updating the prediction model based on the difference between the user state of the at least one user after the interval and the target user state.

In accordance with a further aspect, the prediction model comprises a neural network.

In accordance with a further aspect, the prediction model may be based in part on a user profile.

In accordance with a further aspect, the prediction model may be based in part on data from one or more other users.

In accordance with a further aspect, the one or more other users may share a characteristic with the at least one user.

In accordance with a further aspect, the interval may be based in part on a current user state of the at least one user.

In accordance with a further aspect, the interval is based in part the content.

In accordance with a further aspect, the interval is based in part on user input.

In accordance with a further aspect, the target user state may be based in part on the content.

In accordance with a further aspect, the target user state may be based in part on input.

In accordance with a further aspect, the trigger user state may be based in part on content.

In accordance with a further aspect, the trigger user state may be based in part on input.

In accordance with a further aspect, modifying the one or more of the content elements is based in part on user input.

In accordance with a further aspect, the method may further include determining a first user state of the at least one user using the bio-signals of the at least one user, applying a probe modification to one or more of the content elements provided to the at least one user, computing a difference between the first user state of the at least one user and the user state of the at least one user after a probe interval using the bio-signals of the at least one user, updating at least one of the target user state and the trigger user state based on the difference between the first user state and the user state after the probe interval.

In accordance with a further aspect, the method further including determining a first user state of the at least one user using the bio-signals of the at least one user before a probe interval, computing a difference between the first user state of the at least one user before the probe interval and a user state of the at least one user after the probe interval using the bio-signals of the at least one user, updating at least one of the target user state and the trigger user state based on the difference between the first user state and the user state after the probe interval.

In accordance with a further aspect, the method may further include computing a difference between the user state of the at least one user during the interval and an exit user state after using the bio-signals of the at least one user, and modifying one or more of the content elements provided to the at least one user based on the difference between the user state of the at least one user and the exit user state;

In accordance with a further aspect, the method may include modifying auxiliary stimulus provided to the at least one user.

In accordance with a further aspect, the modifying one or more of the content elements may include transitioning between one or more content samples.

In accordance with a further aspect, the modifying one or more of the content elements may include pausing one or more of the content elements.

In accordance with a further aspect, the modifying one or more of the content elements includes pausing one or more of the content elements at time codes associated with natural breaks in the one or more content elements.

In accordance with a further aspect, the method further includes adjusting the interval based on natural breaks in the one or more of the content elements.

In accordance with a further aspect, the content may include at least a first and a second time-coded content sample, and the modifying one or more of the content elements may include transitioning between a first defined time-code of the first time-coded content sample to a second defined time-code of the second time-coded content sample.

In accordance with a further aspect, the first defined time code is based on natural pauses in the first time-coded content sample and the second defined time code is based on natural pauses in the second time-coded content sample.

In accordance with a further aspect, the second time-coded content sample is selected from a plurality of time-coded content samples based on at least on of the first time-coded content sample.

In accordance with a further aspect, the selection of the second time-coded content sample is based in part on a prediction model.

In accordance with a further aspect, the content may include time-coded content, and the modifying one or more of the content elements may be based in part on a current time code in the time-coded content.

In accordance with a further aspect, the user state includes a brain state.

In accordance with a further aspect, the content elements have modifications applied at a specific change profile.

In accordance with a further aspect, the trigger user state comprises reaching a time code in the content.

In accordance with an aspect, there is provided a process or a use of time-coded content to induce a change is state of at least one user by presenting the time-coded content to the at least one user and using a bio-signal sensor. The time-coded content can include one or more content elements, one or more content modification processes. The content modification processes can include a modification, a trigger, a target user state, and at least one interval. The content modification processes can be configured to initiate the modification on detecting that the trigger is satisfied, modify one or more of the content elements based in part on the modification during the at least one interval, and modify one or more of the content elements based on a difference between a user state of the at least one user after the at least one interval, the target user state, and the modification.

In accordance with a further aspect, the trigger can include a trigger user state that the at least one user must satisfy and the modify one or more of the content elements based in part on the modification comprises modifying the one or more content element based in part on the user state.

In accordance with a further aspect, the trigger may include a time code in the content, and the modify one or more of the content elements based in part on the modification comprises modifying one or more of the content elements at or after the time code.

In accordance with a further aspect, the bio-signals of the at least one user may include bio-signals of a plurality of users, and the user state may be based on each user of the plurality of users.

In accordance with a further aspect, the user state may be determined based in part on a prediction model.

In accordance with a further aspect, the system further comprising a server configured to store the prediction model and provide the prediction model to the at least one computing device. The at least one computing device is configured to update the prediction model based on the difference between the user state of the at least one user after the at least one interval and the target user state.

In accordance with a further aspect, the prediction model comprises a neural network.

In accordance with a further aspect, the prediction model may be based in part on a user profile.

In accordance with a further aspect, the prediction model may be based in part on data from one or more other users.

In accordance with a further aspect, the one or more other users may share a characteristic with the at least one user.

In accordance with a further aspect, the at least one interval may be based in part on a current user state of the at least one user.

In accordance with a further aspect, the at least one interval is based in part on the content.

In accordance with a further aspect, the at least one interval is based in part on user input.

In accordance with a further aspect, the target user state is based in part on the content.

In accordance with a further aspect, the target user state may be based in part on input.

In accordance with a further aspect, the trigger user state is based in part on the content.

In accordance with a further aspect, the trigger user state may be based in part on input.

In accordance with a further aspect, modifying the one or more of the content elements is based in part on user input.

In accordance with a further aspect, at least one content modification process can be configured to determine a first user state of the at least one user using the bio-signals of the at least one user, apply a probe modification to one or more of the content elements provided to the at least one user, compute a difference between the first user state of the at least one user and the user state of the at least one user after a probe interval using the bio-signals of the at least one user, update at least one of the modification, the target user state, the trigger, and the at least one interval of one or more content modification processes based on a difference between the first user state and the user state of the at least one user after the probe interval.

In accordance with a further aspect, at least one content modification process is configured to determine a first user state of the at least one user using the bio-signals of the at least one user before a probe interval, compute a difference between the first user state of the at least one user before the probe interval and a user state of the at least one user after the probe interval using the bio-signals of the at least one user, update at least one of the target user state and the trigger user state based on the difference between the first user state and the user state after the probe interval.

In accordance with a further aspect, the content modification process can further comprise an exit user state and can be further configured to modify one or more of the content elements provided to the at least one user based on the difference between the user state of the at least one user during the at least one interval and the exit user state,

In accordance with a further aspect, the at least one bio-signal sensor may include at least one of EEG, EOG, EKG, EMG, PPG, heart rate, breath, sweat, gyroscopic, accelerometer, magnetometer, IMU, movement, vibration, sound, pulse wave amplitude, fNIRS, temperature, pressure, and electrodermal conductance sensors.

In accordance with a further aspect, the at least one user effector may include at least one of earphones, speakers, a display, a scent diffuser, a heater, a climate controller, a drug infuser or administrator, an electric stimulator, a medical device, a system to effect physical or chemical changes in the body, restraints, a mechanical device, a vibrotactile device, and a light.

In accordance with a further aspect, the content modification process may be further configured to modify auxiliary stimulus provided to the at least one user.

In accordance with a further aspect, the modify one or more of the content elements may include transitioning between one or more content samples.

In accordance with a further aspect, the modify one or more of the content elements may include pausing one or more of the content elements.

In accordance with a further aspect, the modify one or more of the content elements includes pausing one or more of the content elements at time codes associated with natural breaks in the one or more content elements.

In accordance with a further aspect, the content modification process adjusts the interval based on natural breaks in the one or more of the content elements.

In accordance with a further aspect, the time-coded content may include at least a first and a second time-coded content sample, and the modify one or more of the content elements may include transitioning between a first defined time-code of the first time-coded content sample to a second defined time-code of the second time-coded content sample.

In accordance with a further aspect, the first defined time code is based on natural pauses in the first time-coded content sample and the second defined time code is based on natural pauses in the second time-coded content sample.

In accordance with a further aspect, the second time-coded content sample is selected from a plurality of time-coded content samples based on at least on of the first time-coded content sample.

In accordance with a further aspect, the selection of the second time-coded content sample is based in part on a prediction model.

In accordance with a further aspect, the user state can include a brain state.

In accordance with a further aspect, the content elements can have modifications applied at a specific change profile.

In accordance with a further aspect, the trigger user state can include reaching a time code in the content.

In accordance with an aspect, there is provided a computer system to develop time-coded content for achieving an ultimate state by modifying content elements provided to at least one user, the system includes at least one computing device in communication with at least one bio-signal sensor and at least one user effector, the at least one bio-signal sensor configured to measure bio-signals of at least one user, the at least one user effector configured to provide time-coded content to the at least one user, wherein the time-coded content includes one or more content elements. The at least one computing device can be configured to provide the time-coded content to the at least one user via the at least one user effector, determine an initial user state of the user at a time code, modify one or more of the content elements provided to the at least one user, determine a final user state of the user after a test interval, update the time-coded content to provide a content modification process including a target user state, an interval, a modification, and at least one of a time code and a trigger user state, wherein the trigger user state is based on the initial user state, the target user state is based on the final user state, the interval is based on the test interval, and the modification and the time code are based on the modify one or more of the content elements.

In accordance with a further aspect, the at least one computing device can be further configured to determine another initial user state of the at least one user at another time code, wherein the another initial user state is determined with or after the final user state, modify one or more of the content elements provided to the at least one user, determine another final user state of the at least one user after another test interval, update the time-coded content to provide at least one more content modification process including a target user state, an interval, a modification, and at least one of a time code and a trigger user state, wherein the trigger user state is based on the another initial user state, the target user state is based on the another final user state, the interval is based on the another test interval, and the modification and the time code are based on the modify one or more of the content elements.

In accordance with a further aspect, the time code can include at least one of a regular, a random, a pre-defined, an algorithmically defined, a user defined, and a triggered time code.

In accordance with a further aspect, the interval may include at least one of a regular, a random, a pre-defined, a user defined, and an algorithmically defined interval.

In accordance with a further aspect, the modification may include at least one of a random, a pre-defined, a user defined, and an algorithmically defined modification.

In accordance with a further aspect, the time-coded content can be pre-processed to extract one or more content elements.

In accordance with a further aspect, the at least one user effector can be configured to provide time-coded content to a plurality of users and the user state can be based on the bio-signals of each user of the plurality of users.

In accordance with a further aspect, the content modification processes can be based in part on a user profile.

In accordance with a further aspect, the interval can be based in part on a current user state of the at least one user.

In accordance with a further aspect, the content modification processes can further comprise an exit user state based on the final user state, the ultimate user state, and the modify one or more of the content elements.

In accordance with a further aspect, the at least one bio-signal sensor can include at least one of EEG, EOG, EKG, EMG, PPG, heart rate, breath, sweat, gyroscopic, accelerometer, magnetometer, IMU, movement, vibration, sound, pulse wave amplitude, fNIRS, temperature, pressure, and electrodermal conductance sensors.

In accordance with a further aspect, the at least one user effector can include at least one of earphones, speakers, a display, a scent diffuser, a heater, a climate controller, a drug infuser or administrator, an electric stimulator, a medical device, a system to effect physical or chemical changes in the body, restraints, a mechanical device, a vibrotactile device, and a light.

In accordance with a further aspect, the system can further include one or more auxiliary effectors configured to provide stimulus to the at least one user and the computing device can be further configured to modify the stimulus provided to the at least one user by the auxiliary effector.

In accordance with a further aspect, the modify one or more of the content elements can include transitioning between one or more content samples.

In accordance with a further aspect, the modify one or more of the content elements can include pausing one or more of the content elements.

In accordance with a further aspect, the modify one or more of the content elements includes pausing one or more of the content elements at time codes associated with natural breaks in the one or more content elements.

In accordance with a further aspect, the computing device is further configured to adjust the interval based on natural breaks in the one or more of the content elements.

In accordance with a further aspect, the time-coded content can include at least a first and a second time-coded content sample and the modify one or more of the content elements can include transitioning between a first defined time code of the first time-coded content sample to a second defined time code of the second time-coded content sample.

In accordance with a further aspect, the first defined time code is based on natural pauses in the first time-coded content sample and the second defined time code is based on natural pauses in the second time-coded content sample.

In accordance with a further aspect, the second time-coded content sample is selected from a plurality of time-coded content samples based on at least on of the first time-coded content sample.

In accordance with a further aspect, the user state can comprise a brain state.

In accordance with a further aspect, the content elements have modifications applied at a specific change profile.

In accordance with an aspect, there is provided a method to develop time-coded content for achieving an ultimate user state by modifying content elements provided to at least one user. The method includes providing the time-coded content to the at least one user, the time-coded content including one or more content elements, determining an initial user state of the at least one user at a time code using bio-signals of the at least one user, modifying one or more of the content elements provided to the at least one user, determining a final user state of the user after a test interval, updating the time-coded content to provide a content modification process including a target user state, an interval, a modification, and at least one of a time code and a trigger user state, wherein the trigger user state is based on the initial user state, the target user state is based on the final user state, the interval is based on the test interval, and the modification and the time code are based on the modifying one or more of the content elements.

In accordance with a further aspect, the method can further include determining another initial user state of the at least one user at another time code, wherein the another initial user state is determined with or after the final user state, modifying one or more of the content elements provided to the at least one user, determining another final user state of the at least one user after another test interval, and updating the time-coded content to provide at least one more content modification process including a target user state, an interval, a modification, and a time code and a trigger user state, wherein the trigger user state is based on the another initial user state, the target user state is based on the another final user state, the interval is based on the another test interval, and the modification and the time code are based on the modifying one or more of the content elements.

In accordance with a further aspect, the time code can include at least one of a regular, a random, a pre-defined, an algorithmically defined, a user defined, and a triggered time code.

In accordance with a further aspect, the interval can include at least one of a regular, a random, a pre-defined, a user defined, and an algorithmically defined interval.

In accordance with a further aspect, the modification can include at least one of a random, a pre-defined, a user defined, and an algorithmically defined modification.

In accordance with a further aspect, the time-coded content can be pre-processed to extract one or more content elements.

In accordance with a further aspect, the at least one user can include a plurality of users, the user state can be based on the bio-signals of each user of the plurality of users.

In accordance with a further aspect, the content modification processes can be based in part on a user profile.

In accordance with a further aspect, the interval can be based in part on a current user state of the at least one user.

In accordance with a further aspect, the content modification processes can further comprise an exit user state based on the final user state, the ultimate user state, and the modify one or more of the content elements.

In accordance with a further aspect, the method can further include modifying auxiliary stimulus provided to the at least one user.

In accordance with a further aspect, the modifying one or more of the content elements can include transitioning between one or more content samples.

In accordance with a further aspect, the modifying one or more of the content elements can include pausing one or more of the content elements.

In accordance with a further aspect, the modify one or more of the content elements comprises pausing one or more of the content elements at time codes associated with natural breaks in the one or more content elements.

In accordance with a further aspect, the computing device is further configured to adjust the interval based on natural breaks in the one or more of the content elements.

In accordance with a further aspect, the time-coded content can include at least a first and a second time-coded content sample and the modifying one or more of the content elements can include transitioning between a first defined time code of the first time-coded content sample to a second defined time code of the second time-coded content sample.

In accordance with a further aspect, the first defined time code is based on natural pauses in the first time-coded content sample and the second defined time code is based on natural pauses in the second time-coded content sample.

In accordance with a further aspect, the second time-coded content sample is selected from a plurality of time-coded content samples based on at least on of the first time-coded content sample.

In accordance with a further aspect, the user state can include a brain state.

In accordance with a further aspect, the content elements can have modifications applied at a specific change profile.

In accordance with an aspect, there is provided a computer system to detect a user state of at least one user. The system including at least one computing device in communication with at least one bio-signal sensor, and at least one other signal sensor. The at least one bio-signal sensor configured to measure bio-signals of at least one user. The at least one other signal sensor configured to measure other signals of the at least one user. The at least one computing device configured to measure the bio-signals of the at least one user, measure the other signals of the at least one user, determine a user state of the at least one user using the measured bio-signals and a prediction model, update the prediction model with the determined user state and the measured other signals of the at least one user, determine the user state of the at least one user using the measured other signals and the updated prediction model.

In accordance with a further aspect, the system may be further configured to perform an action based on the user state determined using the measured other signals and the updated prediction model.

In accordance with a further aspect, the system further comprising a server configured to store the prediction model and provide the prediction model to the at least one computing device. The at least one computing device is configured to update the prediction model on the server.

In accordance with a further aspect, the prediction model comprises a neural network.

In accordance with a further aspect, the other signals may include at least one of a typing speed, a temperature preference, ambient noise, a user objective, a location, ambient temperature, an activity type, a social context, a user preferences, self-reported user data, dietary information, exercise level, activities, dream journals, emotional reactivity, behavioural data, content consumed, contextual signals, search history, and social media activity.

In accordance with a further aspect, the other signals may include bio-signals or behaviours of other individuals.

In accordance with a further aspect, the prediction model may be based in part on a user profile.

In accordance with a further aspect, the prediction model may be based in part on data from one or more other users.

In accordance with a further aspect, the one or more other users may share a characteristic with the at least one user.

In accordance with a further aspect, the at least one bio-signal sensor may comprise at least one of EEG, EOG, EKG, EMG, PPG, heart rate, breath, sweat, gyroscopic, accelerometer, magnetometer, IMU, movement, vibration, sound, pulse wave amplitude, fNIRS, temperature, pressure, and electrodermal conductance sensors.

In accordance with a further aspect, the user state can include a brain state.

In accordance with an aspect, there is provided a method to detect a user state of at least one user. The method including measuring bio-signals of at least one user, measuring other signals of the at least one user, determining a user state of the at least one user using the measured bio-signals and a prediction model, updating the prediction model with the determined user state and the measured other signals of the at least one user, determining the user state of the at least one user using the measured other signals and the updated prediction model.

In accordance with a further aspect, the method may further include performing an action based on the user state determined using the measured other signals and the updated prediction model.

In accordance with a further aspect, the prediction model comprises a neural network.

In accordance with a further aspect, the other signals may include at least one of a typing speed, a temperature preference, ambient noise, a user objective, a location, ambient temperature, an activity type, a social context, a user preferences, self-reported user data, dietary information, exercise level, activities, dream journals, emotional reactivity, behavioural data, content consumed, contextual signals, search history, and social media activity.

In accordance with a further aspect, the other signals may include bio-signals or behaviours of other individuals.

In accordance with a further aspect, the prediction model may be based in part on a user profile.

In accordance with a further aspect, the prediction model may be based in part on data from one or more other users.

In accordance with a further aspect, the one or more other users share a characteristic with the at least one user.

In accordance with a further aspect, the user state can include a brain state.

In accordance with an aspect, there is provided a computer system to map user states. The system including at least one computing device in communication with at least one bio-signal sensor and at least one user effector. The at least one bio-signal sensor configured to measure bio-signals of at least one user. The at least one user effector configured to provide stimulus to the at least one user. The at least one computing device configured to determine an initial user state, provide stimulus to the at least one user, determine a final user state, update a user state map using the stimulus, initial user state, final user state.

In accordance with a further aspect, the user state map can be updated using a time code at which the stimulus was provided to the at least one user.

In accordance with a further aspect, the computing device may be further configured to receive user input on the initial user state or the final user state that describes the desirability of the state.

In accordance with a further aspect, the computing device may be further configured to provide stimulus to the at least one user that is predicted to direct the at least one user into desirable user states.

In accordance with a further aspect, the determine the final user state may include determining the final user state after an interval.

In accordance with a further aspect, the stimulus may include modification of content presented to the at least one user, and the update a user state map may include generating content modification process that includes a trigger user state based on the initial user state, a target user state based on the final user state, and a modification based on the modification of content presented to the at least one user.

In accordance with a further aspect, the computing device may be further configured to induce the target user state by initiating the content modification process when the at least one user achieves the trigger user state.

In accordance with a further aspect, the user state map may be associated with a user profile of the at least one user and the system may be further be configured to apply the content modification process to other content when the user achieves the trigger user state.

In accordance with an aspect, there is provided a method to map user states, the method including determining an initial user state, providing stimulus to the at least one user, determining a final user state, updating a user state map using the stimulus, initial user state, final user state.

In accordance with a further aspect, updating the user state map includes updating the user state map using a time code at which the stimulus was provided to the at least one user.

In accordance with a further aspect, the method may further include receiving user input on the initial user state or the final user state that describes the desirability of the state.

In accordance with a further aspect, the method may further include providing stimulus to the at least one user predicted to direct the at least one user into desirable user states.

In accordance with a further aspect, the determining the final user state may include determining the final user state after an interval.

In accordance with a further aspect, the stimulus may include modification of content presented to the at least one user, and the updating a user state map may include generating content modification process that may include a trigger user state based on the initial user state, a target user state based on the final user state, and a modification based on the modification of content presented to the at least one user.

In accordance with a further aspect, the method may further include inducing the target user state by initiating the content modification process when the at least one user achieves the trigger user state.

In accordance with a further aspect, the method may further comprise associating the user state map with a user profile of the at least one user, and applying the content modification process to other content when the user achieves the trigger user state.

In accordance with an aspect there is provided a non-transient computer readable medium containing program instructions for causing a computer to perform any of the methods described herein.

In accordance with an aspect there is provided a hardware processor configured to assist in achieving a target brain state by processing bio-signals of at least one user captured by at least one bio-signal sensor and triggering at least one user effector to modify one or more of content elements. The hardware processor executing code stored in non-transitory memory to implement operations described in the description or drawings.

In accordance with an aspect there is provided a method to assist in achieving a target brain state by processing, using a hardware processor, bio-signals of at least one user captured by at least one bio-signal sensor and triggering at least one user effector to modify one or more of content elements, the method including steps described in the description or drawings.

### DESCRIPTION OF THE FIGURES

In the figures,

FIG. 1A illustrates a block schematic diagram of an example system, according to some embodiments.

FIG. 1B illustrates a block schematic diagram of an example system making use of user state triggered content modification processes, according to some embodiments.

FIG. 1C illustrates a block schematic diagram of an example system making use of periodic state determination, according to some embodiments.

FIG. 1D illustrates a block schematic diagram of an example system making use of content triggered modifications, according to some embodiments.

FIG. 2A illustrates an example content modification process wherein the user achieved the target user state, according to some embodiments.

FIG. 2B illustrates an example content modification process wherein the user did not achieve the target user state and the content is modified to reverse the first modification, according to some embodiments.

FIG. 2C illustrates another example content modification process wherein the user did not achieve the target user state and the content is modified to partly reverse the first modification, according to some embodiments.

FIG. 2D illustrates an example content modification process wherein final level of content modification is based on the user state, according to some embodiments.

FIG. 3 illustrates an example content modification process involving a pause, according to some embodiments.

FIG. 4 illustrates an example content modification processes involving the modification of one content element, according to some embodiments.

FIG. 5 illustrates an example time-coded content modification, according to some embodiments.

FIG. 6 illustrates example content made from content samples, according to some embodiments.

FIG. 7 illustrates example time-coded content with defined content modification process points, according to some embodiments.

FIG. 8 illustrates the content modification process, according to some embodiments.

FIG. 9 illustrates a block schematic diagram of an example system that can update content, according to some embodiments.

FIG. 10 illustrates the an example content development process, according to some embodiments.

FIG. 11 illustrates a block schematic diagram of an example system that can map user states, according to some embodiments.

FIG. 12 illustrates the an example user state mapping process, according to some embodiments.

FIG. 13 illustrates a block schematic diagram of an example system that can associate other signals with user states, according to some embodiments.

FIG. 14 illustrates the an example other signal and brain state association process, according to some embodiments.

FIG. 15 is a schematic diagram of an example computing device suitable for implementing the systems in FIG. 1A, FIG. 1B, FIG. 1C, FIG. 1D, FIG. 9, FIG. 11, or FIG. 13, in accordance with an embodiment.

### DETAILED DESCRIPTION

When an individual is trying to go to sleep, they may need to bring their mind from an active and alert state, to a relaxed state, and finally into a sleep state. In an effort to relax, some individuals may use white noise machines, audio programs, or music at a low volume. This sort of stimulus can provide individuals with sufficient engagement to occupy a busy mind and bring it to a relaxed state. This level of engagement may be helpful to relax, but it may become detrimental to bringing a user into a sleep state. The volume may be too loud or the content may be too engaging. Similar problems may arise when attempting to bring about other user state changes.

A system may turn off using a timer, but that offers no guarantee that the individual will be asleep when the system shuts down. A system may remove a stimulus when the user is asleep but this may rouse the user and interfere with their sleep.

There exists a need for systems that use the internal user states (e.g., brain states) to assist a user in achieving a state change, or at least alternatives. There exists a need for systems that may adapt and change the presentation of content to permit users to engage with or disengage with the content as needed to change states (e.g., fall asleep). There exists a need for systems with improved and enhanced efficacy in a sleeping aide.

Some aspects of the present disclosure are directed at computer systems that use bio-signals from a user to determine their internal states and modify content to induce state changes. Some embodiments of these systems can also modulate the stimulus provided to a user at the point of transition from awake to asleep to trigger the individual to fall into a sleep state. Some embodiments of these systems can detect when the user is susceptible to entering a sleep state and can initiate a content modification process to add, remove, or alter stimulus provided to a user to bring them into a sleep state.

Systems, methods, and devices described herein provide an improved or alternative mode of guiding a user to an ultimate user state (e.g., a sleep state). In some embodiments, the systems, methods, and embodiments can detect a user's state and modify content to bring the user to the ultimate user state. For example, some systems can detect when a user is on the edge of sleep and cut the content to bring the user into a sleep state. These systems are principally directed at inducing sleep states, however the systems, methods and devices described herein may be effective at inducing other states as well (e.g., flow states, wakefulness states, fear states, alert states, altered states, etc.).

Drifting off to sleep can be thought of as landing an airplane. In the high energy of the day, the airplane flies high in the sky with many turbulent moments. When the day ends, users may need to shift into sleep and bring their energy level down and fade their awareness out until the plane lands in the safety of sleep. Methods described herein can, in some embodiments, assist a user in, for example, falling asleep by responding to the user's brain rhythms, helping the user disengage from the things that keep them awake.

Ideally, a user would be able to gradually fade their awareness out until unconsciousness in a smooth transition. In reality, the process of falling asleep can be modulate turbulently between unconscious, semi-conscious, and awake states. Methods described herein can use content (e.g., stories or soundscapes) combined with algorithms that work with these ups and downs and intelligently modifies the content to bring the user to rest.

The algorithm can, for example, determine when a user's consciousness is flickering and change the tone and/or pacing of the story.

In some embodiments, the methods described herein can detect when a user is nearing a sleep state and gracefully fade the content out at the right moment to assist a user in falling asleep. In some embodiments, the content can fade out during a moment of semi-consciousness which can cue a user to fall asleep. The user may still be partly conscious and aware that the content has faded out. In some embodiments, if the content fades out, but the user comes to an awake state, then the content can return and await another moment to fade out. In some embodiments, the fade out can test the user to determine how close to sleep they are.

Some systems, method, and devices described herein can provide dynamic content to the user intended to responsively direct the user to a variety of target user states beyond just sleep states such as alert states (studying and driving), wakefulness states (waking up), terror states (entertainment), altered states (therapy), etc.

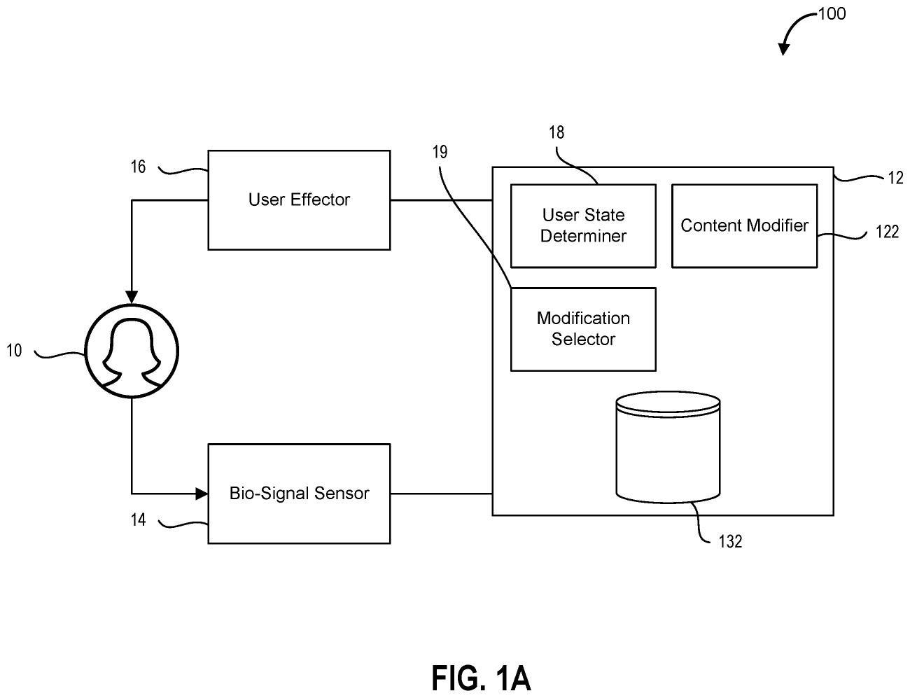

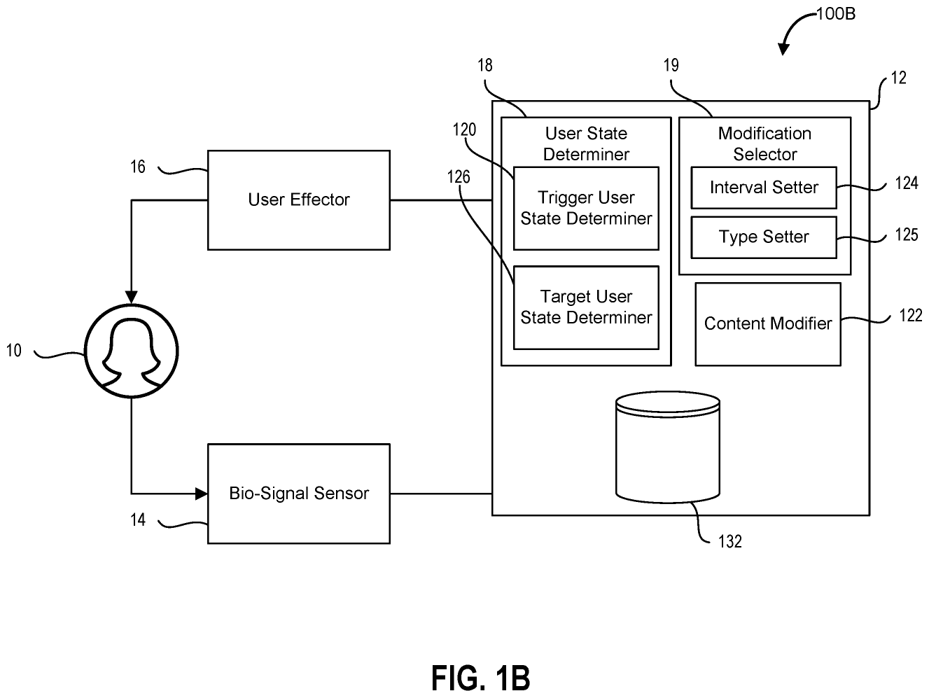

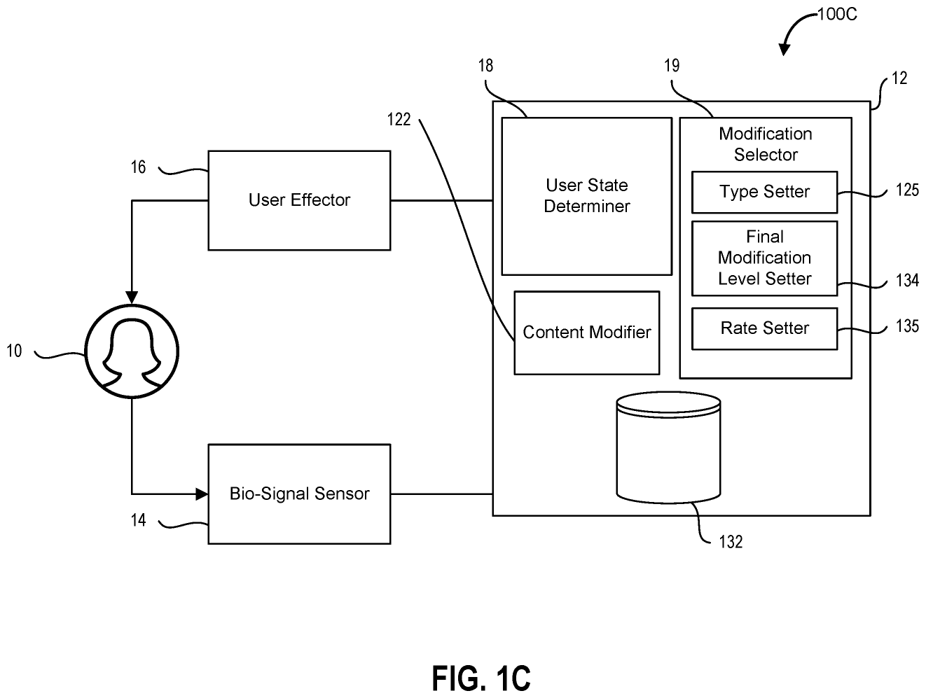

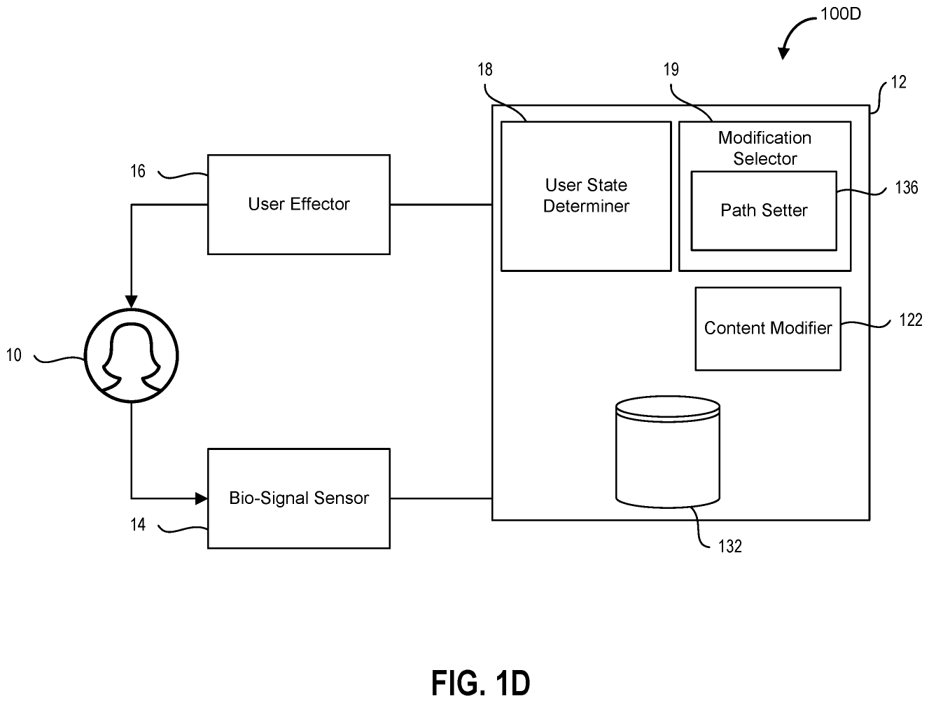

FIG. 1A illustrates a block schematic diagram of an example system, according to some embodiments.

The system 100 includes a bio-signal sensor 14, computing device 12, and user effector 16. Bio-signal sensor 14 is capable of receiving bio-signals from user 10. User effector 16 can provide content to user 10. Computing device 12 can be in communication with bio-signal sensor 14 and user effector 16. In operation, computing device 12 can provide content to user 10 via user effector 16. Bio-signal sensor 14 can receive bio-signals from user 10 and provide them to computing device 12. Computing device 12 can use the bio-signals to determine the user state of user 10 and initiate a content modification process with content provided to user 10. After an interval has elapsed, then computing device 12 can determine the difference between the user state of the user and the target user state and initiate further content modification based on the difference.

Computing device 12 may include a user state determiner 18, a modification selector 19, a content modifier 122, and a electronic datastore 132.

User state determiner 18 determines the state of user 10. In some embodiments the user state may be a brain state of user 10. User state determiner 18 may make use of bio-signals received from the bio-signal sensor 14 to determine the user state. User state determiner 18 may determine the user state based in part on one or more types of bio-signals (e.g., EEG signals, heart rate, skin conductance, etc.). User state determiner may make use of non-bio-signals to assist it in determining the user state. User state determiner 18 may make use of algorithms to determine the user state. In some embodiments, these algorithms can be based in part on a user profile. In some embodiments, these algorithms can be generated by or comprise machine learning techniques. User state determiner 18 can determine the user state on a continuous and/or periodic basis, or at defined times.

Modification selector 19 can determine a content modification process based on at least one of the user's state, the content, and a target or desired user state (e.g., a brain state). In some embodiments, modification selector 19 can be configured to generate content modification processes to modify content elements in a manner that has a higher predicted probability of driving the user to a target user state than not modifying the content elements. In some embodiments, the content modification process may be based on a probability that the user is in certain user state.

In some embodiments, content modification processes can involve a specific type of content modification, a trigger user state for the content modification, a target user state for the modification, and optionally a fail condition (e.g., failure to reach the target user state after a pre-defined interval). In some embodiments, content modification processes can be configured to provide a pre-defined rate of content modification (i.e., rate at which modification is applied to the content). In some embodiments, the content modification process can include a rate of content modification application, a final level of content modification, and an interval, wherein the final level of content modification can be based in part on the user state. In some embodiments, content modification processes can involve selecting a path that the user takes through the content based on the user state. Modification selector 19 can be configured to track prior content modifications to provide content modification processes that can maintain coherence of content (e.g., narrative coherence of a story).

Modification selector 19 can be configured to generate a set of content modification processes predicted to drive a user to a final target user state. For example, modification selector may generate a series of target user states (e.g., engagement, exhaustion, and diminished consciousness) to drive the user to a final target user state (e.g., sleep). For example, it may be effective if modification selector 19 is configured to engage the user with the content (i.e., an engagement state) prior to attempting to drive other user state changes in the user (e.g., driving them to sleep). In some embodiments, the modification selector 19 may monitor to apply several content modification processes in parallel (e.g., monitoring for two different trigger user states).

Content modifier 122 can modify a content element delivered to user 10. Content modifier 122 can increase or decrease features of the content (e.g., volume, audio fidelity, intensity, etc.), insert pauses in content elements of tracks, or transition between content samples. Content modifier 122 can make modifications to the content instantly or over a period of time. Modification selector 19 can control content modifier 122 directly or indirectly. Content modifier 122 can be configured to modify content generally, separate and apart from content modifications determined by modification selector 19 (e.g., it can be configured to filter high pitched noises from the content).

Electronic datastore 132 is configured to store various data utilized by system 100 including, for example, data reflective of user state determiner 18, modification selector 19, and content modifier 122. Electronic datastore 132 may also store training data, model parameters, hyperparameters, and the like. Electronic datastore 132 may implement a conventional relational or object-oriented database, such as Microsoft SQL Server, Oracle, DB2, Sybase, Pervasive, MongoDB, NoSQL, or the like.

Content can be stored in electronic datastore 132 or input into computing device 12 in another manner. In some embodiments, content can be stored elsewhere (e.g., in another server or datastore) and uploaded into computing device 12 for modification. In some embodiments, content can be continuously fed into computing device 12 (e.g., streamed into computing device 12 for modification). In some embodiments, content can be generated and/or uploaded into computing device 12 (e.g., content can be generated from a live-feed and modified in real time or near-real time using computing device 12). Other content storage and retrieval methods are also conceived.

In some embodiments, content modification processes include a trigger user state, a target user state, an interval, and a content modification type. In such processes, content modification is triggered when user state determiner 18 determines that the user has achieved the trigger user state (i.e., user state triggered). The content modification can be applied immediately in full or introduced over time into the content. For example, if the content modification is a volume decrease, the volume may be decreased to the lower volume immediately when the user achieves the trigger user state or the volume reduction may be initiated when the user achieves the trigger user state and decreases to the lower volume over a pre-defined time and/or at a pre-defined rate. The content modification process maintains the content modification until the interval has elapsed and then the user's state is again sampled to see if the user has achieved the target user state. The process can be configured to further modify the content based on the success or failure of the user to achieve the target user state after the interval. For example, referring back to volume reduction, the content modification process can be configured to maintain the reduced volume on successful achievement of the target user state or to completely silence the audio. Additionally, the content modification process can be configured to return to the original volume if the user has not met the target user state or the volume level can be determined based on the user's state after the interval (e.g., if the user has not met the target user state, then the degree to which volume is again increased is based on the difference between the user state and the target user state).

In some embodiments, the system can be configured to modify the content if the user achieves a user state for a predefined amount of time. In such embodiments, this can ensure that the trigger user state has a degree of permanence before initiating a modification based on that trigger user state.

In some embodiments, the content modification process includes a final level of content modification based on the user state, a rate of content modification application, and (optionally) an interval. For example, in some embodiments, the system may be configured to periodically sample the user state and determine a final level of content modification based on the periodically sampled user state. The content modification may apply at a fixed rate (or otherwise pre-determined rate) until the content modification level reaches the final content modification level. After the periodic interval, the system may sample the user state once more an repeat the process.

In some embodiments, the content has pre-defined time codes within it at which it will query the user state and apply a content modification based thereon. For example, the content might include decision points wherein the content determines which path through the narrative to take based on the user state. In other examples, the content may be configured to pause at specific times to avoid disrupting the flow of content delivery.

In some embodiments, the system may also be capable of triggering a modification where the user state has been stable for extended periods of time to determine whether the user is susceptible to a state change at that moment. This can be done if the user is not in a desired or trigger user state (e.g., a pre-sleep state), but has been in another state (e.g., a low energy state) for a long period of time. In some embodiments, the system can further be configured to apply content modification processes to content to ascertain the user's susceptibility to those processes. For example, as described above, the system can be configured to modify content to determine if the user is susceptible to a state change. In other embodiments, the system can be configured to apply different content modification processes to ascertain the susceptibility of the user to those content modification processes. For example, the system may decide to apply a cadence reducing modification to the pace of music to ascertain if such content modification processes can drive the user towards a desired user state.

Technical advantages of implementing content modification processes through a modification selector 19 include maintaining a level of coherence and/or consistency in the content. It can keep modifications in place for a pre-defined interval, change modification at a pre-defined rate, or select content modification processes so as not to conflict with each other. It can focus the user's attention on content itself rather than on the modifications. Put another way, it prevents the user's attention from being called to continuous modifications and/or to the content conflicting (e.g., constantly fluctuating volume) rather than the content.

In some embodiments, the modification selector 19 is configured to bring the user through a plurality of content modification processes. In some embodiments, the system may have several target user states for the user the achieve. For example, when falling asleep, it may be necessary to engage the user with the content before attempting to put the user to sleep. In such example systems, the early content modification processes can modulate the volume or action of a story to increase user engagement and once this state is successfully achieved, then attempt to put the user to, for example, sleep.

Some embodiments of system 100 can be implemented using a wearable device (e.g., headphones with onboard computing and bio-signal sensors). Some embodiments can separate the components of system 100 (e.g., wearable sensors provide bio-signals to a user's phone which in turn can instruct the user's television). Computing device 12 may also be combined with either of the user effector 16 or the bio-signal sensor 14.

In accordance with an aspect, there is provided a computer system for achieving a target user state by modifying content elements provided to at least one user 10. The system includes at least one computing device 12 in communication with at least one bio-signal sensor 14 and at least one user effector 16, the at least one bio-signal sensor 14 can be configured to measure bio-signals of at least one user 10, the at least one user effector 16 can be configured to provide content to the at least one user 10, wherein the content comprises one or more content elements. The at least one computing device 12 can be configured to provide the content to the at least one user 10 via the at least one user effector 16, compute a difference between the user state of the at least one user before an interval and the target user state using the bio-signals of the at least one user using user state determiner 18, modify one or more of the content elements provided to the at least one user during the interval based on the difference between the user state of the at least one user before the interval and the target user state using content modifier 122, compute a difference between the user state of the at least one user after the interval and the target user state using the bio-signals of the at least one user using user state determiner 18, modify one or more of the content elements provided to the at least one user after the interval based on the difference between the user state of the at least one user after the interval and the target user state using content modifier 122.

The following three embodiments illustrated in FIG. 1B, FIG. 1C, and FIG. 1D, show various embodiments of the system 100 intended to highlight specific possible functionality. These functions are not limited to the embodiments presented and can be combined with any or each of the other embodiments.

FIG. 1B illustrates a block schematic diagram of an example system making use of user state triggered content modification processes, according to some embodiments.

In some specific embodiments, the system is configured to sample the user to determine if the user has reached a trigger user state. In detecting a trigger user state, the system can be configured to select a type of content modification and an interval that this modification will be applied before resampling the user state. Once the interval has elapsed the system can resample the user state to determine whether they have achieved a target user state or not and possibly further modify the content based on that determination.

System 100B comprises some of the same components of system 100 and variations that apply to those of system 100 can equally be applied to the components of system 100B.

System 100B comprises a user state determine 18 that includes a trigger user state determiner 120 and a target user state determiner 126. System 100B further comprises a modification selector 19 that includes an interval setter 124 and a type setter 125.

Trigger user state determiner 120 may determine if user 10 has achieved a trigger user state. In some embodiments, the trigger user state may be a brain state of user 10. In some embodiments, the trigger user state may include the user achieving a particular state at a particular time code in the content. For example, the trigger user state may be that user 10 is in a pre-sleep state at the 8 s mark in the content.

Target user state determiner 126 can determine whether the user has achieved a target user state after the interval. Computing device 12 can, for example, determine that the user is distant from the target user state using target user state determiner 126 and modify the content with content modifier 122 to reverse the changes initiated when the trigger user state was achieved (e.g., if the content modification didn't successfully put user 10 to sleep, then the content can resume in its unmodified form to engage user 10). In another example, computing device 12 can determine that the user is at or near the target user state using target user state determiner 126 and not modify the content or modify the content with content modifier 122 to completely silence the content (e.g., the content can become quiet to induce sleep and if user 10 falls asleep because of this modification, the content can become completely silent).

Type setter 125 sets the type of content modification. Computing device 12 can be configured to modify a variety of content including audio, video, tactile, electrical, olfactory, physical, and other sensory content. Type setter 125 can determine which type of content is modified. For example, for audiovisual content, type setter 125 may decide to modify the audio, the visual, or both types of content. Type setter 125 can further be configured to determine the type of modification that will be carried out on the content. For example, audio content can have its volume altered, it can be filtered (e.g., removing vocal audio, but retaining melodic audio), or other modifications can be carried out. Visual content can be globally brightened or darkened, specific features in the content can be enhanced or diminished (e.g., blurring items in the visual content or enhancing them), or otherwise filtered or distorted. Type setter 125 can determine the type of content modification based in part on the content itself, algorithms, machine learning, modifications that have been successful for this user or others in the past, on an experimental basis, or through some other way.

Interval setter 124 sets the interval. Computing device 12 can modify content delivered to the user using content modifier 122 and may wait an interval to determine whether the user has achieved a target user state. Interval setter 124 can set intervals lasting pre-defined amount of time. Interval setter 124 can set the interval between content modification initiation and target user state determination. Interval setter 124 can set the interval based on the content (e.g., the content may include a predefined delay). Interval setter 124 can set the interval based on the modification (e.g., for volume decreases, the interval may be 5 s longer than the period over which content modifier 122 decreases the volume). Interval setter 124 can set the interval based on a current brain state of user 10 (e.g., if the system predicts that the user is highly susceptible to sleep, the interval setter 124 may set a relatively short interval to determine if sleep has taken user 10).

In accordance with an aspect there is provided a system 100 to assist at least one user 10 in achieving a target brain state. The system includes at least one computing device 12 in communication with at least one bio-signal sensor 14 and at least one user effector 16, the at least one bio-signal sensor 14 can be configured to measure bio-signals of at least one user 10, the at least one user effector 16 can be configured to provide content to the at least one user 10, wherein the content comprises one or more content elements. The at least one computing device 12 can be configured to provide the content to the at least one user via the at least one user effector 16, determine that a trigger user state has been achieved using the bio-signals of the at least one user using a trigger user state determiner 120, modify one or more of the content elements provided to the at least one user based on the achieved trigger user state using a content modifier 122, compute a difference between the brain state of the at least one user after an interval and the target user state using the bio-signals of the at least one user using target user state determiner 126, modify one or more of the content elements provided to the at least one user after the interval based on the difference between the brain state of the at least one user after an interval and the target brain state using content modifier 126.

Other embodiments that may trigger when the user enters a trigger user state may further be configured with a fail state instead of an interval. In these embodiments, the content modification is carried out when the user achieves the trigger user state, but reevaluates should the user enter a fail user state. Fail user state can, for example, represent changes in user state away from rather than towards the ultimate target user state. Some embodiments may be configured to implement both a fail user state and an interval. In such embodiments, the fail user state may provide a safeguard against content modification processes that have immediate adverse effects on the user state.

In accordance with a further aspect, computing a difference between the user state of the at least one user before an interval and the target user state using user state determiner 18 comprises determining that a trigger user state has been achieved using the bio-signals of the at least one user using trigger user state determiner 120.

FIG. 1C illustrates a block schematic diagram of an example system making use of periodic state determination, according to some embodiments.

The content modification processes can be configured to ensure a level of content coherence is maintained while the user's state changes. In some embodiments, the level of content modification may depend on the user state, but the rate at which the modification is incorporated into the content remains fixed (or otherwise pre-determined). For example, the volume level may be set to decrease by, for example, ten or twenty percentage points depending on the user state, but in both situations, the rate of volume reductions could be fixed at one percentage point every second until the final volume level is achieved (e.g., 10 s for a decrease of ten percentage points and 20 s for a decrease of twenty percentage points).

System 100C comprises some of the same components of systems 100 and 100B and variations that apply to those of systems 100 and 100B can equally be applied to the components of system 100C.

System 100C further comprises a modification selector 19 that includes a rate setter 135, final modification level setter 134, and a type setter 125.

As described above, the type setter 125 can set the type of modification that is to be carried out.

Final Modification Level Setter 134 sets the final level of modification that is to be applied to the content. In some embodiments, the final modification level can be based in part on the user state. In some embodiments, the final modification level can be based on the probability that a user is in one of one or more user states.

Rate setter 135 sets the rate at which the modification is carried out. The rate can be a linear rate, exponential, or some other rate profile. The system may be configured such that rate setter 135 is capable of fully applying the final modification level to the content prior to any subsequent user state determinations. If the user state meets a new trigger user state while the modification is being applied, then the rate may be changed (e.g., if the system is trying to put user 10 to sleep and sees that they are rapidly entering an alert state, it may halt any ongoing content modifications).

In some embodiments, the system may be configured to periodically sample the user state (or periodically act on continuously sampled user states). In such embodiments, the system may determine the user state with user state determiner 18. The modification selector 19 may then choose to modify the volume level using type setter 125, decide, using the user state, that the final volume level will be fifty percentage points lower than it currently is using the final modification level setter 134, and set the rate for this decrease to a rate of four percentage points a second using rate setter 135. In some example embodiments, the user state can be a probability that the user is in an awake state and the final volume set by final modification level setter 134 could be proportional to the probability that the user is awake (e.g., if the user is in a state that has a fifty percent probability of being an awake state, then the volume can be set to fifty percent of the raw volume level).

In accordance with a further aspect, the content elements may have modifications applied at a specific change profile using rate setter 135. These change profiles can include linear rates, geometric rates, exponential rates, or other mathematically determined rates. The change profiles may also be based on perceptual experience of the user in that the change profile is calibrated to increase or decrease at a rate that is perceived to be linear or some other fade in or fade out by the user. The change profile may also be user defined or selected.

FIG. 1D illustrates a block schematic diagram of an example system making use of content triggered modifications, according to some embodiments.

In some embodiments, the content is modified by modifying the path the user takes through the content. For example, if content is a narrative, then the content can be modified by selecting a path through the narrative to present to the user based on their user state at various decision points embedded within the content at time codes. For example, if a user is trying to have a story told to them to lull them to sleep, then the narrative can start in a high energy and engaging narrative and as the user grows weary, the story can gradually choose paths through the story that are lower energy to drive the user into a sleep state.

System 100D comprises some of the same components of systems 100, 100B, and 100C and variations that apply to those of systems 100, 100B, and 100C can equally be applied to the components of system 100D.

System 100D comprises a modification selector 19 that includes a path setter 136. The path setter 136 can act as a narrative engine. The path setter 136 can dynamically change the experience provided to the user. At points in the narrative, path setter 136 can select a path through the narrative based on the user state as determined by user state determiner 18.

In such embodiments, modification selector 19 can further be configured to track the narrative path the user has taken through the content and ensure coherence for future paths chosen by the path setter 136. For example, the content may be pre-configured with a branching path through the narrative. When path setter 136 sets a path at a decision point, modification selector 19 can remove future branches from the narrative that would not make narrative sense.

In some embodiments, certain paths can be taken at many decision points. Path setter 136 may set the path to move through this content. Modification selector 19 can then track that this path has been exhausted and ensure that it is not given to path setter 136 nor presented to the user 10 a second time.

In some embodiments, path setter 136 may input a pause at certain decision points. For example, if the user appears to be verging on sleep (trigger user state) at the end a sentence (a natural pause point), path setter 136 may insert a pause into the narrative that lasts a certain interval. If after the interval has elapsed, the user has fallen asleep (target user state) then the narration stops. If the user has not moved into a sleep state, then the narration may continue. In this way, system 100D may use aspects described in the context of system 100B.

In some embodiments, the narrative is generated procedurally or using machine learning in a dynamic manner while it is being presented to the user. In such embodiments, the modification selector 19 can adapt narrative elements of the content as path setter 136 works through the content. In some embodiments, the narrative can be procedurally generated from input from the narrative itself (to generate a perpetually generating narrative) or from input from user states (to generate engaging content).

For stories to act as potent transformative tools they need to make narrative sense. The narrative needs to guide the user. The narrative gives the user a model to relate to what is happening and ensure that whatever stimulus is given to the user fits with the narrative.

Modification selector 19 can carry out any, all, or some combinations described above in the context of systems 100B, 100C, and 100D. For example, modification selector 19 may implement a path setter 136 to move through narrative content in addition to trigger user state determiner 120 to trigger a volume reduction in the narration and a final modification level setter 134 to set the background music level.

In accordance with a further aspect, the at least one user effector 16 may be configured to provide content to a plurality of users 10, and the user state can be based on the bio-signals of each user of the plurality of users 10. In these embodiments, any trigger or target state may be a shared state between the plurality of user. For example, a couple trying to sleep may both listen to the same content. System 100 may detect bio-signals from both parties using two bio-signal sensors 14. Computing device 12 may trigger a content modification when both of the users 10 are at or near a sleep state in an attempt to induce a sleep state in the couple. In another example, computing device 12 may trigger a content modification process when one member of the users 10 is near a sleep state in order to induce sleep in that user 10, but computing device 12 may continue to provide content to the other users 10.

In accordance with a further aspect, the user state may be determined based in part on a prediction model. In some embodiments, the user state can be the state that the system predicts a user needs to have achieved in order to have a state change induced. For example, the user state can be a pre-sleep state that the system predicts the user will need to be in to fall asleep when the volume fades out.

In accordance with a further aspect, the system 100 may further include a server configured to store the prediction model and provide the prediction model to the at least one computing device 12. The at least one computing device 12 may be configured to update the prediction model based on the difference between the user state of the at least one user after the interval and the target user state. In some embodiments, the prediction model will update based on the success or failure of the system in inducing the target user state in the user. The difference between the user state after the interval and the target user state can be an indication of success or failure in inducing the target user state, a mathematical difference or distance measure between the states, or another mode of comparing the two states. In some embodiments this update may affect prediction models for other users. In some embodiments, these updates may be confined to apply only to the specific user in question.

In accordance with a further aspect, the prediction model comprises a neural network. In some embodiments, the neural network can be trained before system 100 is implemented, updated from time to time, or updated based on use in system 100.

In accordance with a further aspect, the prediction model may be based in part on a user profile. In some embodiments, the user profile can include characteristics that the user inputs themselves. In some embodiments, the user profile may include user preferences. In some embodiments, the user profile may include historic data from the user. In some embodiments, the system may use historic data from the user to provide a tailored content experience to the user (e.g., uses particular modulations at particular times that work for the user). In some embodiments, the user profile can include medical history and related data sets. In some embodiments, the user profile can include medical imaging, genetic data, metabolic data, clinical treatment records, etc. In some embodiments, the user profile may be provided by a third party (e.g., a physician or other professional). In some embodiments, the user profile may have a user state map associated with it to assist system 100 in determining when to initiate a content modulation to induce a state change in user 10.

In accordance with a further aspect, the prediction model may be based in part on data from one or more other users. In some embodiments, the system may aggregate data from a population. In these embodiments, the system may, for example, determine the time code in content where, if the volume cuts out, users are most likely to fall asleep. The system may also determine trigger user states most likely to induce sleep should content then be modulated. In some embodiments, the prediction model is based in part on population data to provide interventions based on the user's clinical information (e.g., subsets with similar medical conditions).

In accordance with a further aspect, the one or more other users may share a characteristic with the at least one user 10. For example, they may share biographical information or have similar medical conditions. In some embodiments, the system may tailor the content experience based on data aggregated from other users that are similar to the user 10.

In accordance with a further aspect, the prediction model may be based in part on user preferences. In some embodiments, the prediction model may be based in part on a model used for another specific user (e.g., a prototypical or otherwise idealized model, a model based on a celebrity).

In accordance with a further aspect, the interval may be based in part on a current user state of the at least one user 10. For example, if a user is determined to be in a state very likely to enter a sleep state, then the interval may be shorter to ascertain whether the user has successfully entered the sleep state. In an alternative example, the system may determine that the user is likely to enter a sleep state after a longer interval and define the interval accordingly.

In accordance with a further aspect, the interval may be based in part the content. In some embodiments, the content itself may have time codes at which it will assess the user's state to determine the user state. For example, a story may switch to a less action-packed version when it detects that the user is close to sleep, the system may then detect whether the user has entered a sleep state after a specific interval that allows the story to switch back to the more action-packed original version while maintaining coherence of the story.

In accordance with a further aspect, the interval is based in part on user input. For example, the user may prefer intervals of a certain duration. For example, the user may configure the system to use pauses of no more than 1.5 s in a story to see if the user is falling asleep.

In accordance with a further aspect, the target user state may be based in part on the content. In some embodiments, the content itself may have particular target user states defined at certain parts. For example, a story may have portions where it lulls a user 10 into safety in order to effectively scare them.

In accordance with a further aspect, the target user state may be based in part on input. In some embodiments, the user 10 may choose what ultimate user state they are trying to achieve. The system 100 may further define intermediate target user states to bring the user 10 to the ultimate user state. In some embodiments, the user may be able to provide a manual input (e.g., a subtle head nod) to trigger the content delivery to continue. In such embodiments, the user is provided with a manual override to system 100's default path and the target user state can be characterized as requiring the user to not provide such input.

In accordance with a further aspect, the trigger user state may be based in part on the content. In some embodiments, the content itself may have particular trigger user states defined at certain parts. For example, a story may have portions where it lulls a user 10 into safety in order to effectively scare them.

In accordance with a further aspect, the trigger user state may be based in part on input. The system may further define intermediate states to bring the user to the ultimate user state.

In accordance with a further aspect, the modify the one or more of the content elements is based in part on user input. For example, the user may have preferred types of content modification (e.g., content fade outs), that they configure the system to provide with modification selector 19.

In accordance with a further aspect, the at least one computing device 12 may be further configured to determine a first user state of the at least one user 10 using the bio-signals of the at least one user 10, apply a probe modification to one or more of the content elements provided to the at least one user using content modifier 122, compute a difference between the first user state of the at least one user and the user state of the at least one user after a probe interval set with interval setter 124 using the bio-signals of the at least one user, and update at least one of the target user state and the trigger user state based on the difference between the first user state and the brain state after the probe interval using modification selector 19. The system may be configured to probe the user to determine their susceptibility to a state change. In some embodiments, if the user is trying to sleep, the system may decrease the volume slightly and monitor the effect on the user's level of alertness and modify any subsequent trigger and target user states based on the user's level of alertness. For example, the system may determine that the user's alertness level decreased drastically in response to a slight volume decrease and may alter the trigger user states to more easily capture the user.

In accordance with a further aspect, the at least one computing device 12 is further configured to determine a first user state of the at least one user 10 using the bio-signals of the at least one user 10 before a probe interval, compute a difference between the first user state of the at least one user 10 before the probe interval and a user state of the at least one user after the probe interval using the bio-signals of the at least one user 10, and update at least one of the target user state and the trigger user state based on the difference between the first user state and the user state after the probe interval. The system may be configured to monitor the stability (or lack thereof) of the user state and update system variables in modification selector 19 based thereon.

In accordance with a further aspect, the computing device 12 may be further configured to compute a difference between the user state of the at least one user 10 during the interval and an exit user state using the bio-signals of the at least one user 10, and modify one or more of the content elements provided to the at least one user based on the difference between the user state of the at least one user 10 and the exit user state. In some embodiments, the modification selector 19 may monitor the user state during interval and cancel any content changes if it determines the user state is outside of acceptable thresholds. For example, if the user is attempting to sleep and has reached the trigger user state, then computing device 12 may decrease the volume with content modifier 122. If this volume decrease rouses the user into a state increases their alertness level (and consequently brings the user further away from the target user state), then the system may increase the volume to its original level using the content modifier 122 to prevent any further increase in alertness.

In accordance with a further aspect, the at least one bio-signal sensor 14 may include at least one of EEG, EOG, EKG, EMG, PPG, heart rate, breath, sweat, gyroscopic, accelerometer, magnetometer, IMU, movement, vibration, sound, pulse wave amplitude, fNIRS, temperature, pressure, and electrodermal conductance sensors. Other sensors that detect bio-signals of the user are also possible. System 100 may make use of different types of bio-signal sensors. Some embodiments may also use other signals to ascertain a brain state of the user.

In accordance with a further aspect, the at least one user effector 16 may include at least one of earphones, speakers, a display, a scent diffuser, a heater, a climate controller, a drug infuser or administrator, an electric stimulator, a medical device, a system to effect physical or chemical changes in the body, restraints, a mechanical device, a vibrotactile device, and a light. Other user effectors are also possible. The content may be provided by different types of user effectors at once (e.g., audiovisual content presented visually on a display and audibly through speakers).

In accordance with a further aspect, the system may further include one or more auxiliary effectors configured to provide stimulus to the at least one user, and the computing device may be further configured to modify the stimulus provided to the at least one user by the auxiliary effector. In some embodiments, computing device 12 may control auxiliary effectors. For example, content may be presented to a user to induce sleep on a tablet computer acting as the user effector 16 and computing device 12 may also control the lamp light level as an auxiliary effector. When computing device 12 determines that user 10 has achieved the ultimate sleep state, the computing device 12 may instruct the lamp to decrease the lighting level in response to the achieved sleep state.

### Exemplary Content and Modification Types

Content can include many things such as any one of soundscapes, music, stories (e.g., podcasts), videos, light shows, olfactory demonstrations, tactile experiences, exercise intensity (e.g., while working out to induce a flow state in the user), virtual reality content, electrical stimulation (e.g., electrical stimulation therapy), or other stimulus provided to the user or combinations thereof. Content can be pulled from external sources (e.g., the system can take raw content and apply modifications to induce state changes), or the content can be specifically configured to interoperate with system 100 (e.g., the content is embedded with particular content modification processes). Some embodiments may even pull raw content and process it to interoperate with system 100 (e.g., music may be pulled from an external source and processed to extract various tracks (vocals or melody) to individually modify).

Content elements can include, for example, the volume of the content, its playback speed, tracks, visual or audio content, brightness, level of vibration, aroma, degree of virtualization (e.g., in VR/AR environments, the degree to which objects are virtualized or animated or disassociated from present reality), degree of social connectivity (e.g., implementing “do not disturb” as a user comes closer to sleep), etc. The content modifier 122 can modify these content elements in a binary fashion (on or off), or in a gradient fashion (degree of the content element). The content modifier 122 can individually modify content elements of specific pieces of content (e.g., for content comprising a story being read with music provided in the background, contend modifier 122 can individually modify the cadence, pitch, path, or volume of the story without necessarily modifying those same elements in the background music). In some embodiments, the system can modify a plurality of content elements (e.g., volume of all audio tracks).

In some embodiments, content elements can also include separate content samples that content modifier 122 can switch between. For example, there may be content that comprises a story in which the user's state (or other metric or option) dictates the path that the user takes through the content. In some embodiments, the content modification will include transitioning between a primary track, to a transition track, and finally to a secondary track.

In some embodiments, content can also be procedurally or algorithmically generated. For example, content such as music (but not only music) can be broken down into more fundamental pieces such as which chords or notes play and at what volume. The content in such embodiments can be procedurally generated based on, for example, the user state wherein the user state dictates the probability that notes or chords will be played and at what volume. Example embodiments may dictate that only major or minor chords be played based on user state (e.g., if the user is sad, then only major chords, generally characteristic of upbeat music, be played, or if the user is too excited, then minor chords, generally associated with more somber music, be played). In some embodiments, the architecture of the content may be procedurally generated. For example, a bridge may be inserted based on, for example, the user state, to offer variety to the user when their attention wanes or to transition to a new section of the content. In some embodiments, the probability of moment-to-moment notes and chords played on one or more instruments can be based in part on user states. For example, alpha waves may be associates with the plano and the notes played using the plano are decided based in part on the user's current alpha wave outputs while other outputs control other instruments. This form of procedural generation may further incorporate other rules not based on user state (e.g., ensuring that the same notes or chords are not repeatedly played within a certain timeframe or otherwise entrenching content variety into the rules).

In some embodiments, the system may also take inputs (such as words or user states), transform those inputs into latent representations, and then generate content based on the latent representations using deep neural networks. In such embodiments, the system may be able to take currently presented content and generate new content using the currently presented content in a recursive manner. In some embodiments, the system may also be able to take the user state or a user input into the model to be transformed into latent representation to generate content. Some embodiments may be capable of generating music, images, stories, etc.

Content Embedded with Content Modification Processes

The content for use in the system described by FIG. 1A can include content modification processes. The content modification processes can, for example, be inherent to the content provided to the user. The content modification processes can include user triggered content modification processes (trigger user states), content triggered content modification processes (time codes), periodic modifications, or some combination thereof. The content modification processes can be purely inherent to the content (i.e., ignorant of external factors) or they can dynamically adjust based on, for example, user profile, historic data, prediction modes, or other factors. Content modification processes can adjust based on user response to prior content modification processes.

The content modification processes can include a trigger user state which can dictate what state the user needs to achieve to trigger the content modification process. For example, the trigger user state can include a brain state of the user (e.g., a pre-sleep state). The trigger user state can be determined by measuring bio-signals of the user. In some embodiments, the trigger user state can include a time code which can dictate at which point they can trigger. In other words, the trigger user state can include a user state and a time code at which the user state can trigger a modification. For example, if the content is a story, then the time codes may occur at natural pauses in the story to offer a change to induce a state change.

The content modification processes can include a modification which can modify a content element of the content. For example, the modification may increase or decrease volume, brightness, intensity, colour, contrast, or other characteristics of the content. In some embodiments, modifying the content element can include, for example, pausing the content. In some embodiments, modifying the content can include determining which content sample will follow a content sample that has concluded. In some embodiments, modifying the content may include transitioning between two parallel content channels (with or without bridging content). In some embodiments, when a trigger user state is achieved, the modification may not immediately be initiated (e.g., in a story, the content may wait until the end of a sentence to pause).

The content modification process can include an interval which can dictate how long the system will wait before querying the user state (e.g., to determine if a target user state was achieved or to initiate further content modifications). For example, where a state change is expected to occur promptly after a content modification, then the interval can be short. In some embodiments, where the state change is expected to occur a long time after the content modification, then the interval can be long. In some embodiments, the interval is the same length as the time it takes the content to modify (e.g., if volume will be decreased to volume level 30 over 5 s, then the interval may be 5 s). In some embodiments, the user state will in part define the interval (e.g., if the system determines that the individual is highly susceptible to a state change then the system may shorten a default interval). In some embodiments, the interval may be dictated by the length of a content sample (e.g., if the content transitioned to quiet whisper content sample, then the interval may be the length of the quiet whisper content sample). In some embodiments, the interval may be pre-defined.

In some embodiments, the content modification processes can include a target user state which can dictate what state the user needs to achieve to maintain the content modification process. For example, the target user state can include a brain state of the user (e.g., a sleep state). The target user state can be determined by measuring bio-signals of the user. In some embodiments, if the target user state is not achieved after an interval, then the system will completely reverse the modification. In some embodiments, if the target user state is not achieved after the interval, then the system can partially reverse the modification. In some embodiments, if the target user state is not achieved, then the system can further modify one or more content elements of the content. In some embodiments, if the target user state is achieved, then the system can further modify one or more content elements of the content (e.g., completely fade out the volume if the user falls asleep).

In some embodiments, the content modification processes can include a rate at which modifications will be applied to the content. Such rates may be fixed rates or other change profiles.

In some embodiments, the content modification processes can include a fail state wherein the content modification will continue to apply unless the user achieves the fail state.

In accordance with an aspect, there is provided a process or a use of time-coded content 702 to induce a change is state of at least one user by presenting the time-coded content 702 to the at least one user and using a bio-signal sensor. The time-coded content can include one or more content elements, one or more content modification processes 704. The content modification processes 704 can include a modification, a trigger, a target user state, and at least one interval. The content modification processes 704 can be configured to initiate the modification on detecting that the trigger is satisfied, modify one or more of the content elements based in part on the modification during the at least one interval, and modify one or more of the content elements based on a difference between a user state of the at least one user after the at least one interval, the target user state, and the modification.

In accordance with a further aspect, the trigger can include a trigger user state that the at least one user must satisfy and the modify one or more of the content elements based in part on the modification comprises modifying the one or more content element based in part on the user state.

In accordance with a further aspect, the trigger may include a time code in the content, and the modify one or more of the content elements based in part on the modification includes modifying one or more of the content elements at or after the time code. In some embodiments, the system may require that the user achieve a particular user state at a particular point in the content (or range of times). This may enable the system to initiate changes to the content in a seamless manner that can provide a consistent content experience to the user.

In accordance with a further aspect, the bio-signals of the at least one user may include bio-signals of a plurality of users, and the trigger user state or target user state may be based on each user of the plurality of users.

In accordance with a further aspect, the trigger user state may be determined based in part on a prediction model.

In accordance with a further aspect, the system further comprising a server configured to store the prediction model and provide the prediction model to the at least one computing device. The at least one computing device is configured to update the prediction model based on the difference between the user state of the at least one user after the at least one interval and the target user state.

In accordance with a further aspect, the prediction model comprises a neural network.

In accordance with a further aspect, the prediction model may be based in part on a user profile.

In accordance with a further aspect, the prediction model may be based in part on data from one or more other users.

In accordance with a further aspect, the one or more other users may share a characteristic with the at least one user.

In accordance with a further aspect, the at least one interval may be based in part on a current user state of the at least one user.

In accordance with a further aspect, the at least one interval is based in part on the content.

In accordance with a further aspect, the at least one interval is based in part on user input.

In accordance with a further aspect, the target user state is based in part on the content.

In accordance with a further aspect, the target user state may be based in part on input.

In accordance with a further aspect, the trigger user state is based in part on the content.

In accordance with a further aspect, the trigger user state may be based in part on input.

In accordance with a further aspect, modifying the one or more of the content elements is based in part on user input.

In accordance with a further aspect, at least one content modification process can be configured to determine a first user state of the at least one user using the bio-signals of the at least one user, apply a probe modification to one or more of the content elements provided to the at least one user, compute a difference between the first user state of the at least one user and the user state of the at least one user after a probe interval using the bio-signals of the at least one user, update at least one of the modification, the target user state, the trigger, and the at least one interval of one or more content modification processes based on a difference between the first user state and the user state of the at least one user after the probe interval.

In accordance with a further aspect, at least one content modification process is configured to determine a first user state of the at least one user using the bio-signals of the at least one user before a probe interval, compute a difference between the first user state of the at least one user before the probe interval and a user state of the at least one user after the probe interval using the bio-signals of the at least one user, update at least one of the target user state and the trigger user state based on the difference between the first user state and the user state after the probe interval.

In accordance with a further aspect, the content modification process can further comprise an exit user state and can be further configured to modify one or more of the content elements provided to the at least one user based on the difference between the user state of the at least one user during the at least one interval and the exit user state.

In accordance with a further aspect, the at least one bio-signal sensor may include at least one of EEG, EOG, EKG, EMG, PPG, heart rate, breath, sweat, gyroscopic, accelerometer, magnetometer, IMU, movement, vibration, sound, pulse wave amplitude, fNIRS, temperature, pressure, and electrodermal conductance sensors.

In accordance with a further aspect, the at least one user effector may include at least one of earphones, speakers, a display, a scent diffuser, a heater, a climate controller, a drug infuser or administrator, an electric stimulator, a medical device, a system to effect physical or chemical changes in the body, restraints, a mechanical device, a vibrotactile device, and a light.

In accordance with a further aspect, the content modification process may be further configured to modify auxiliary stimulus provided to the at least one user.

In accordance with a further aspect, the modify one or more of the content elements may include transitioning between one or more content samples.

In accordance with a further aspect, the modify one or more of the content elements includes pausing one or more of the content elements at time codes associated with natural breaks in the one or more content elements.

In accordance with a further aspect, the content modification process adjusts the interval based on natural breaks in the one or more of the content elements.

In accordance with a further aspect, the modify one or more of the content elements may include pausing one or more of the content elements.

In accordance with a further aspect, the time-coded content may include at least a first and a second time-coded content sample, and the modify one or more of the content elements may include transitioning between a first defined time-code of the first time-coded content sample to a second defined time-code of the second time-coded content sample.

In accordance with a further aspect, the first defined time code is based on natural pauses in the first time-coded content sample and the second defined time code is based on natural pauses in the second time-coded content sample.

In accordance with a further aspect, the second time-coded content sample is selected from a plurality of time-coded content samples based on at least on of the first time-coded content sample.

In accordance with a further aspect, the selection of the second time-coded content sample is based in part on a prediction model.

In accordance with a further aspect, the user state can include a brain state.

In accordance with a further aspect, the content elements can have modifications applied at a specific change profile.

In accordance with a further aspect, the trigger user state can include reaching a time code in the content.

### Exemplary Content Modification Profiles

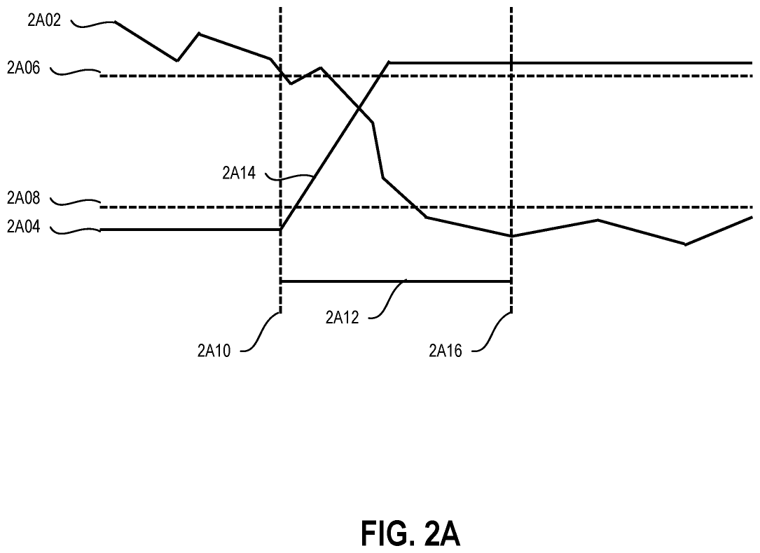

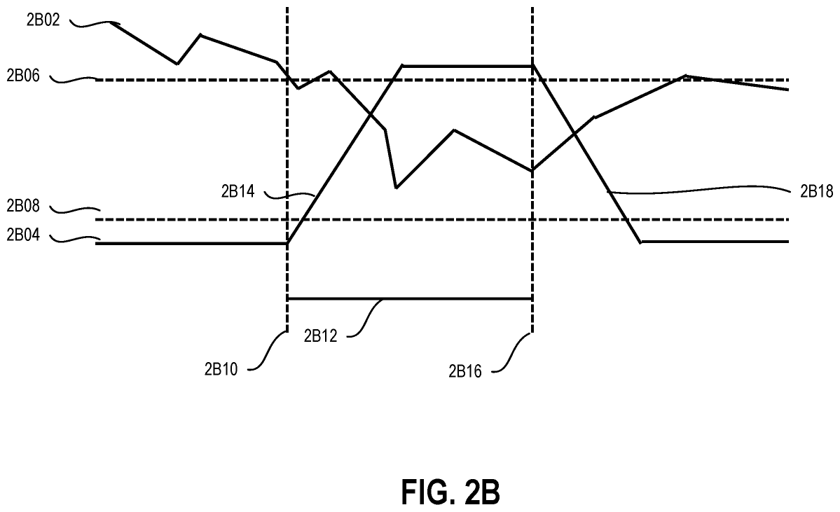

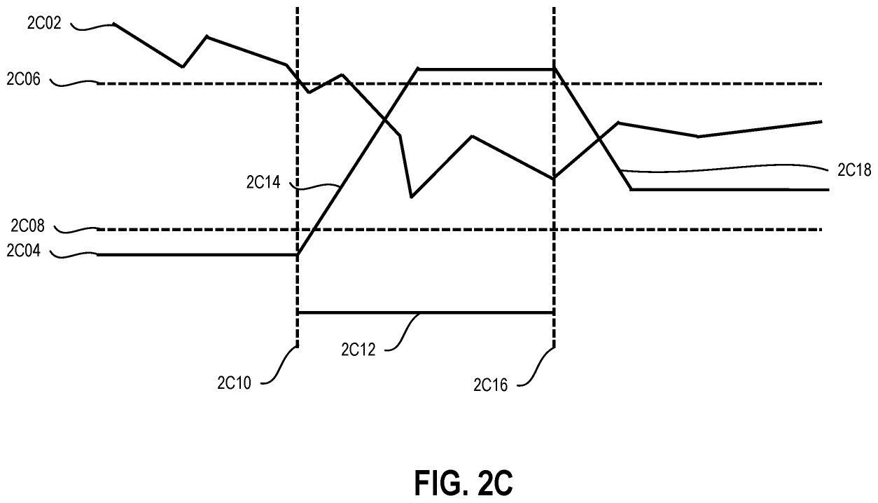

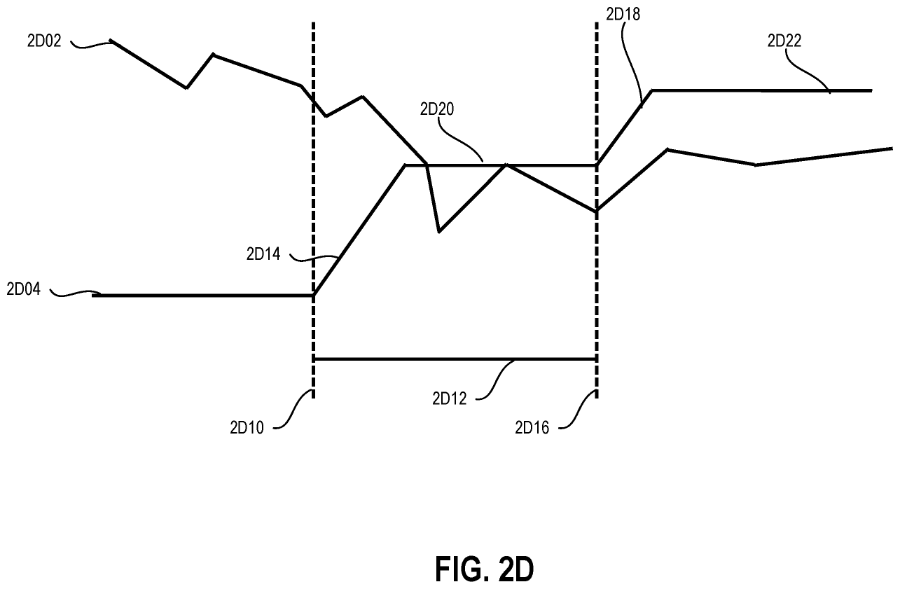

The following three figures FIG. 2A, FIG. 2B, and FIG. 2C, show example content modification processes based on a user trigger user state and further modified based on the achievement (or not) of a target user state.

FIG. 2A illustrates an example content modification process wherein the user achieved the target user state, according to some embodiments.

The brain state 2A02 is shown over time (with time moving forward from left to right). A level of content modification (e.g., amount of filtering or volume reduction) 2A04 is also plotted over time. The trigger user state 2A06 and target user state 2A08 are illustrated for convenience. The user is considered to be achieving the trigger user state 2A06 or target user state 2A08 if the user is below them. In an example embodiment, when the system detects that brain state 2A02 achieves the trigger user state 2A06 at time code 2A10, then the system sets interval 2A12 and initiates content modification 2A14. As is seen in the Figure, content modification 2A14 may take an amount of time and this time may be unrelated to interval 2A12. After the interval has elapsed at time code 2A16, the system detects the difference between the brain state 2A02 and the target user state 2A08. In this example, the user surpasses the target user state 2A08 and so the content modification is maintained.

FIG. 2B illustrates an example content modification process wherein the user did not achieve the target user state and the content is modified to reverse the first modification, according to some embodiments.

The brain state 2B02 is shown over time (with time moving forward from left to right). A level of content modification (e.g., amount of filtering or volume decrease) 2B04 is also plotted over time. The trigger user state 2B06 and target user state 2B08 are illustrated for convenience. The user is considered to be achieving the trigger user state 2B06 or target user state 2B08 if the user is below them. In an example embodiment, when the system detects that brain state 2B02 achieves the trigger user state 2B06 at time code 2B10, then the system sets interval 2B12 and initiates content modification 2B14. As is seen in the Figure, content modification 2B14 may take an amount of time and this time may be unrelated to interval 2B12. After the interval has elapsed at time code 2B16, then the system detects the difference between the brain state 2B02 and the target user state 2B08. In this example, the user did not achieve target user state 2B08 and so the computing device applies a subsequent content modification 2B18 to reverse modification 2B14.

FIG. 2C illustrates another example content modification process wherein the user did not achieve the target user state and the content is modified to partly reverse the first modification, according to some embodiments.

The brain state 2C02 is shown over time (with time moving forward from left to right). A level of content modification (e.g., amount of filtering or volume decrease) 2C04 is also plotted over time. The trigger user state 2C06 and target user state 2008 are illustrated for convenience. The user is considered to be achieving the trigger user state 2B06 or target user state 2B08 if the user is below them. In an example embodiment, when the system detects that brain state 2C02 achieves the trigger user state 2C06 at time code 2010, then the system sets interval 2C12 and initiates content modification 2014. As is seen in the Figure, content modification 2014 may take an amount of time and this time may be unrelated to interval 2C12. After the interval has elapsed at time code 2016, then the system detects the difference between the brain state 2C02 and the target user state 2C08. In this example, the user did not achieve target user state 2C08 and so the computing device applies a subsequent content modification 2C18 to partly reverse modification 2C14.

The following figure FIG. 2D show example content modification processes based on a periodically sampled user state.

FIG. 2D illustrates an example content modification process wherein final level of content modification is based on the user state, according to some embodiments.

The brain state 2D02 is shown over time (with time moving forward from left to right). The level of content modification (e.g., amount of filtering or volume decrease), including a first level of content modification 2D04, a second level of contend modification 2D20, and a third level of content modification 2D22, is also plotted over time. In an example embodiment, the system samples the user state at time code 2D10 and uses that user state to determine a second level of content modification 2D20. The system then changes the level of content modification from 2D04 to 2D20 at a particular rate 2D14 (in the Figure, a fixed rate, though other change profiles are conceived). Once the level of content modification reaches the second level 2D20, it remains at this level until the system samples the user state again at time code 2D16 after an interval 2D12. The user state at time code 2D16 can be used to determine another (here the third) level of content modification 2D22. The system then changes the level of content modification from 2D20 to 2D22 at a particular rate 2D18 (in the Figure, a fixed rate, though other change profiles are conceived). In some embodiments, the user state is continuously monitored, and specifically acted upon in this manner at specific time points 2D10 and 2D16 separated by interval 2D12. In some embodiments, the system can monitor to see if the user has reached an exit state in between these time points 2D10 and 2D16 wherein, for example, the content modification change is aborted or reversed or the system takes another action. In some embodiments, the rates at which content modification levels are changed (2D14 and 2D18) can be the same or different. In some embodiments, the rates 2D14 and 2D18 can be exponential, geometric, binary, perceptual, user specified, or some other rate change profile. In some embodiments, rates 2D14 and 2D18 can comprise complex rate changes better describes as a series of rate changes.

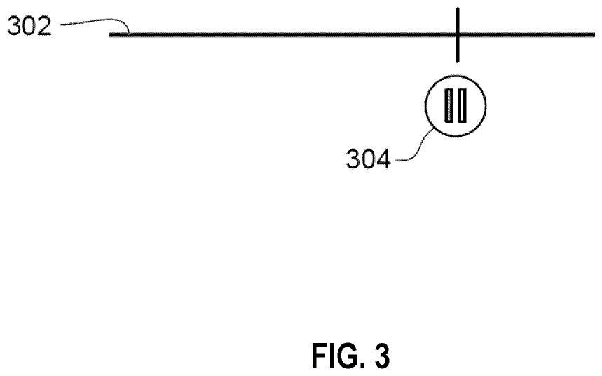

FIG. 3 illustrates an example content modification process involving a pause, according to some embodiments. In some embodiments, content 302 may be modified by inputting a pause at time code 304. For example, if the content is a story and the user is attempting to sleep, the content modification process may be triggered at that time code (e.g., the trigger user state may include a particular user state occurring at time code 304). If the trigger user state is achieved at time code 304, then the story may pause and the system may determine if the user falls asleep after an interval. If the user does not fall asleep, then the content may resume. In some embodiments, the story may resume at a decreased volume. Pauses may be input in stories at natural pauses in the story.

In some embodiments, the pauses may be a fixed length of time. For example, the pause could last 1 s if the system elects to take a pause (in this example, the natural pause in reading may be, for example, 0.2 s before moving to the next sentence). In some embodiments, different pauses could be coded to last different lengths (or relative lengths) of time. For example, pauses at the end of a sentence could be configured to last 0.5-2 s while those at the end of paragraphs could be configured to last 1-4 s dependent on the user state.

In some embodiments, the decision to pause the content and the length of that pause are dependent on the likelihood that doing so will induce a state change. For example, if the user is trying to fall asleep, the pauses may become longer and/or more frequent as the user becomes more tired. In some embodiments, the system may track how frequently the content is pausing (or otherwise factor past frequency of pausing into its determination of future probability of inducing a state change) to ensure that the system does not produce the opposite effect (i.e., driving the user away from, rather than towards, the desired user state by frequent pauses).

In accordance with a further aspect, the modify one or more of the content elements may include pausing one or more of the content elements 302. Pauses may occur at natural pauses in a narrative, for example.

In accordance with a further aspect, the modify one or more of the content elements comprises pausing one or more of the content elements at time codes associated with natural breaks in the one or more content elements. The system may be configured to receive or pre-process the content to identify natural pauses in the content (e.g., for narratives, natural pauses in speech, for music, natural low moments, etc.) and preference inserting pauses there.

In accordance with a further aspect, the computing device is further configured to adjust the interval based on natural breaks in the one or more of the content elements.

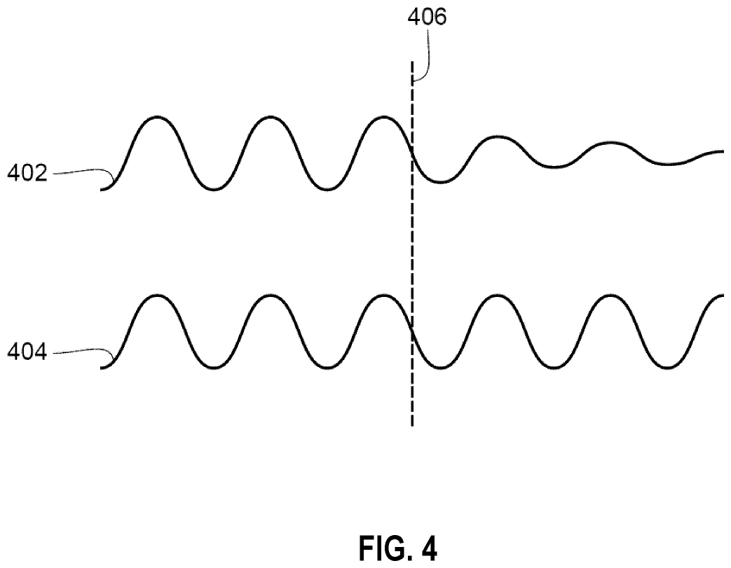

FIG. 4 illustrates an example content modification processes involving the modification of one content element, according to some embodiments. In some embodiments, different content elements may include different parts of an audio track. For example, content element 402 may include the vocals of a song and content element 404 may include the melody. When a content modification process is triggered at time code 406, then the system may reduce the volume of content element 402 (i.e., the vocals) while content element 404 (i.e., the melody) continues at the same volume. Other embodiments (i.e., where the content element is increases) are also conceived.

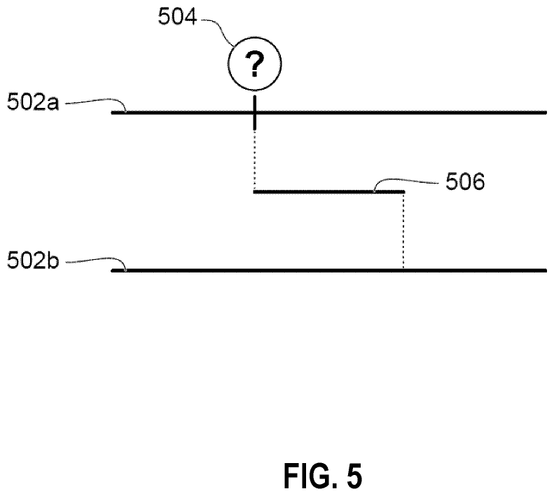

FIG. 5 illustrates an example time-coded content modification process, according to some embodiments. In some embodiments, the content may be, for example, a story that can transition between multiple tracks 502a and 502b. In this example, the system initiates a content modification at the time code 504. A user listening to track 502a may switch to transition track 506 and on completion, may be transferred to track 502b. Such transitions may be useful for different tracks that require a bridging track to produce a coherent content experience. Other, non-limiting examples of where this might be useful include in a naturescape. For example, track 502a represents a nearby thunderstorm and track 502b represents a distant thunderstorm, bridging track 506 may initiate at a specific time code of 502a to produce a coherent sounding distancing of the thunder storm (as opposed to merely modulating the volume of the thunderstorm or fading one track out while the other fades in-though both content modification processes are also possible in some embodiments). In some embodiments, the bridging track 506 may be configured to bridge if initiated at any time code rather than at a specific time code 504.

In accordance with a further aspect, the modify one or more of the content elements can include transitioning between one or more content samples 502. For example, the content may switch (or fade) between two parallel tracks.

In accordance with a further aspect, the content may include at least a first and a second time-coded content sample 502a and 502b and the modify one or more of the content elements may include transitioning between a first defined time code 504 of the first time-coded content sample 502a to a second defined time code of the second time-coded content sample 502b. In some embodiments, the story may truncate or abridge the story in order to arrive more quickly at the part where the user historically falls asleep. In some embodiments, there may be a bridging content sample 506.

In accordance with a further aspect, the first defined time code is based on natural pauses in the first time-coded content sample and the second defined time code is based on natural pauses in the second time-coded content sample. These time codes can be ascertained before or during content delivery.

In accordance with a further aspect, the second time-coded content sample is selected from a plurality of time-coded content samples based on at least on of the first time-coded content sample. The second time-coded content sample may be selected based on the narrative, thematic, or other flow with the first time-coded sample. In some embodiments, the second time-coded content sample may be procedurally generated from or based on the first time-coded content sample.

In accordance with a further aspect, the selection of the second time-coded content sample is based in part on a prediction model. The second time-coded content sample may be determined to assist in driving the user to the ultimate user state.

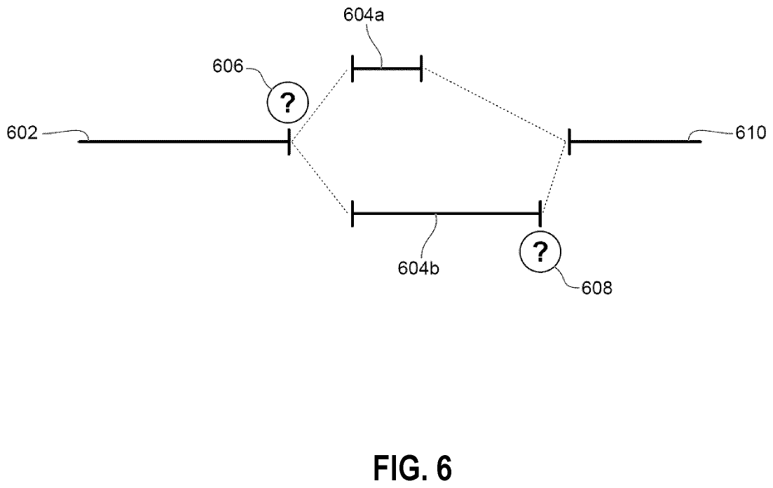

FIG. 6 illustrates example content stitched together from content samples, according to some embodiments.

In some embodiments, the content modifications may be time coded. For example, if content is a story, then it may be made up of several content samples. The initial sample 602 may represent a default story. At time code 606, the system may determine if a user has achieved a target user state and choose the next sample based on this determination. For example, if the user has not achieved a target user state, then the story may continue as normal with content sample 604a. However, if the user has reached the trigger user state, then the story may continue with modified content sample 604b which may include, for example, the same narrative as 604a, but read at a slower pace and in a whisper. In some example embodiments content sample 604b has its own point 608 wherein the system evaluates the user state to determine what path to follow. For example, point 608 can determine if the user has reached a target sleep state, and if so, the content may pause indefinitely as opposed to continuing with content sample 610.

In some embodiments some paths may converge again. In some embodiments, some content samples may represent a diversion within the content that is appropriate to bring the user through from more than one decision points, though it may only be appropriate to bring the user through it once (e.g., a sample introducing a new character may only happen the first time they are introduced in the story though they could be encountered in several different points within the story). In some embodiments, the content may in part or in whole be procedurally generated and content samples can be generated rather than selected based on a user state.

In some embodiments, the system is capable of remembering past content elements and the user reaction to them. In some embodiments, the system may preferentially choose content elements that the user is predicted to like. In some embodiments, the system is configured to continue presenting content elements that the user disliked in spite of them disliking it and query the user to see if they want to continue. In some embodiments, the user is a participant in content generation. In some embodiments, the system is configured to present the user with content they have not seen before. Such content generation can be thought of as interactive or conversational content generation between the user and the system.

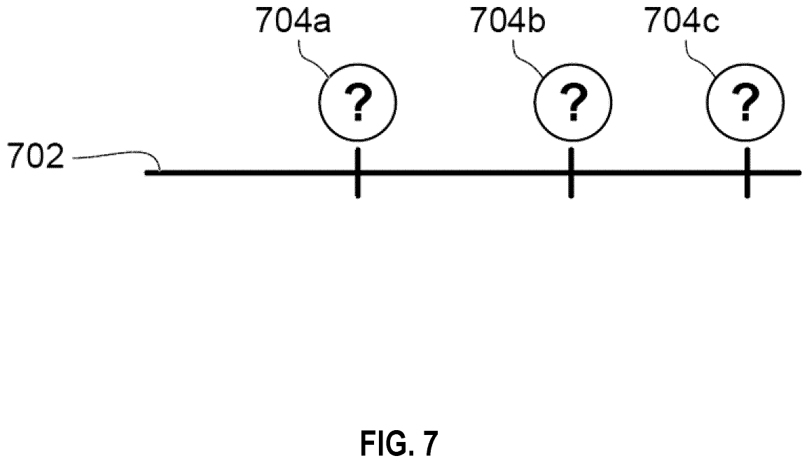

FIG. 7 illustrates example time-coded content with defined content modification process points, according to some embodiments.

In some embodiments, content 702 can include many time codes 704 (inclusive of 704a, 704b, and 704c) wherein each time code has an associated trigger (e.g., reaching the time code, achieving a trigger user state, or both). In some embodiments, the same content modification process can occur at each of the time codes 704. For example, content 702 may be a story and time codes 704 may correspond to natural breaks in the story. In this example, should the user achieve a pre-sleep state, then content 702 may pause at each of time codes 704 and wait to determine if the user will fall asleep. In some embodiments, time codes 704 can correspond to different content modification processes. In this example, time code 704a may decrease the volume if the user is in the trigger user state at time code 704a whereas content 702 may pause at time code 704b is the user is in the trigger user state, and 704c may decide on a subsequent content sample based on the user state.

In accordance with a further aspect, the content may include time-coded content 702, and the modify one or more of the content elements may be based in part on a current time code 704 in the time-coded content.

In accordance with a further aspect, the user state may include a brain state.

In accordance with a further aspect, the trigger user state can include reaching a time code in the content.

In accordance with a further aspect, the target brain state may include at least one of a sleep state, an awake state, an alert state, an arousal state, and a terror state. In some embodiments, the target user state may be a sleep state and the trigger user state may be a pre-sleep state. In these embodiments, softening or cutting the content in the pre-sleep trigger user state may induce a sleep state in the user. In some embodiments, the target user state may be an awake state and the trigger user state may be a pre-wakefulness state. In these embodiments, increasing the intensity or volume of the content when the user is in the pre-wakefulness state may induce a smooth rousing of the user. In some embodiments, for example when a user is trying to study, the target user state may be an alert state and the trigger user state may be a pre-flow state. In these embodiments, the content may provide engaging content to the user to clear the mind of other worries and when the system sees that the user is in the pre-flow state, the content may subtly reduce the audio fidelity or volume to possibly permit the user to focus on a task. In some embodiments, such as, for example, VR experiences, the target user state is a terror state and the trigger user state is a relaxed state. In these embodiments, the content may lull the user into a false sense of security and provide alarming content (such as the loud bang of a trash can falling over) when the system determines that the user feels secure. In these embodiments, the system may provide a non-threatening source of the alarming content if it determines the user did not enter a terror state (a cat knocking over a trash can) and may provide an enemy as the source of the alarming content where the user did enter a terror state (an enemy knocked over a trash can).

In some embodiments, the target user state may be different from the ultimate target user state. For example, if the ultimate user state is a sleep state, the system may bring the user through several intermediate target user states when executing its routine. In this example, it may first be necessary to engage the user's mind in the content to distract them from, for example, intrusive thoughts, before attempting to lull the user into a sleep state.

### Narrative Engine

The content modification types may apply individually or in some combination to content presented to a user. The type of modification may depend on the content. Content modifications may apply to some or all of the content presented to the user.

For example, the content presented to the user may comprise a narrative with procedurally generated background music. Content modification processes carried out on the background music may be partly independent from modifications (if any) carried out on the narrative. For example, the background music may vary its intensity (e.g., by modulating the speed at which notes are being played) based on periodically sampled user states. In some embodiments, content modification processes carried out on the background music may be partly dependent on content modification processes carried out on the narrative. For example, a decrease in background music intensity may coincide with a pause in the narrative triggered by a specific user state irrespective of whether the user state has been periodically sampled at that moment as part of the background music's periodic sampling.

The modification selector 19 can maintain a level of content coherence within the content presented to the user. For example, modification selector 19 may select content modification processes that are coherent with one another within the context of the content presented to the user. For example, the modification selector 19 can ensure that the volume level changes between different audio content elements are similar or partly dependent on one another. Modification selector 19 can provide visual content or music that matches the intensity of story provided to the user (procedurally generating high intensity music and/or visual effects when the story is energetic and bringing it down when not). Modification selector 19 can select content modification processes that do not call attention to themselves (e.g., not modifying the volume level repeatedly over a certain period of time which may call the user's attention to the volume level and not the content or achieving a target user state).

### Method of Implementation

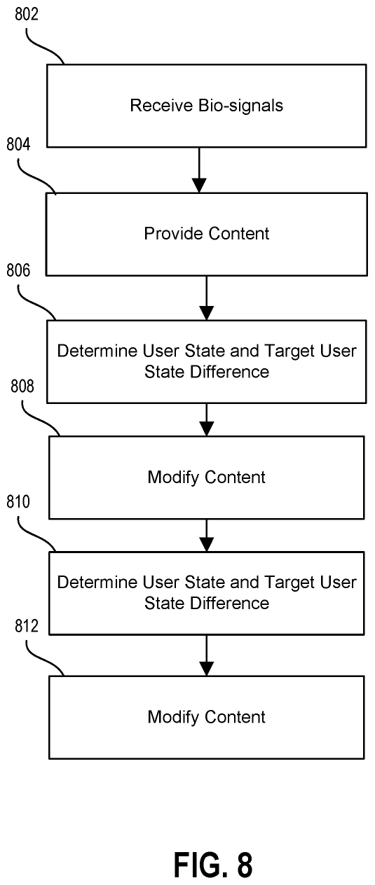

FIG. 8 illustrates the content modification process, according to some embodiments. Such a process can be implemented with, for example, system 100.

In accordance with an aspect, there is provided a method for achieving a target user state by modifying content elements provided to at least one user. The method may include receiving bio-signals of at least one user (802), providing content to the at least one user (804), the content comprising one or more content elements, computing a difference between a user state of the at least one user before an interval and the target user state using the bio-signals of the at least one user (806), modifying one or more of the content elements provided to the at least one user during the interval based on the difference between the user state of the at least one user before the interval and the target user state (808), computing a difference between the user state of the at least one user after an interval and the target user state using the bio-signals of the at least one user (810), and modifying one or more of the content elements provided to the at least one user after the interval based on the difference between the user state of the at least one user and the target user state (812).

In accordance with a further aspect, computing a difference between the user state of the at least one user before an interval and the target user state (806) includes determining that a trigger user state has been achieved using the bio-signals of the at least one user.

In accordance with a further aspect, the providing content to at least one user 802 may include providing content to a plurality of users, the user state may be based on the bio-signals of each user of the plurality of users.

In accordance with a further aspect, the user state may be determined based in part on a prediction model.

In accordance with a further aspect, the method further comprising updating the prediction model based on the difference between the user state of the at least one user after the interval and the target user state.

In accordance with a further aspect, the prediction model comprises a neural network.

In accordance with a further aspect, the prediction model may be based in part on a user profile.

In accordance with a further aspect, the prediction model may be based in part on data from one or more other users.

In accordance with a further aspect, the one or more other users may share a characteristic with the at least one user.

In accordance with a further aspect, the interval may be based in part on a current user state of the at least one user.

In accordance with a further aspect, the interval is based in part the content.

In accordance with a further aspect, the interval is based in part on user input.

In accordance with a further aspect, the target user state may be based in part on the content.

In accordance with a further aspect, the target user state may be based in part on input.

In accordance with a further aspect, the trigger user state may be based in part on content.

In accordance with a further aspect, the target user state may be based in part on input.

In accordance with a further aspect, modifying the one or more of the content elements (808 and/or 812) is based in part on user input.

In accordance with a further aspect, the method may further include determining a first user state of the at least one user using the bio-signals of the at least one user, applying a probe modification to one or more of the content elements provided to the at least one user, computing a difference between the first user state of the at least one user and the user state of the at least one user after a probe interval using the bio-signals of the at least one user, updating at least one of the target user state and the trigger user state based on the difference between the first user state and the user state after the probe interval.

In accordance with a further aspect, the method further including determining a first user state of the at least one user using the bio-signals of the at least one user before a probe interval, computing a difference between the first user state of the at least one user before the probe interval and a user state of the at least one user after the probe interval using the bio-signals of the at least one user, updating at least one of the target user state and the trigger user state based on the difference between the first user state and the user state after the probe interval.

In accordance with a further aspect, the method may further include computing a difference between the user state of the at least one user during the interval and an exit user state after using the bio-signals of the at least one user, and modifying one or more of the content elements provided to the at least one user based on the difference between the user state of the at least one user and the exit user state.

In accordance with a further aspect, the method may include modifying auxiliary stimulus provided to the at least one user.

In accordance with a further aspect, the modifying one or more of the content elements (808 and/or 812) may include transitioning between one or more content samples.

In accordance with a further aspect, the modifying one or more of the content elements (808 and/or 812) may include pausing one or more of the content elements.

In accordance with a further aspect, the modifying one or more of the content elements (808 and/or 812) includes pausing one or more of the content elements at time codes associated with natural breaks in the one or more content elements.

In accordance with a further aspect, the method further includes adjusting the interval based on natural breaks in the one or more of the content elements.

In accordance with a further aspect, the content may include at least a first and a second time-coded content sample, and the modifying one or more of the content elements (808 and/or 812) may include transitioning between a first defined time-code of the first time-coded content sample to a second defined time-code of the second time-coded content sample.

In accordance with a further aspect, the first defined time code is based on natural pauses in the first time-coded content sample and the second defined time code is based on natural pauses in the second time-coded content sample.

In accordance with a further aspect, the second time-coded content sample is selected from a plurality of time-coded content samples based on at least on of the first time-coded content sample.

In accordance with a further aspect, the selection of the second time-coded content sample is based in part on a prediction model.

In accordance with a further aspect, the content may include time-coded content, and the modifying one or more of the content elements (808 and/or 812) may be based in part on a current time code in the time-coded content.

In accordance with a further aspect, the user state includes a brain state.

In accordance with a further aspect, the content elements have modifications applied at a specific change profile.

In accordance with a further aspect, the trigger user state comprises reaching a time code in the content.

In accordance with an aspect there is provided a non-transient computer readable medium containing program instructions for causing a computer to perform any of the methods described herein.

In accordance with an aspect there is provided a hardware processor configured to assist in achieving a target brain state by processing bio-signals of at least one user captured by at least one bio-signal sensor and triggering at least one user effector to modify one or more of content elements. The hardware processor executing code stored in non-transitory memory to implement operations described in the description or drawings.

In accordance with an aspect there is provided a method to assist in achieving a target brain state by processing, using a hardware processor, bio-signals of at least one user captured by at least one bio-signal sensor and triggering at least one user effector to modify one or more of content elements, the method including steps described in the description or drawings.

### Generating Content Modification Processes

Referring again to FIG. 7, time-coded content 702 is provided. In some embodiments, the content modification processes 704 are input by the system based on feedback from a user. In some embodiments, the system is configured to randomly apply content modification processes (e.g., detect an initial user state at a time code, randomly modify the content, and detect a final user state after an arbitrary interval). The content can then be updated with this data to provide a content modification process based on the efficacy of the randomly applied content modification process.

In some embodiments, the content may be expertly trained and/or handcrafted (writing a song or story) to trigger certain content modification processes based on user states, thus providing optionality in the experience based on conditions. Machine learning, Artificial Intelligence, or other algorithmic processes can be used to optimize such expertly-crafted experiences. In some embodiments, a cost function may be used in machine learning that biases the system to provide the user with content modification processes that work well on other user.

In some embodiments the content may initially be totally random. In such embodiments, machine learning may be used to develop content modification processes that may work on the user de novo.

In some embodiments, the level of randomness permitted while training the system and generating the content may be a controlled boundary. For example, the system can apply different types of content modification process, but at specific time codes and learn which types of content modification process enhance the effect on the user. As another example, the type of content modification process may be fixed (or selected from a subset), but the system is configured to apply the content modification processes anywhere in the content to ascertain at which time codes the content modification processes have the biggest impact.

Content developed in this way can then be extracted with the embedded content modification processes therein and provided to other user. The systems used may be configured to calibrate these to other users (e.g., based on user profiles or preferences). In some embodiments, the systems may be configured to experience additional learning relevant to the other user. In some embodiments, the content with embedded content modification processes serves as a starting point to further randomly (or otherwise) modify the content for the other user and develop highly effective and personalized content modification processes.

In some embodiments, users can make inputs into the content and the content can be configured to adapt to these user preferences. For example, a user may be capable of disabling certain types of content modification processes. As another example, the user may be able to configure the time that content pauses or other intervals used by the system. In some embodiments, users can indicate preferences that are probabilistic in nature (e.g., they can reduce the likelihood of certain types of content modification processes occurring unless it meets a higher likelihood of inducing a desired user state change as compared to the general population on which the content was developed).

In example embodiments content might be developed to use a neural network to estimate a user's likelihood to fall asleep. The content may have an embedded frequency and length of pauses inserted into a story (i.e., the content) described as a probability function. The system determines whether to take a pause at sentence breaks is based on the likelihood that the user will undergo the desired change. The likelihood of inserting a pause can also be determined based on proximity in the story to the end (or to a section end), total listening time, what has induced the desired user state in the user in the past, etc.

Optimization techniques can be used to optimize content for the individual, for a population, or for a subset of the population (e.g., those with certain medical conditions). Optimization techniques can include gradient descent, back propagation, or random sampling method. Other optimization strategies are conceived.

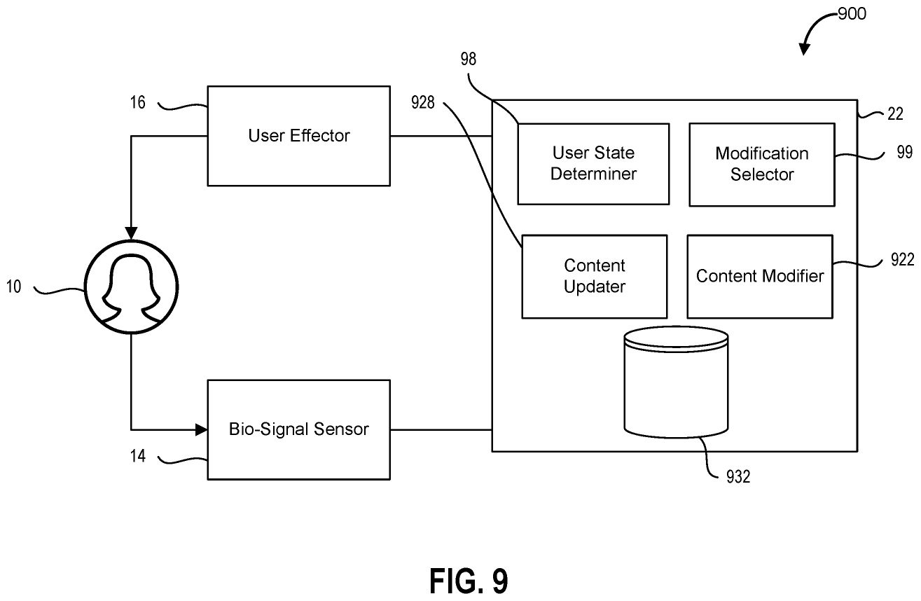

FIG. 9 illustrates a block schematic diagram of an example system that can update content, according to some embodiments.

System 900 can include a bio-signal sensor 14, computing device 22, and user effector 16. Bio-signal sensor 14 is capable of receiving bio-signals from user 10. User effector 16 can provide content to user 10. Computing device 22 can be in communication with bio-signal sensor 14 and user effector 16. In operation, computing device 22 can provide content to user 10 via user effector 16. Bio-signal sensor 14 can receive bio-signals from user 10 and provide them to computing device 22. Computing device 22 can determine user state changes in response to content modifications and can update the content to include new or modified content modification processes.

Computing device 22 includes a user state determiner 98, a content modifier 922, a modification selector 99, a content updater 928, and electronic datastore 932. In operation, computing device 22 can modify the content, determine a user reaction, and update the content using the user reaction. Computing device 22 can develop and map user engagement in content over time and by content element. Using computing device 22 may propagate content modification processes into a prediction model through, for example, a server.

User state determiner 98 may determine a state of user 10 using bio-signal sensor 14. In some embodiments, the determination made may be used to provide, for example, a trigger user state to a content modification process embedded within the content. For example, if a user is in a pre-sleep state and the content is muted and the user enters a sleep state, then the content may be updated to indicate that, should the user enter a pre-sleep state with similar characteristics, then muting the content may induce a sleep state in the user. The initial state may also include a time code (i.e., the user may need to achieve a trigger user state at or proximate to a time code in the content). In some embodiments, user state determiner 98 may determine the final user state of the user and use this to update a predicted final state of a user after a content modification process. The final state can be used to update the content to suggest that a user 10 may enter the final state if the user 10 achieves the initial state and system 900 modifies the content in a manner consistent with prior modifications as was determined.

Modification selector 99 can determine a content modification process to test the user with. Modification selector 99 can be configured to generate content modification processes to modify content in a manner that has a higher predicted probability of driving the user to a target user state than not modifying the content. In some embodiments, content modification processes can involve a specific type of content modification, a trigger user state for the content modification, a target user state for the modification, and optionally a fail condition (e.g., failure to reach the target user state after a pre-defined interval). In some embodiments, content modification processes can be configured to provide a pre-defined rate of content modifications (i.e., rate at which modification is applied to the content). In some embodiments, the content modification processes can include a rate of content modification application, a final level of content modification, and an interval, wherein the final level of content modification can be based in part on the user state. In some embodiments, content modification processes can involve selecting a path that the user takes through the content based on the user state. Modification selector 99 can be configured to track prior content modifications to generate content modification processes that can maintain coherence relative to each other.

Content modifier 922 can modify a content element delivered to user 10. Content modifier 922 can increase or decrease features of the content, insert pauses in a content element, and transition between content samples of the content elements. Content modifier 922 can make modifications to the content instantly or over a period of time. Modifications selector 99 cam control content modifier 922 directly or indirectly. Content modifier 922 can be configured to modify content separate and apart from content modifications determined by modifications selector 99 (e.g., it can be configured to filter high pitched noises from the content).

Content updater 928 updates the content to include a content modification process within the content. In some embodiments, the content modification process can include a trigger user state, a target user state, a modification, and an interval. The trigger user state may include a time code. The trigger user state can be updated using the initial state determined by user statement determiner 98. The interval and modification may be updated by the interval and modification used by modification selector 99. The target brain state may be updated using the final state determined by user state determiner 98. In some embodiments, the content modification process includes a method to determine a final content modification level (e.g., based on the user state determined using user state determiner 98), a rate to apply the content modification change, an interval, and optionally a time code in the content to query whether to make the content modification. In some embodiments, content modification processes include switching between different content samples. In such embodiments, the content modification process can include the initial user state prior to switching content samples and the content sample switched to.

Electronic datastore 932 is configured to store various data utilized by system 900 including, for example, data reflective of user state determiner 98, modification selector 99, content modifier 922, and content updater 928. Electronic datastore 932 may also store training data, model parameters, hyperparameters, and the like. Electronic datastore 932 may implement a conventional relational or object-oriented database, such as Microsoft SQL Server, Oracle, DB2, Sybase, Pervasive, MongoDB, NoSQL, or the like.

Some embodiments described herein can map the user engagement of the content. For example, in inserting content modification processes, the system can possibly predict which untested content modification processes are more likely to affect the user. For example, if the system consistently sees that decreases in volume at a particular time code in an audio track (e.g., a background conversation) can successfully induce a sleep state in a user, then the system may predict that decreasing the audio fidelity of that same track may also induce a sleep state. System 900 may also be implemented to determine what types of content modification processes may work across different types of content. For example, the system may be able to determine that sudden fade outs are effective at inducing a sleep state and may begin applying such modifications across different content.

In some embodiments, system 900 may be implemented to determine content specific, user specific, and content modification specific information. For example, system 900 may be able to ascertain what typical content modification processes or users (or a subset of users) respond well to or are driven towards a desired user state for a specific piece of content. As another example, system 900 may be able to ascertain what typical content and content modification processes are most effective for a specific user. As another example, system 900 may be able to ascertain what typical content and users (or a subset of users) respond well to or are driven towards a desired user state using specific content modification processes. The system 900 may be configured to further optimize variables associated with the content modification processes applied (i.e., trigger user states, rates of content change, intervals, etc.).

The system 900 can be used to generate content embedded with content modification processes (global content modification processes, time-coded content modification processes, content modifications processes configured to potentially trigger over a range of time codes, etc.). In some embodiments, the content embedded with content modification processes may then be used by another user to experience the content with no further optimizations. In some embodiments, the content embedded with content modification processes may use user profiles (or some other descriptor of the user, e.g., belonging to specific subsets of the population) to further adapt the content to the user. In some embodiments, the system may further optimize the content modification processes when provided to a second user after training (e.g., modifying the probability that specific content modification processes will trigger) based on the user's experience with that content.

Some embodiments can map time-coded content to induce a range of user states based on, for example, user preference. For example, the same music may be used for both waking and sleeping. The content may use different content modification processes embedded in the content itself to drive these differing ultimate user states. Some embodiments may incorporate content samples from other pieces of time-coded content to develop wholly unique content for user state manipulation. Some embodiments may use procedurally generated content to bring about user state changes and the procedure itself may be updated.

System 900 can, in some embodiments, work in tandem with systems 100, 100B, 100C, or 100D. For example, a system may be configured to deliver content and modify the content in response to a user achieving a trigger user state while also mapping user engagement with the time-coded content and generating new content modification processes. As such, alterations, combinations, and variations described for systems 100, 100B, 100C, and 100D can, to the extent applicable, apply to system 900.

In accordance with an aspect, there is provided a computer system 900 to develop time-coded content for achieving an ultimate user state by modifying content provided to the at least one user 10 in achieving an ultimate user state. The system 900 includes at least one computing device 22 in communication with at least one bio-signal sensor 14 and at least one user effector 16, the at least one bio-signal sensor 14 configured to measure bio-signals of at least one user 10, the at least one user effector 16 configured to provide time-coded content to the at least one user 10, wherein the time-coded content includes one or more content elements. The at least one computing device 22 can be configured to provide the time-coded content to the at least one user via the at least one user effector 16, determine an initial user state of the user at a time code using user state determiner 98, modify one or more of the content elements provided to the at least one user using content modifier 922, determine a final user state of the user after a test interval set by modification selector 99 using user state determiner 99, update the time-coded content to provide a content modification process including a target user state, an interval, a modification, and at least one of a time code and a trigger user state, wherein the trigger user state is based on the initial user state, the target user state is based on the final user state, the interval is based on the test interval, and the modification and the time code are based on the modify one or more of the content elements using content updater 928.

In accordance with a further aspect, the at least one computing device 22 can be further configured to determine another initial user state of the at least one user at another time code, wherein the another initial user state is determined with or after the final user state, modify one or more of the content elements provided to the at least one user, determine another final user state of the at least one user after another test interval, update the time-coded content to provide at least one more content modification process including a target user state, an interval, a modification, and at least one of a time code and a trigger user state, wherein the trigger user state is based on the another initial user state, the target user state is based on the another final user state, the interval is based on the another test interval, and the modification and the time code are based on the modify one or more of the content elements. In some embodiments, the content may be configured to bring the user through different target user states (i.e., intermediate target user states) before inducing an ultimate target user state. For example, to sleep a user may first need to be focused on the content (and distracted from other thoughts) before the system can effectively induce a sleep state.

In accordance with a further aspect, the time code can include at least one of a regular, a random, a pre-defined, an algorithmically defined, a user defined, and a triggered time code. In some embodiments the time code may include a range of time codes. In some embodiments the system 900 is configured to regularly test a content modification process. In some embodiments content modification processes are tested at random. In some embodiments, the content modification processes can have a time code pre-defined in the content, but the modification, interval, trigger, and target user state can all be randomized. In some embodiments the system can use historic data to algorithmically position content modification processes. In some embodiments the user (or another party) may define the time codes. In some embodiments, the time code can include a trigger user state wherein the initial brain state is selected for.

In accordance with a further aspect, the interval can include at least one of a regular, a random, a pre-defined, an algorithmically defined, a user defined, and a triggered interval. In some embodiments, the interval can be regularly set by the system. In some embodiments, the interval can be set at random. In some embodiments, the interval can be pre-defined while the time code and the modification are altered. In some embodiments the user (or another party) may define the intervals. In some embodiments the interval can be algorithmically determined based on historic data or other information.

In accordance with a further aspect, the modifications can include at least one of random, pre-defined, a user defined, and algorithmically defined modifications. In some embodiments, the modification can be random. In some embodiments, the modifications can be (in part or in whole) pre-defined while the time code and interval are varied. In some embodiments, the modifications can be algorithmically defined based on historic data or other information. Randomizing the modification may permit the system to stumble onto highly effective, but counterintuitive modifications, while pre-defining the modification may yield more consistent results. In some embodiments the user (or another party) may define the modifications. Algorithmically-defined modifications can also be algorithmically defined to modify the content in a manner wherein the outcome is highly uncertain which can provide the system with more information about the content or user.

In accordance with a further aspect, the content can be pre-processed to extract one or more content elements. In some embodiments, the system can accept raw content from an external source. In these embodiments, the system may be able to pre-process the data to extract content elements for individual manipulation. For example, for music content, the pre-processing may be able to separate the melody and vocal tracks. In another example, for story content, the pre-processing may be able to identify natural pauses in the story that may be conducive to inserted pauses.

In accordance with a further aspect, the at least one user effector 16 can be configured to provide content to a plurality of users 10 and the user state can be based on the bio-signals of each user of the plurality of users 10.

In accordance with a further aspect, the content modification processes can be based in part on a user profile.

In accordance with a further aspect, the interval can be based in part on a current user state of the at least one user 10.

In accordance with a further aspect, the content modification processes can further comprise an exit user state based on the final user state, the ultimate user state, and the modify one or more of the content elements.

In accordance with a further aspect, the at least one bio-signal sensor 14 can include at least one of EEG, EOG, EKG, EMG, PPG, heart rate, breath, sweat, gyroscopic, accelerometer, magnetometer, IMU, movement, vibration, sound, pulse wave amplitude, fNIRS, temperature, pressure, and electrodermal conductance sensors.

In accordance with a further aspect, the at least one user effector 16 can include at least one of earphones, speakers, a display, a scent diffuser, a heater, a climate controller, a drug infuser or administrator, an electric stimulator, a medical device, a system to effect physical or chemical changes in the body, restraints, a mechanical device, a vibrotactile device, and a light.

In accordance with a further aspect, the system 900 can further include one or more auxiliary effectors configured to provide stimulus to the at least one user and the computing device can be further configured to modify the stimulus provided to the at least one user 10 by the auxiliary effector.

In accordance with a further aspect, the modify one or more of the content elements can include transitioning between one or more content samples.

In accordance with a further aspect, the modify one or more of the content elements can include pausing one or more of the content elements.

In accordance with a further aspect, the modify one or more of the content elements includes pausing one or more of the content elements at time codes associated with natural breaks in the one or more content elements.

In accordance with a further aspect, the computing device is further configured to adjust the interval based on natural breaks in the one or more of the content elements.

In accordance with a further aspect, the time-coded content can include at least a first and a second time-coded content sample and the modify one or more of the content elements can include transitioning between a first defined time code of the first time-coded content sample to a second defined time code of the second time-coded content sample.

In accordance with a further aspect, the first defined time code is based on natural pauses in the first time-coded content sample and the second defined time code is based on natural pauses in the second time-coded content sample.

In accordance with a further aspect, the second time-coded content sample is selected from a plurality of time-coded content samples based on at least on of the first time-coded content sample.

In accordance with a further aspect, the user state can comprise a brain state.

In accordance with a further aspect, the content elements have modifications applied at a specific change profile.

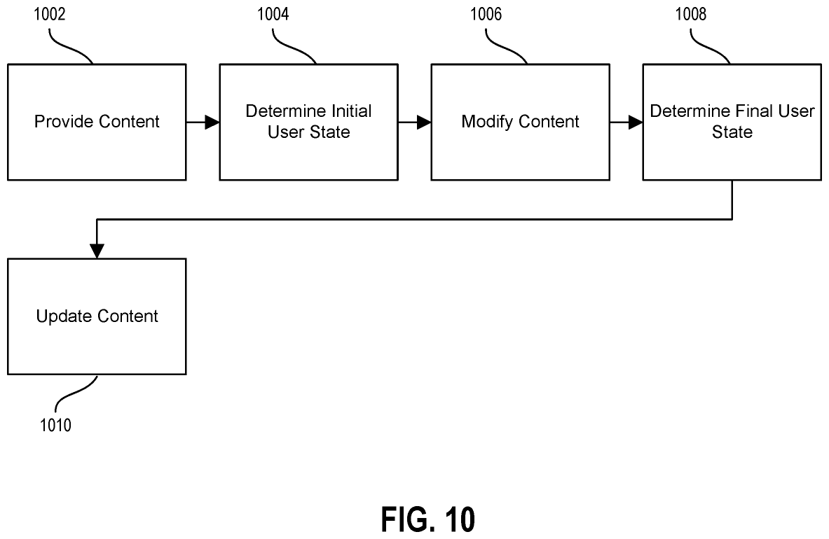

FIG. 10 illustrates an example content development process, according to some embodiments. Such a process can be implemented with, for example, system 900.

In accordance with an aspect, there is provided a method to develop time-coded content for achieving an ultimate user state by modifying content elements provided to at least one user. The method includes providing the time-coded content to the at least one user, the time-coded content including one or more content elements (1002), determining an initial user state of the at least one user at a time code using bio-signals of the at least one user (1004), modifying one or more of the content elements provided to the at least one user (1006), determining a final user state of the user after a test interval (1008), updating the time-coded content to provide a content modification process including a target user state, an interval, a modification, and at least one of a time code and a trigger user state, wherein the trigger user state is based on the initial user state, the target user state is based on the final user state, the interval is based on the test interval, and the modification and the time code are based on the modifying one or more of the content elements (1010).

In accordance with a further aspect, the method can further include determining another initial user state of the at least one user at another time code, wherein the another initial user state is determined with or after the final user state, modifying one or more of the content elements provided to the at least one user, determining another final user state of the at least one user after another test interval, and updating the time-coded content to provide at least one more content modification process including a target user state, an interval, a modification, and at least one of a time code and a trigger user state, wherein the trigger user state is based on the another initial user state, the target user state is based on the another final user state, the interval is based on the another test interval, and the modification and the time code are based on the modifying one or more of the content elements.

In accordance with a further aspect, the time code can include at least one of a regular, a random, a pre-defined, an algorithmically defined, a user defined, and a triggered time code.

In accordance with a further aspect, the interval can include at least one of a regular, a random, a pre-defined, a user defined, and an algorithmically defined interval.

In accordance with a further aspect, the modification can include at least one of a random, a pre-defined, a user defined, and an algorithmically defined modification.

In accordance with a further aspect, the time-coded content can be pre-processed to extract one or more content elements.

In accordance with a further aspect, the at least one user can include a plurality of users, the user state can be based on the bio-signals of each user of the plurality of users.

In accordance with a further aspect, the content modification processes can be based in part on a user profile.

In accordance with a further aspect, the interval can be based in part on a current user state of the at least one user.

In accordance with a further aspect, the content modification processes can further comprise an exit user state based on the final user state, the ultimate user state, and the modify one or more of the content elements.

In accordance with a further aspect, the method can further include modifying auxiliary stimulus provided to the at least one user.

In accordance with a further aspect, the modifying one or more of the content elements 1006 can include transitioning between one or more content samples.

In accordance with a further aspect, the modifying one or more of the content elements 1006 can include pausing one or more of the content elements.

In accordance with a further aspect, the modify one or more of the content elements 1006 comprises pausing one or more of the content elements at time codes associated with natural breaks in the one or more content elements.

In accordance with a further aspect, the computing device is further configured to adjust the interval based on natural breaks in the one or more of the content elements.

In accordance with a further aspect, the time-coded content can include at least a first and a second time-coded content sample and the modifying one or more of the content elements 1006 can include transitioning between a first defined time code of the first time-coded content sample to a second defined time code of the second time-coded content sample.

In accordance with a further aspect, the first defined time code is based on natural pauses in the first time-coded content sample and the second defined time code is based on natural pauses in the second time-coded content sample.

In accordance with a further aspect, the second time-coded content sample is selected from a plurality of time-coded content samples based on at least on of the first time-coded content sample.

In accordance with a further aspect, the user state can include a brain state.

In accordance with a further aspect, the content elements can have modifications applied at a specific change profile.

In accordance with an aspect there is provided a non-transient computer readable medium containing program instructions for causing a computer to perform any of the methods described herein.

### Mapping User States

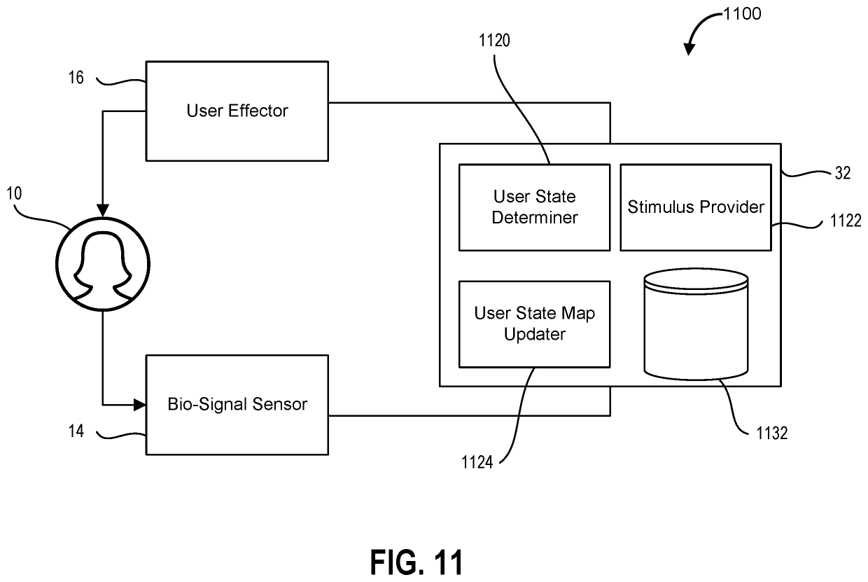

FIG. 11 illustrates a block schematic diagram of an example system that can map user states, according to some embodiments.

System 1100 can include a bio-signal sensor 14, computing device 32, and user effector 16. Bio-signal sensor 14 is capable of receiving bio-signals from user 10. User effector 16 can provide content to user 10. Computing device 32 can be in communication with bio-signal sensor 14 and user effector 16. In operation, computing device 32 can provide content to user 10 via user effector 16. Bio-signal sensor 14 can receive bio-signals from user 10 and provide them to computing device 32. Computing device 32 can determine user state changes in response to content modifications and can update the user state map.

Computing device 32 includes a user state determiner 1120, a stimulus provider 1122, a user state map updater 1124, and electronic datastore 1132. In operation, computing device 32 can modify the content, determine a user reaction, and update the user state map using the user reaction. Computing device 32 can develop and map user state transitions based on stimulus. Computing device 32 may propagate user state maps into a prediction model through, for example, a server.

User state determiner 1120 is capable of determining a user state before and after a stimulus is provided. The user state can include a brain state based on bio-signals. The user state can also take other information into account when making a user state determination.

Stimulus provider 1122 can provide stimulus to user 10. In some embodiments, the stimulus provided can include modifications to content that the user is receiving. In some embodiments, the stimulus can include modifications made to the content and an interval after the modification has been made. In some embodiments, the stimulus can include modification changes made at a specific rate. In some embodiments, the stimulus can include modifications made to the content at specified time codes or a range of time codes. In some embodiments, the stimulus can be presenting the user with certain content samples after other content samples have been presented. In some embodiments, the stimulus can include modifications made to probabilities used to generate procedural content or other variation to the procedural algorithm.

User state map updater 1124 updates the user state map. The user state map can include user state changes (i.e., user states before and after a stimulus is provided), a stimulus (or modification) that brought on the difference between the initial and final user states, and any interval between the stimulus and the final state. The user state map can be used to input content modification processes into raw content that are tailored to the user. For example, system 1100 may determine that fast content fade outs in a specific pre-sleep state are particularly effective in inducing a sleep state and so this content modification process can be applied to raw content never before seen by the user.

Electronic datastore 1132 is configured to store various data utilized by system 1100 including, for example, data reflective of user state determiner 1120, a stimulus provider 1122, a user state map updater 1124. Electronic datastore 1132 may also store training data, model parameters, hyperparameters, and the like. Electronic datastore 1132 may implement a conventional relational or object-oriented database, such as Microsoft SQL Server, Oracle, DB2, Sybase, Pervasive, MongoDB, NoSQL, or the like.

Some embodiments described herein can map the user states and more specifically transitions between the states. In doing so system 1100 may determine what types of content modifications are effective at inducing specific states in the user. Beyond this, system 1100 may be configured to determine a path of least resistance to reach an ultimate user state. For example, system 1100 may determine that user 10 can reach a sleep state more quickly if they are first deeply engrossed in content and system 1100 can develop a sleep induction procedure that attempts to first engross user 10 in the content and then induce sleep through a, for example, rapid content fade out.

In some embodiments, the content may not be analyzed prior to generating user state maps. In such embodiments, the content modification processes may be layered on top of the content. In some embodiments, unseen content may to be analyzed beforehand (or during) to ascertain likely content modification processes. Such embodiments may implement strict rules for how the content may be modified (e.g., the analysis identifies time codes at which it may input a pause and pauses are not permitted elsewhere in the content) or it may implement probabilistic changes to content modifications (e.g., the analysis provides a rough framework for approximate content modification time codes and types). In some embodiments, different analyses impact different content modification process types differently. In an example embodiment, a story (i.e., audio content reading a story) can be analyzed to determine natural times codes to pause (e.g., between sentences or paragraphs) or change to a new story.

In some embodiments, where the content is a narrative, the user state maps can be used to associate one or more content samples (part of a story) with one another. In this way, the system may be appropriate for use in generating a library of different content samples that can invoke similar user state transitions. The user state maps can help generate a story space in which a narrative operates. The story space can comprise a plurality of content samples (procedurally generated or otherwise) that the user can explore (consciously, subconsciously, or otherwise). The content samples can be cataloged and associated in terms of narrative elements (e.g., concrete plot details to avoid plot holes) and/or user state map elements (e.g., state transitions to be induced by engaging in the content). This may allow a user to be exposed to narratively new content that the system may still predict to induce desired state changes in the user.

The exploration of the story space may be based on moment-to-moment or longer term user states. The exploration may also include elements of conscious user choice. In some embodiments, the narrative is delivered and uses active (conscious) user participation to explore initially and as the narrative goes on, more and more decisions in the narrative are based on the user states (e.g., subconscious user states) as the user drifts into sleep.

Further analyses can be carried out that layer in additional content to enhance the user experience or preferentially drive the user to a desired user state. For example, audiobooks may have background music layered in. In some embodiments, the speed or volume of the story being read may be altered based on the themes in the book (e.g., determined using machine learning, e.g., keyword analysis).

System 1100 can, in some embodiments, work in with systems 100, 100B, 100C, 100D, or 900. For example, a system may be configured to deliver content and modify the content in response to a user achieving a trigger user state while also mapping user states and associating the user states with the user profile or updating a prediction model with the user states. As such, alterations, combinations, and variations described for systems 100, 100B, 100C, 100D, or 900 can, to the extent applicable, apply to system 1100. In particular, embodiments described above for systems 100, 100B, 100C, 100D, or 900 can apply to embodiments of system 1100.

In accordance with an aspect, there is provided a computer system 1100 to map user states. The system 1100 including at least one computing device 32 in communication with at least one bio-signal sensor 14 and at least one user effector 16. The at least one bio-signal sensor 14 configured to measure bio-signals of at least one user 10. The at least one user effector 16 configured to provide stimulus to the at least one user 10. The at least one computing device 32 configured to determine an initial user state using user state determiner 1120, provide stimulus to the at least one user using stimulus provider 1122, determine a final user state using user state determiner 1120, update a user state map using the stimulus, initial user state, final user state using user state map updater 1124.

In accordance with a further aspect, the user state map can be updated using a time code at which the stimulus was provided to the at least one user.

In accordance with a further aspect, the computing device 32 may be further configured to receive user input on the initial user state or the final user state that describes the state. For example, if the user is attempting to reach a happy state, then the system may query them about their contentment level in particular states. Such an example could be used for therapeutic purposes. In some embodiments, the users may label the desirability, the emotional or cognitive experience, the level of focus, the associative/dissociative experience, the embodiment, the degree of sensory experience, the spirituality, the fear reaction (e.g., fight or flight), the stability, the vulnerability, the connectivity (isolation or level of connection), and the restlessness of the state.

In accordance with a further aspect, the computing device 32 may be further configured to provide stimulus to the at least one user that is predicted to direct the at least one user into desirable user states. Once system 1100 determines desirable user states (based on the system's goals) then it can attempt to modify content delivered to the user to induce said desirable user state changes.

In accordance with a further aspect, the determine the final user state using the user state determiner 1120 may include determining the final user state after an interval set by an interval setter. In such embodiments, the interval may permit the stimulus or content modification to take full effect on the user.

In accordance with a further aspect, the stimulus may include modification of content presented to the at least one user 10, and the update a user state map may include generating content modification process that includes a trigger user state based on the initial user state, a target user state based on the final user state, and a modification based on the modification of content presented to the at least one user. In some embodiments, effective content modification processes can be determined for a particular user or in the aggregate.

In accordance with a further aspect, the computing device 32 may be further configured to induce the target user state by initiating the content modification process when the at least one user achieves the trigger user state. System 1100 may be configured to use the user state map to map out trigger and target user states to direct a user to an ultimate user state. In some embodiments, system 1100 may be configured to find a ‘path of least resistance’ through the state map to achieve an ultimate user state.

In accordance with a further aspect, the user state map may be associated with a user profile of the at least one user 10 and the system 1100 may be further be configured to apply the content modification process to other content when the user achieves the trigger user state. The state map may be uniquely associated with the user 10. The state map may be subsequently studies to determine aggregate or average or general state maps. The state map may also be used to modify subsequent content to induce desirable state changes (e.g., to induce sleep in fresh content).

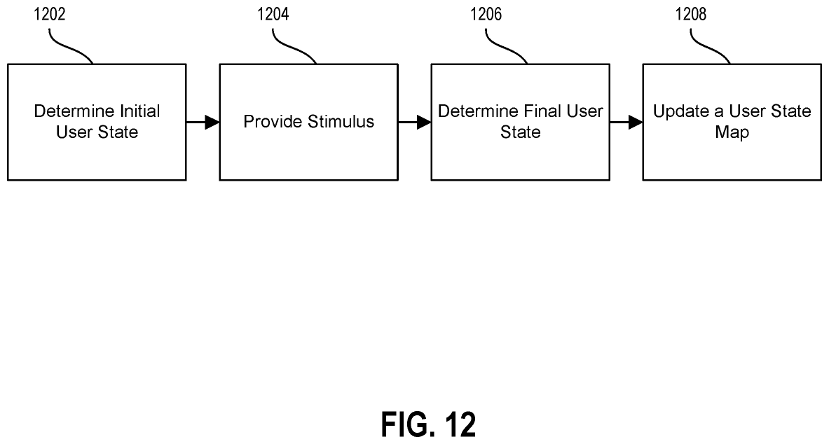

FIG. 12 illustrates the an example user state mapping process, according to some embodiments. Such a process can be implemented with, for example, system 1100.

In accordance with an aspect, there is provided a method to map user states, the method including determining an initial user state (1202), providing stimulus to the at least one user (1204), determining a final user state (1206), updating a user state map using the stimulus, initial user state, final user state (1208).

In accordance with a further aspect, updating the user state map 1208 includes updating the user state map using a time code at which the stimulus was provided to the at least one user.

In accordance with a further aspect, the method may further include receiving user input on the initial user state or the final user state that describes the desirability of the state.

In accordance with a further aspect, the method may further include providing stimulus to the at least one user predicted to direct the at least one user into desirable states.

In accordance with a further aspect, the determining the final user state may include determining the final user state after an interval.

In accordance with a further aspect, the stimulus may include modification of content presented to the at least one user, and the updating a user state map 1208 may include generating content modification process that may include a trigger user state based on the initial user state, a target user state based on the final user state, and a modification based on the modification of content presented to the at least one user.

In accordance with a further aspect, the method may further include inducing the target user state by initiating the content modification process when the at least one user achieves the trigger user state.

In accordance with a further aspect, the method may further comprise associating the user state map with a user profile of the at least one user, and applying the content modification process to other content when the user achieves the trigger user state.

In accordance with an aspect there is provided a non-transient computer readable medium containing program instructions for causing a computer to perform any of the methods described herein.

### Implementation Details to Enable Other Signals to be Used to Determine a User State

In some embodiments, it may be more convenient for the system to determine a user state (e.g., a brain state) based on other signals rather than conventional bio-signals. In such embodiments, the system may be configured to determine the user state (e.g., brain state) based on other signals by initially using bio-signals to determine the user state and associating the user state with other signals. Such embodiments may allow the user to omit wearing bio-signal sensors after the system has been trained.

In particular, for certain user states, the bio-signal sensors may be cumbersome to wear and as such, providing an alternative means to determine the user state (e.g., brain state of the user) may be beneficial. In some embodiments, such as sleeping, it may not be optimal to consistently require the user to wear a sensor.

Some embodiments are configured to train a system to measure and detect other signals to determine a user state. The other signals can be used to supplement or to replace the bio-signal data. For example, detecting the ambient temperature that is hot may provide the system with an alternative explanation for profuse sweating by the user. In another example, the system may be configured to determine that a fast typing speed indicates a focus state.

In the following embodiments, reference is made to bio-signal-sensors and other signal sensors. By way of example, the bio-signal-sensor can be a sensor which may be capable of directly measuring the body. Another example signal sensor may be a sensor which can capture sensor data or signals that the system can be trained to use to infer user states (e.g., brain states).

As the system learns to associate sensor data and signals with certain user states (e.g., brain states), different types of sensor data and signals can be used similarly to bio-signals to determine the user state (in particular for implementations described above). Accordingly, the system can make a prediction based on different types of sensor data and signals similar to bio-signals in order to infer user states.

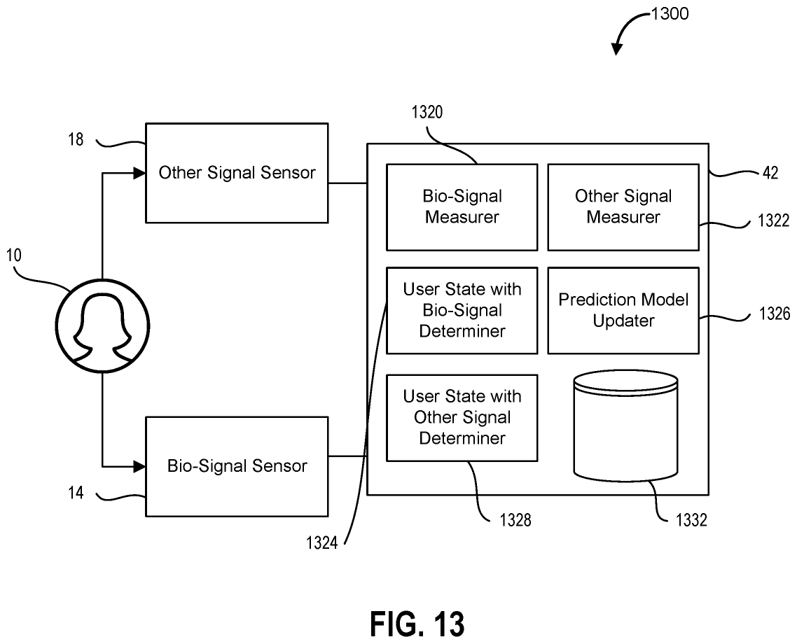

FIG. 13 illustrates a block schematic diagram of an example system that can associate other signals with user states, according to some embodiments.

System 1300 can include a bio-signal sensor 14, computing device 42, and other signal sensor 15. Bio-signal sensor 14 is capable of receiving bio-signals from user 10. Other signal sensor 15 is capable of receiving other signals from user 10. Computing device 42 can be in communication with bio-signal sensor 14 and other signal sensor 15. In operation, computing device 42 can determine user states (e.g., brain states) based on the bio-signal sensors and use those determinations to update a prediction model that permits the system to determine user states based on other signals.

Computing device 42 includes a bio-signal measurer 1320, other signal measurer 1322, user state with bio-signal determiner 1324, prediction model updater 1326, user state with other signal determiner 1328, and electronic datastore 1332. In operation, computing device 42 can update and develop a prediction model to assist system 1300 to produce possibly more accurate user state predictions or predictions based on different or less data.

Bio-signal measurer 1320 is capable of measuring bio-signals of the user 10. It can do this using bio-signal sensor 14.

Other signal measurer 1322 is capable of measuring other signals of the user 10. It can do this using other signal sensor 15.

User state with bio-signal determiner 1324 can determine the user state (e.g., a brain state) of the user using the bio-signals of the user 10. This user state may be based on a prediction model which may be downloaded from, for example, a server or developed by system 1300 (e.g., stored on electronic datastore 1332).

Prediction model updater 1326 can be used to provide additional known data to the prediction model and to update the other signals associated with the known user states. The prediction model can, for example, include a neural network. The prediction model can be general or trained with data arising from the specific user 10. The prediction model can in some embodiments facilitate transfer learning or provide a system capable of recognizing contextual information to complement bio-signal data and infer user states. Such a prediction model may permit the system 1300 or other systems making use of the prediction model trained with system 1300 to be more portable or otherwise require fewer signal sensors to determine a user state.

User state with other signal determiner 1328 may use the prediction model to predict a user state based on other signals. This component can make use of the prediction model updated by the prediction model updater 1326 and other signals received from the other signal sensor.

Electronic datastore 1332 is configured to store various data utilized by system 1100 including, for example, data reflective of a bio-signal measurer 1320, other signal measurer 1322, user state with bio-signal determiner 1324, prediction model updater 1326, and user state with other signal determiner 1328. Electronic datastore 1332 may also store training data, model parameters, hyperparameters, and the like. Electronic datastore 1332 may implement a conventional relational or object-oriented database, such as Microsoft SQL Server, Oracle, DB2, Sybase, Pervasive, MongoDB, NoSQL, or the like.

Some embodiments can effectively generate a prediction model capable of relying more heavily on other signals to determine a user state. This may permit the user to omit wearing some or all of the bio-signal sensors in favour of using other sensors.

System 1300 can, in some embodiments, work with systems 100, 100B, 100C, 100D, 900, or 1100. For example, a system may be trained with system 1300 to determine, for example, the user state based in whole or in part on other signals and systems 100, 100B, 100C, 100D, 900, or 1100 can be configured to use other signal data to determine the user state. In a manner, the other signals can be thought of as bio-signals for the purposes of systems 100, 100B, 100C, 100D, 900, or 1100, or other variations. As such, alterations, combinations, and variations described for systems 100, 100B, 100C, 100D, 900, or 1100 can, to the extent applicable, apply to system 1100. In particular, embodiments described above for systems 100, 100B, 100C, 100D, 900, or 1100 can apply to embodiments of system 1300.

In accordance with an aspect, there is provided a computer system 1300 to detect a user state of at least one user 10. The system including at least one computing device 42 in communication with at least one bio-signal sensor 14, and at least one other signal sensor 18. The at least one bio-signal sensor 14 configured to measure bio-signals of at least one user 10. The at least one other signal sensor 18 configured to measure other signals of the at least one user 10. The at least one computing device 42 configured to measure the bio-signals of the at least one user using bio-signal measurer 1320, measure the other signals of the at least one user using other signal measurer 1322, determine a user state of the at least one user using the measured bio-signals and a prediction model using user state with bio-signal determiner 1324, update the prediction model with the determined user state and the measured other signals of the at least one user using prediction model updater 1326, determine the user state of the at least one user using the measured other signals and the updated prediction model using the user state with other signal determiner 1328.

In accordance with a further aspect, the system 1300 may be further configured to perform an action based on the user state determined using the measured other signals and the updated prediction model. For example, in operation, the system 1300 may be configured to deliver content to the user 10 and modify the content when a trigger user state is achieved to induce a target user state.

In accordance with a further aspect, the system 1300 further comprising a server configured to store the prediction model and provide the prediction model to the at least one computing device 42. The at least one computing device 42 is configured to update the prediction model on the server. In some embodiments, the prediction model can be made available on multiple devices and can inform (i.e., provide data for) a more generalized prediction model.

In accordance with a further aspect, the prediction model comprises a neural network.

In accordance with a further aspect, the other signals may include at least one of a typing speed, a temperature preference, ambient noise, a user objective, a location, ambient temperature, an activity type, a social context, a user preferences, self-reported user data, dietary information, exercise level, activities, dream journals, emotional reactivity, behavioural data, content consumed, contextual signals, search history, and social media activity. Some embodiments may make use of differing signals. Typing speed may indicate productivity and focus. Temperature preference or ambient temperature may indicate comfort level. Ambient noise may indicate focus. User objective may indicate target user state. Location may indicate user state information (e.g., if the user is at work, they may be stressed). Activity type may provide indirect bio-information. Social context may indicate a level of anxiety. Social context may provide information about how crowded a room is which may indicate user stress. User preferences may reflect user self-reported states. Dietary information may indicate a user's comfort. Exercise level may indicate frustration. Activities may provide contextual information about the user state. Dream journals may offer insight into baseline user states (e.g., pre-occupation with work stress may manifest in nightmares about work). Emotional reactivity may determine user susceptibility to state changes. Behavioural data may offer mood indications (e.g., keeping the blinds drawn may indicate depression). Social media activity may reveal current preoccupations and extent thereof.

Dietary information and exercise level may be determined from health apps. Health apps may be able to provide both bio-signal data (e.g., heart rate) and other signals for the system. Health apps may also provide contextual social information.

Contextual signals can include signals which are on their own innocuous, but that the system has observed indicate a user state or a state change in certain contexts. For example, the system may be configured to detect user movement in bed (e.g., rolling over) and after observation determines that the user rolling over may indicate that the user has entered a sleep state (or has a probability of having done so). In further uses, the system may detect and/or rely on the rolling over signal to indicate a sleep state. Other contextual signals may include the coincident of two signals (e.g., the user yawning while reading in low light indicating they may want to initiate sleep transition content modification processes).

The environment in which the user sleeps may also provide other signals such as context of sleep, whether the user is sleeping with another individual, other context surrounding sleep (e.g., ambient noise or content consumed before sleep or stated user objectives to encounter certain dreams).

In accordance with a further aspect, the other signals may include bio-signals or behaviours of other individuals. In some embodiments, the system may be configured to determine internal user states based on context cues offered by other individuals when interacting with the user. In some embodiments, the system may be configured to sense the user state based on individual states of other individuals. Such embodiments may be highly effective when determining the state of individuals that are emotionally close to the user.

In an example, the user may be a part of a ‘dream club’ (wherein the users may experience a shared dream experience). In this example, some of the signals may be provided by receiving feedback from the group in real time. In this example, pre- or post-user interactions with other individuals may be used to inform the user state.

In accordance with a further aspect, the prediction model may be based in part on a user profile.

In accordance with a further aspect, the prediction model may be based in part on data from one or more other users.

In accordance with a further aspect, the one or more other users may share a characteristic with the at least one user.

In accordance with a further aspect, the at least one bio-signal sensor may comprise at least one of EEG, EOG, EKG, EMG, PPG, heart rate, breath, sweat, gyroscopic, accelerometer, magnetometer, IMU, movement, vibration, sound, pulse wave amplitude, fNIRS, temperature, pressure, and electrodermal conductance sensors.

In accordance with a further aspect, the user state can include a brain state.

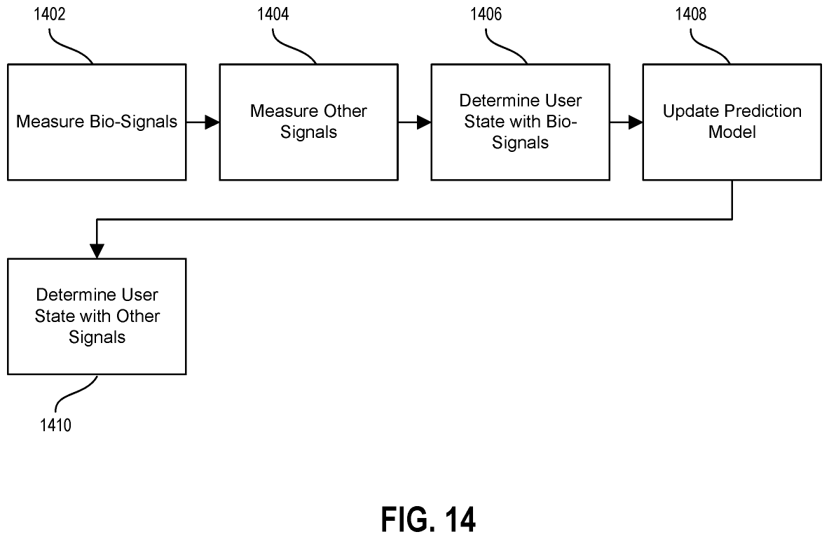

FIG. 14 illustrates the an example other signal and user state association process, according to some embodiments. Such a process can be implemented with, for example, system 1300.

In accordance with an aspect, there is provided a method to detect a user state of at least one user. The method including measuring bio-signals of at least one user (1402), measuring other signals of the at least one user (1404), determining a user state of the at least one user using the measured bio-signals and a prediction model (1406), updating the prediction model with the determined user state and the measured other signals of the at least one user (1408), determining the user state of the at least one user using the measured other signals and the updated prediction model (1410).

In accordance with a further aspect, the method may further include performing an action based on the user state determined using the measured other signals and the updated prediction model.

In accordance with a further aspect, the prediction model includes a neural network.

In accordance with a further aspect, the other signals may include at least one of a typing speed, a temperature preference, ambient noise, a user objective, a location, ambient temperature, an activity type, a social context, a user preferences, self-reported user data, dietary information, exercise level, activities, dream journals, emotional reactivity, behavioural data, content consumed, contextual signals, search history, and social media activity.

In accordance with a further aspect, the other signals may include bio-signals or behaviours of other individuals.

In accordance with a further aspect, the prediction model may be based in part on a user profile.

In accordance with a further aspect, the prediction model may be based in part on data from one or more other users.

In accordance with a further aspect, the one or more other users share a characteristic with the at least one user.

In accordance with a further aspect, the user state can include a brain state.

In accordance with an aspect there is provided a non-transient computer readable medium containing program instructions for causing a computer to perform any of the methods described herein.

### Optional Uses

Optionally, the systems, methods, or devices of the present invention may be used to implement aspects of the systems and methods described in PCT Patent Application No. PCT/CA2021/051079, filed 30 Jul. 2021, the entirety of which is incorporated by reference herein. Accordingly, training of the system may make use of the self supervised learning paradigms described therein. Accordingly, the systems, methods, or devices described herein may be interoperable with a system for training a neural network to classify bio-signal data by updating trainable parameters of the neural network. The system has a memory and a training computing apparatus. The memory is configured to store training bio-signal data from one or more subjects. The training bio-signal data includes labeled training bio-signal data and unlabeled training bio-signal data. The training computing apparatus is configured to receive the training bio-signal data from memory, define one or more sets of time windows within the training bio-signal data, each set including a first anchor window and a sampled window, for at least one set of the one or more sets, determine a determined set representation based in part on the relative position of the first anchor window and the sampled window, extract a feature representation of the first anchor window and a feature representation of the sampled window using an embedder neural network, aggregate the feature representations using a contrastive module, and predict a predicted set representation using the aggregated feature representations, update trainable parameters of the embedder neural network to minimize a difference between the determined set representation of the at least one set and the predicted set representation of the at least one set, and label the unlabeled training bio-signal data using a classifier, the labeled training bio-signal data, and the embedder neural network. The set representation denotes likely label correspondence between the first anchor window and the sampled window.

Optionally, the systems, methods, or devices of the present invention may be used to implement aspects of the systems and methods described in PCT Patent Application No. PCT/CA2020/051672, filed 4 Dec. 2020, the entirety of which is incorporated by reference herein. Accordingly, the systems, methods, or devices described herein may be interoperable with a wearable device that has a flexible and extendable body configured to encircle a portion of a body of a user, an electronics module with a concave space between two ends, each end attachable to the flexible and extendable body with a flexible retention mount to allow rotation of the flexible and extendable body relative to the electronics module and to transfer tension force from the flexible and extendable body to the electronics module, and a bio-signal sensor disposed on the flexible and extendable body to contact at least part of the body of the user and to receive bio-signals from the user.

Optionally, the systems, methods, or devices of the present invention may be used to implement aspects of the systems and methods described in U.S. patent application Ser. No. 16/858,093, filed 24 Apr. 2020, the entirety of which is incorporated by reference herein. Accordingly, the systems, methods, or devices described herein may be interoperable with a computer-implemented method for brain modelling. The method comprising receiving time-coded bio-signal data associated with a user, receiving time-coded stimulus event data, projecting the time-coded bio-signal data into a lower dimensioned feature space, extracting features from the lower dimensioned feature space that correspond to time codes of the time-coded stimulus event data to identify a brain response, generating a training data set for the brain response using the features, training a brain model using the training set using a processor that modifies parameters of the brain model stored on the memory, the brain model unique to the user, generating a brain state prediction for the user output from the trained brain model, using a processor that accesses the trained brain model stored in memory, and using a processor that automatically computes similarity metrics of the brain model as compared to other user data and inputting the brain state prediction to a feedback model to determine a feedback stimulus for the user, wherein the feedback model is associated with a target brain state.

Optionally, the systems, methods, or devices of the present invention may be used to implement aspects of the systems and methods described in U.S. patent application Ser. No. 16/206,488, filed 30 Nov. 2018, the entirety of which is incorporated by reference herein. Accordingly, the systems, methods, or devices described herein may be interoperable with a wearable device to wear on a head of a user. The device including a flexible band generally shaped to correspond to the user's head, the band having at least a front portion to contact at least part of a frontal region of the user's head, a rear portion to contact at least part of an occipital region of the user's head, and at least one side portion extending between the front portion and the rear portion to contact at least part of an auricular region of the user's head, a deformable earpiece connected to the at least one side portion. The deformable earpiece including conductive material to provide at least one bio-signal sensor to contact at least part of the auricular region of the user's head. At least one additional bio-signal sensor disposed on the band to receive bio-signals from the user.

Optionally, the systems, methods, or devices of the present invention may be used to implement aspects of the systems and methods described in U.S. patent application Ser. No. 16/959,833, filed 4 Jan. 2019, the entirety of which is incorporated by reference herein. Accordingly, the systems, methods, or devices described herein may be interoperable with a wearable system for determining at least one movement property. The wearable system includes a head-mounted device including at least one movement sensor, a processor connected to the head-mounted device, and a display connected to the processor. The processor includes a medium having instructions stored data that when executed cause the processor to obtain sensor data from the at least one movement sensor, determine at least one movement property based on the obtained sensor data, and display the at least one movement property on the display.

Optionally, the systems, methods, or devices of the present invention may be used to implement aspects of the systems and methods described in U.S. patent application Ser. No. 14/368,333, filed 6 Jan. 2014, the entirety of which is incorporated by reference herein. Accordingly, the systems, methods, or devices described herein may be interoperable with a system including at least one computing device. The at least on computing device including at least one processor and at least one non-transitory computer readable medium storing computer processing instructions, and at least one bio-signal sensor in communication with the at least one computing device. Upon execution of the computer processing instructions by the at least one processor, the at least one computing device is configured to execute at least one brain state guidance routine comprising at least one brain state guidance objective, present at least one brain state guidance indication at the at least one computing device for presentation to at least one user, in accordance with the executed at least one brain state guidance routine, receive bio-signal data of the at least one user from the at least one bio-signal sensor, at least one of the at least one bio-signal sensor comprising at least one brainwave sensor, and the received bio-signal data comprising at least brainwave data of the at least one user, measure performance of the at least one user relative to at least one brain state guidance objective corresponding to the at least one brain state guidance routine at least partly by analyzing the received bio-signal data, and update the presented at least one brain state guidance indication based at least partly on the measured performance.

Optionally, the systems, methods, or devices of the present invention may be used to implement aspects of the systems and methods described in U.S. Pat. No. 10,452,144, filed 30 May 2018, the entirety of which is incorporated by reference herein. Accordingly, the systems, methods, or devices described herein may be interoperable with a mediated reality device. The mediated reality device including an input device and a wearable computing device with a bio-signal sensor, a display to provide an interactive mediated reality environment for a user, and a display isolator. The bio-signal sensor receives bio-signal data from the user. The bio-signal sensor including a brainwave sensor, wherein the bio-signal sensor is embedded in the display isolator, wherein the bio-signal sensor includes a soft, deformable user-contacting surface.

Optionally, the systems, methods, or devices of the present invention may be used to implement aspects of the systems and methods described in U.S. Pat. No. 10,120,413, filed 11 Sep. 2015, the entirety of which is incorporated by reference herein. Accordingly, the systems, methods, or devices described herein may be interoperable with a training apparatus that has an input device and a wearable computing device with a bio-signal sensor and a display to provide an interactive virtual reality (“VR”) environment for a user. The bio-signal sensor receives bio-signal data from the user. The user interacts with content that is presented in the VR environment. The user interactions and bio-signal data are scored with a user state score and a performance scored. Feedback is given to the user based on the scores in furtherance of training. The feedback may update the VR environment and may trigger additional VR events to continue training.

Optionally, the systems, methods, or devices of the present invention may be used to implement aspects of the systems and methods described in U.S. Pat. No. 9,563,273, filed 6 Jun. 2011, the entirety of which is incorporated by reference herein. Accordingly, the systems, methods, or devices described herein may be interoperable with a brainwave actuated apparatus. The brainwave actuated apparatus including a brainwave sensor for outputting a brainwave signal, an effector responsive to an input signal, and a controller operatively connected to an output of said brainwave sensor and a control input to said effector. The controller is adapted to determine characteristics of a brainwave signal output by said brainwave sensor and based on said characteristics, derive a control signal to output to said effector.

Optionally, the systems, methods, or devices of the present invention may be used to implement aspects of the systems and methods described in U.S. Pat. No. 10,321,842, filed 22 Apr. 2015, the entirety of which is incorporated by reference herein. Accordingly, the systems, methods, or devices described herein may be interoperable with an intelligent music system. The system may have at least one bio-signal sensor configured to capture bio-signal sensor data from at least one user. The system may have an input receiver configured to receive music data and the bio-signal sensor data, the music data and the bio-signal sensor data being temporally defined such that the music data corresponds temporally to at least a portion of the bio-signal sensor data. The system may have at least one processor configured to provide a music processor to segment the music data into a plurality of time epochs of music, each epoch of music linked to a time stamp, a sonic feature extractor to, for each epoch of music, extract a set of sonic features, a biological feature extractor to extract, for each epoch of music, a set of biological features from the bio-signal sensor data using the time stamp for the respective epoch of music, a metadata extractor to extract metadata from the music data, a user feature extractor to extract a set of user attributes from the music data and the bio-signal sensor data, the user attributes comprising one or more user actions taken during playback of the music data, a machine learning engine to transform the set of sonic features, the set of biological features, the set of metadata, and the set of user attributes into, for each epoch of music, a set of categories that the respective epoch belongs to using one or more predictive models to predict a user reaction of music, and a music recommendation engine configured to provide at least one music recommendation based on the set of labels or classes.

Optionally, the systems, methods, or devices of the present invention may be used to implement aspects of the systems and methods described in U.S. Pat. No. 9,867,571, filed 6 Jan. 2015, the entirety of which is incorporated by reference herein. Accordingly, the systems, methods, or devices described herein may be interoperable with a wearable apparatus for wearing on a head of a user. The apparatus including a band assembly including an outer band member including outer band ends joined by a curved outer band portion of a curve generally shaped to correspond to the user's forehead, an inner band member including inner band ends joined by a curved inner band portion of a curve generally shaped to correspond to the user's forehead, the inner band member is attached to the outer band member at least by each inner band respectively attached to a respective one of the outer band ends, at least one brainwave sensor disposed inwardly along the curved inner band portion, and biasing means disposed on the curved inner band portion at least at the at least one brainwave sensor to urge the at least one brainwave sensor towards the user's forehead when worn by the user.

Optionally, the systems, methods, or devices of the present invention may be used to implement aspects of the systems and methods described in U.S. Pat. No. 10,365,716, filed 17 Mar. 2014, the entirety of which is incorporated by reference herein. Accordingly, the systems, methods, or devices described herein may be interoperable with a method, performed by a wearable computing device including at least one bio-signal measuring sensor. The at least one bio-signal measuring sensor including at least one brainwave sensor. The method including acquiring at least one bio-signal measurement from a user using the at least one bio-signal measuring sensor, the at least one bio-signal measurement including at least one brainwave state measurement, processing the at least one bio-signal measurement, including at least the at least one brainwave state measurement, in accordance with a profile associated with the user, determining a correspondence between the processed at least one bio-signal measurement and at least one predefined device control action, and in accordance with the correspondence determination, controlling operation of at least one component of the wearable computing device.

Optionally, the systems, methods, or devices of the present invention may be used to implement aspects of the systems and methods described in U.S. Pat. No. 9,983,670, filed 16 Sep. 2013, the entirety of which is incorporated by reference herein. Accordingly, the systems, methods, or devices described herein may be interoperable with a computer network implemented system for improving the operation of one or more biofeedback computer systems. The system includes an intelligent bio-signal processing system that is operable to capture bio-signal data and in addition optionally non-bio-signal data, and analyze the bio-signal data and non-bio-signal data, if any, so as to extract one or more features related to at least one individual interacting with the biofeedback computer system, classify the individual based on the features by establishing one or more brain wave interaction profiles for the individual for improving the interaction of the individual with the one or more biofeedback computer systems, and initiate the storage of the brain wave interaction profiles to a database, and access one or more machine learning components or processes for further improving the interaction of the individual with the one or more biofeedback computer systems by updating automatically the brain wave interaction profiles based on detecting one or more defined interactions between the individual and the one or more of the biofeedback computer systems.

Optionally, the systems, methods, or devices of the present invention may be used to implement aspects of the systems and methods described in U.S. Pat. No. 10,009,644, filed 4 Dec. 2013, the entirety of which is incorporated by reference herein. Accordingly, the systems, methods, or devices described herein may be interoperable with a system including at least one computing device, at least one biological-signal (bio-signal) sensor in communication with the at least one computing device, at least one user input device in communication with the at least one computing device. The at least one computing device is configured to present digital content at the at least one computing device for presentation to at least one user, receive bio-signal data of the at least one user from the at least one bio-signal sensor, at least one of the at least one bio-signal sensor comprising a brainwave sensor, and the received bio-signal data comprising at least brainwave data of the at least one user, and modify presentation of the digital content at the at least one computing device based at least partly on the received bio-signal data, at least one presentation modification rule associated with the presented digital content, and at least one presentation control command received from the at least one user input device. The presentation modification rule may be derived from a profile which can exist locally on the at least one computing device or on a remote computer server or servers, which may co-operate to implement a cloud platform. The profile may be user-specific. The user profile may include historical bio-signal data, analyzed and classified bio-signal data, and user demographic information and preferences. Accordingly, the user profile may represent or comprise a bio-signal interaction classification profile.

### Example Use—Falling Asleep

In some embodiments, the systems, methods and devices described herein may be configured to induce a sleep state in the user. In embodiments in which the system may be configured to trigger a content modification process based on a user state, the target user state can be a sleep state and the content may be a story or music (audio). In an example embodiment, the user may be wearing smart headphones which are capable of delivering audio to the user and measuring the user's bio-signals. The headphones may have an onboard computer capable of directing the headphones to deliver content and to measure the bio-signals of the user.

In some embodiments, one of the content modification processes may be triggered by a user state. In such embodiments, the trigger user state may be one where the user is on the verge of sleep. Being a partially unconscious process, a system capable of unobtrusively cuing sleep at the right moment may be more effective than similar processes attempted by an individual. In this example embodiment, the system may deliver audio to the user while the user is trying to fall asleep. The audio can initially be presented to the user in an unmodified form. Once the user's user state is at or near the trigger user state, then the system may implement a content modification process wherein the audio volume decreases to 50% over a 20 s period. This may cue the user to enter the sleep state. The interval may be set to, for example, 30 s. After the 30 s has elapsed, the system will determine if the user has entered a sleep state and if the user has, then the headphones continue to decrease the volume to silence. However if the user has not entered any of these states or has become more conscious, then the system may increase the volume over a 20 s period. The final volume of the content may be based on the user's present state. For example, if the user did not enter a sleep state, but is still semi-conscious, then the final volume level may be quiet (e.g., 70% of original volume).

In some embodiments, one of the content modification processes may periodically sample the user state and trigger based on the user's present user state. For example, the system may sample the user state at least every 30 s and based on the assessment at that 30 s mark. The system may set a final content modification level based on the user state. In some embodiments, the system can set the final content modification level on the probability that the user is in or out of a user state (e.g., set volume to 50% because the user has a 50% probability of not being asleep). The system may then be configured to change the level of content modification applied to the content at a fixed rate (such as four percentage points per second) or otherwise pre-defined rate until it reaches the final content modification level (i.e., 50%). After 30 s have elapsed (i.e., the periodic interval), the system can again sample the user state and again set another final content modification level based on that user state.

In some embodiments, as the user uses the system, the system may learn what types of content modification the user responds well to and how long a change in user state generally takes the user. For example, some users may be particularly susceptible to falling asleep if the global volume of the music fades out over a 180 s period, while other users may be susceptible to falling asleep if the vocals are quickly cut from the content and the melody fades over a much longer period.

Some users may experience state changes quickly once they experience their cue while others may take much longer to experience a state change once they receive their cue. For example, the system may wait a much shorter interval to determine if the user has entered their target sleep state if the user typically enters into the target sleep or semi-consciousness state quickly.

In some embodiments, the user state may be periodically sampled. In such embodiments, the system may determine a final level of content modification based on the periodically sampled user state and apply these modifications at a fixed rate until the final level of content medication is achieve. In such embodiments, the final level of content modification may be based on the probability that the user is in an awake state (e.g., if the user has a 50% probability of being in an awake state, then the final level of content modification may be determined to be 50% of, for example, the volume). There may be an interval between the periodic sampling of the user state and the final level of content modification may be updated after the interval.

### Example Use—Waking Up

Some embodiments of the described systems, methods, and devices may be capable of rousing a user from sleep. In these embodiments, the user's target user state may be awake. In some embodiments, the system can trigger content modification processes based on the user achieving a trigger user state. The trigger user state may be a pre-awake state. For example, when the system determines it is time to rouse the user, the system may present the user with energetic music. The system may monitor the user's state to determine when the music brings the user to a pre-awake state and therefore susceptible to being awoken. When the system determines that the user has entered the pre-awake trigger user state, then the system may modify the content to, for example, emphasize an alarm sound that plays along to the rhythm of the music. If after 30 s the user has not roused, then the system may remove this alarm sound and resume playing the energetic music without this modification. However if after 30 s the user has roused and become awake, then the system may modify the content again to remove all content provided to the user (i.e., turn the alarm off and return to silence and permit the user to go about their morning routine).

In some embodiments, the content provided to the user may induce a change in sleep state to gradually rouse the user from one sleep state to the next. In these embodiments, the system is capable of providing content to the user and modifying the content to bring the user through, for example, several target sleep states (of varying consciousness levels). The content can be provided to induce the state changes in the user from a deep sleep through an awake state rather than, necessarily, waiting on the user to enter a predefined state before providing content or modifications thereof. In some examples, the content change its target user state if a user fails to achieve a target user state from a previous content modification process (i.e., if the system doesn't succeed with one modification, it may try another).

In some embodiments the user may be able to pre-program specific content modification rules. For example, the energetic music delivered to the user to rouse them may be selected specifically because it is energetic, but once the user has roused, the system may modify the content to deliver news to the user with light music playing in the background while the user goes about their morning routine.

In some embodiments, the system may be configured to redirect the emotional energy of the user arising from previous dream energy (e.g., reground them). In some embodiments, the user can be exposed to musical content in a minor key and when the user rises, the minor key can change to a major key. In some embodiments, the system can be configured to provide content to the user that is both familiar and positive when the user rouses to provide an emotionally positive start to the day. In some embodiments, the system can provide the user with content to set up a pay off for when the user rouses. For example, the system may be configured to present an orchestral piece wherein the energy builds as the user rouses and crescendos when the user reaches the ultimate awake state. As another example, the content may provide a soundscape of a user's favourite movie to prime the user and when the user wakes up, the content modifies to present the moment in the movie that provides the user with energetic release (e.g., the moment that gives the user goosebumps).

### Example Use—Lucid Dreaming

Some embodiments of the described systems, methods, and devices may be capable of bringing the user into a lucid dreaming state. In these embodiments, the user's target user state may be a partially awake state. The system may be configured to provide energetic content (e.g., higher volume, more engaging content than that provided to make them sleep) to the user to slightly rouse the user if it determines that they are in too deep of sleep. The system can be configured to detect if a user is being roused too much and provide content to lull them back to sleep. In such embodiments the system may be configured to monitor the user's semi-conscious internal state and modify the content according to those states. In this way the content provided to the user which may form the basis of their dream, may be altered by the user's semi-conscious thoughts and the user may be provided with indirect control over their dreams to encourage a lucid dreaming state.

In further embodiments, the system can be configured to query the user to see if they are in a lucid dream state. For example the user may be asked directly if they are lucidly dreaming and to respond the system may ask them to bring about a specific internal state. The system may determine that the user is lucidly dreaming once the user conjures this state. In other embodiments, the user may be asked to move slightly (e.g., eye movement) which the system can pick up on to determine that the user is lucid.

In some embodiments, the system may query the user to see what they are dreaming about and based on the user response, the system may be configured to take its next action based on the user's belief that they are dreaming.

Once the user achieves a lucid dreaming state, the system may be configured to stop providing content to the user or to provide content that is heavily based on the user's state to further enhance the lucidity of the dream (rather than detract from it by influencing it with content not fully under user control).

### Example Use—Studying

Some embodiments of the described systems, methods, and devices may be capable of cueing the user to enter a flow state. In these embodiments, the user's target user state may be a flow state. In an example embodiment, the user may be provided with soundscape content such as a the sound of a train in a rain storm.

In this example embodiment, the soundscape may begin as a highly dynamic soundscape with many content elements such as the rattling of a train, the train whistle, the intensity of the rain, and the presence of thunder. Each of these elements can be modified individually. When the user initially implements the system, the content may be highly engaging to distract the user from sounds in their physical environment. As the user focuses on their task, their mind may enter a focus state. At this point, the system may modify the content to be more melodic and trancelike, for example, by pausing the train whistle and thunder sound effect and modifying the train rattling and rain soundtracks to be more consistent. If after two minutes the user has entered the flow state, then the modifications to the soundscape may be maintained. If however, the user has not entered a flow state after the two minute interval has elapsed, then the system may modify the content to restore the train whistle sound effect, for example.

In some embodiments, the system may periodically query the user state and change the content elements based on those queries.

### Example—Learning a Language

In some embodiments, the content modification can include modifying the language in which the content is presented.

In some embodiments, the content provided may also be intended to educate or achieve another goal with the user. In some embodiments, the user can receive instruction in a foreign language (i.e., instruction in how to speak said language) and as the user enters a sleep state, the content may modify to induce a sleep state and to continue to expose the user to the foreign language. For example, as the user falls asleep, the content may change from language instruction to low level conversations in the foreign language or phonemes spoken in said language. The low level (e.g., low volume) can induce a sleep state, while the language spoken can continue to expose the user to the foreign language. This example system may return to the instruction when the user rouses.

### Example Use—Smart Cars

Some embodiments of the described systems, methods, and devices may be capable of cueing the user to enter an alert state because they are, for example, driving a car. In these embodiments, the user's target user state may be an alert state.

In an example embodiment, the user may be driving their car and would like to maintain an alert level so that they are paying attention to the road. The system may expose the user to energetic music. When the system detects that the user is entering a focus state, then the system may modify the music, for example, by enhancing the base. If the user enters into an alert state, then the system can maintain this enhancement. If the user does not enter the alert state, then the system can, for example, decrease the base to cue the user up for another base enhancement which may cue the user to enter an alert state. The system may be further configured to make loud sounds (similar to the operation of rumble strips on roads) to bring the user back to the target focused state if the car detects that the user is about to be distracted. In the event that the user does not achieve the target focused state, then the system can further increase the level and intensity of the alarms.

### Example Use—Inducing Fear in Horror

Some embodiments of the described systems, methods, and devices may be capable of cueing the user to become fearful, for example, for entertainment. In these embodiments, the user's target user state may be a terror state.

For example, if the intended experience is an effective ‘jump scare’, then the content modification process may be triggered by a trigger user state that is a relaxed state. In this embodiment, the system may deliver soothing and relaxing content to the user to lull them into a false sense of security. Once the system detects that the user is relaxed, then the system may modify the content to introduce a sudden loud sound to scare the user. If after a short interval, the system determines that the user has entered the target tense state, then the system may further modify the content and proceed to deliver greater degree of horror content. If, instead the system determines that the user did not enter the target tense state, then the system may resume providing relaxing content to the user to lull them back into a false sense of security.

In another example, the intended experience may be one of constant tension and heightened terror. In these embodiments, the content delivered may be calibrated to keep the user on edge and when they are most susceptible to a scare (i.e., when they are jumpy), the system may rapidly modify the content to cue the user to enter a terror state. For example, the user may be exploring a virtual reality environment. The ambient soundtrack may be calibrated to keep the user on edge (e.g., a soundtrack of audible, but unintelligible whispers). When the system senses that the user is most on edge, it may introduce a loud bang from behind the user. If after this loud bang is heard, the user enters a terror state then the system may modify the content to make an enemy appear proximate to the noise (e.g., to make it appear as though the enemy is sneaking up behind the user, but knocked over a broom). If however, the user did not enter the target terror state, then the system may modify the content to make the loud noise appear to come from a false alarm (e.g., a non-hostile cat knocked over a broom instead of an enemy).

### Example Use—Exposure Therapy

In some embodiments, the system may be configured to present distressing content to the user to assist the user in managing their negative reaction to the content (e.g., overcoming a phobia). In these embodiments, the content can distress the user in a step-wise fashion wherein it gradually increases the distress (e.g., a VR environment that exposes an arachnophobe to a spider). The content can start at a low intensity (e.g., the spider maintains a wide berth) and modifies the content to increase the intensity (e.g., the spider's behaviour becomes more erratic or comes closer to the user) and waits an interval to permit the user to manage their reaction to the increased intensity. If the user successfully manages their emotional response (e.g., does not reach a excess level of distress), then the content continues to increase the intensity. If the user does not manage their emotional response, then the content may return to a less intense state (e.g., the spider resumes maintaining a wide berth).

### Example Use—Drug Administration

In some embodiments, the content modification can include the delivery of drugs or medicine to induce altered consciousness states or other treatment goals. In some embodiments, the content modification can include the delivery of grounding agents to reduce the degree to which a consciousness state is altered. In some embodiments, the system can, for example, administer drugs at the opportune time to induce a state change in the user to, for example, a transformative or educational state.

In embodiments with an exit state, the drug administration can be used to permit the user to escape an intense experience. For example, if the user is using hallucinogens as part of guided therapy, then the system may be configured to deliver content to the user that challenges the user in a safe way. The system may monitor the user's distress and attempt to induce an optimum level of distress without traumatizing the user. In such embodiments, the user may start in a relaxed state and the system may be configured to probe them and bring them to a distressed state, however should the user become too distressed (e.g., experiencing lasting trauma), then the system can recognize this as an exit state and administer a sedative or other agent to quickly bring the user out of the session.

### Example Use—Pain Management

In some embodiments, systems, methods, and devices may be capable of managing pain in the user. For example, it may be configured to deliver pain-killers if the user is experiencing pain, wait an interval, and provide more if the pain is not sufficiently managed. In some embodiments, the system may be configured to apply electrical stimulus to the brain and/or a nerve of the user in lieu (or in addition to) administering drugs. Such embodiments may be helpful for chronic conditions where the user wants a certain level of lucidity that pain-killers or electrical stimulus may impede if applied in too large a dose.

The system of the present invention may be configured to control a variety of stimulus technologies to apply stimulus to the user, including transcranial magnetic stimulation (e.g., TCMS/TMS; a procedure that uses magnetic fields to stimulate nerve cells in the brain), repetitive transcranial magnetic stimulation (e.g., RTCMS/rTMS) electroconvulsive, transcranial direct current stimulation (e.g., tDCS; a form of neurostimulation which uses constant, low current delivered directly to the brain area of interest via small electrodes), electrical stimulus, and ultrasound.

Some embodiments may involve reading and stimulation of the brain to change the response of the brain. The present invention is not intended to be limited to any particular type of sensor input or stimulus type, tDCS could be substituted in most of the paradigms, for example, with the tDCS triggered when wind happens, for example. The system may stimulate your brain for you rather than the user stimulate themselves.

In the case of EEG neurofeedback, the system may read the user's brainwaves, measuring against some norm or optimum, and then rewarding the brain (through electrical, visual, audio, haptic feedback) for moving itself towards that optimum brainwave pattern.

In stimulation therapies, the system may read the state of the brain, often measure it against some norm, and then apply a stimulation modality-electric, magnetic, or ultrasound, to move it towards an optimum. The stimulation may be applied for a pre-set interval to ascertain if it successfully moves a user towards optimum.

In such embodiments, the content provided to the user may be a level of stimulus applied and it can be varied based on, for example, trigger user states, timecodes in the stimulus regime, or periodically. The system may apply variations on the level of stimulus for example for an interval to see if it induces a user state change (e.g., mitigated the pain experience).

### Example Use—Multiplayer Video Games

In some embodiments, the content provided may provide a group user experience. In some embodiments the content can be a group AR/VR experience. The content may have state modifications triggered based on the user state of one or more members of the group. The content may also periodically sample user states and modify the content for intervals to ascertain the effect of the modified content on one or more members of the group. The system may also be configured to guide the user through a narrative experience (or a game plot) based in part on the user states of one or more members of the group.

Such embodiments may be capable of providing collective group experiences that take into account the experience of one or more users to ensure the experience does not become dull or overwhelming. Such embodiments may permit the users to step into their characters in a more engaging manner.

In some embodiments, the content may be generated based in part on user inputs. For example, the system may comprise a procedural content generator that is capable of generating content based on one or more of the user states. In some embodiments, the system may be configured to offer content that is particularly impactful for one or more of the users.

### Implementation Details

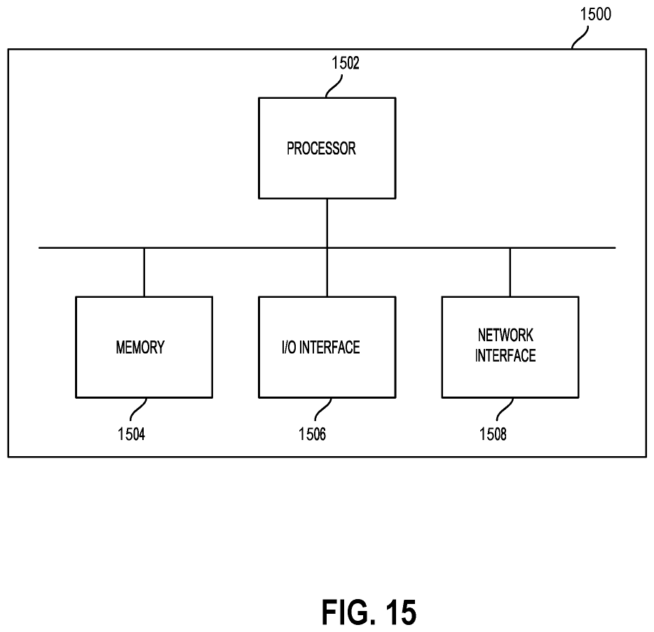

FIG. 15 is a schematic diagram of an example computing devices 12, 22, 32, or 42 suitable for implementing systems 100, 100B, 100C, 100D, 900, 1100, or 1300, in accordance with an embodiment. As depicted, computing device 1500 includes one or more processors 1502, memory 1504, one or more I/O interfaces 1506, and, can include one or more network interfaces 1508.

Each processor 1502 may be, for example, any type of general-purpose microprocessor or microcontroller, a digital signal processing (DSP) processor, an integrated circuit, a field programmable gate array (FPGA), a reconfigurable processor, a programmable read-only memory (PROM), or any combination thereof.

Memory 1504 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Memory 1504 may store code executable at processor 1502, which causes system 100, 100B, 100C, 100D, 900, 1100, or 1300 to function in manners disclosed herein. Memory 1504 includes a data storage. In some embodiments, the data storage includes a secure datastore. In some embodiments, the data storage stores received data sets, such as textual data, image data, or other types of data.

Each I/O interface 1506 enables computing device 1500 to interconnect with one or more input devices, such as a keyboard, mouse, camera, touch screen and a microphone, or with one or more output devices such as a display screen and a speaker.

Each network interface 1508 enables computing device 1500 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WIMAX), SS7 signaling network, fixed line, local area network, wide area network, and others, including any combination of these.

The methods disclosed herein may be implemented using a system 100, 100B, 100C, 100D, 900, 1100, or 1300 that includes multiple computing devices 1500. The computing devices 1500 may be the same or different types of devices.

Each computing devices may be connected in various ways including directly coupled, indirectly coupled via a network, and distributed over a wide geographic area and connected via a network (which may be referred to as “cloud computing”).

For example, and without limitation, each computing device 1500 may be a server, network appliance, set-top box, embedded device, computer expansion module, personal computer, laptop, personal data assistant, cellular telephone, smartphone device, UMPC tablets, video display terminal, gaming console, electronic reading device, and wireless hypermedia device or any other computing device capable of being configured to carry out the methods described herein.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references were made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The foregoing discussion provides many example embodiments. Although each embodiment represents a single combination of inventive elements, other examples may include all possible combinations of the disclosed elements. Thus if one embodiment comprises elements A, B, and C, and a second embodiment comprises elements B and D, other remaining combinations of A, B, C, or D, may also be used.

The term “connected” or “coupled to” may include both direct coupling (in which two elements that are coupled to each other contact each other) and indirect coupling (in which at least one additional element is located between the two elements).

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements. The embodiments described herein are directed to electronic machines and methods implemented by electronic machines adapted for processing and transforming electromagnetic signals which represent various types of information. The embodiments described herein pervasively and integrally relate to machines, and their uses; and the embodiments described herein have no meaning or practical applicability outside their use with computer hardware, machines, and various hardware components. Substituting the physical hardware particularly configured to implement various acts for non-physical hardware, using mental steps for example, may substantially affect the way the embodiments work. Such computer hardware limitations are clearly essential elements of the embodiments described herein, and they cannot be omitted or substituted for mental means without having a material effect on the operation and structure of the embodiments described herein. The computer hardware is essential to implement the various embodiments described herein and is not merely used to perform steps expeditiously and in an efficient manner.

The embodiments and examples described herein are illustrative and non-limiting. Practical implementation of the features may incorporate a combination of some or all of the aspects, and features described herein should not be taken as indications of future or existing product plans. Applicant partakes in both foundational and applied research, and in some cases, the features described are developed on an exploratory basis.

Although the embodiments have been described in detail, it should be understood that various changes, substitutions and alterations can be made herein without departing from the scope as defined by the appended claims.

Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. A computer system for achieving a target user state by modifying content elements provided to at least one user, the system comprising:
at least one computing device in communication with at least one bio-signal sensor and at least one user effector;
the at least one bio-signal sensor configured to measure bio-signals of at least one user;
the at least one user effector configured to provide content to the at least one user, wherein the content comprises one or more content elements;
the at least one computing device configured to:
provide the content to the at least one user via the at least one user effector;
compute a difference between the user state of the at least one user before an interval and the target user state using the bio-signals of the at least one user;
modify one or more of the content elements provided to the at least one user during the interval based on the difference between the user state of the at least one user before the interval and the target user state;
compute a difference between the user state of the at least one user after the interval and the target user state using the bio-signals of the at least one user;
modify one or more of the content elements provided to the at least one user after the interval based on the difference between the user state of the at least one user after the interval and the target user state.

2. The system of claim 1, wherein:
the compute a difference between the user state of the at least one user before an interval and the target user state comprises determining that a trigger user state has been achieved using the bio-signals of the at least one user.

3. The system of claim 1, wherein:
the at least one user effector is configured to provide content to a plurality of users;
the user state is based on the bio-signals of each user of the plurality of users.

4. The system of claim 1, wherein the user state is determined based in part on a prediction model.

5. The system of claim 4, further comprising:
a server configured to:
store the prediction model; and
provide the prediction model to the at least one computing device;
and the at least one computing device is configured to update the prediction model based on the difference between the user state of the at least one user after the interval and the target user state.

6. The system of claim 4 wherein the prediction model comprises a neural network.

7. The system of claim 4 wherein the prediction model is based in part on a user profile.

8. The system of claim 4 wherein the prediction model is based in part on data from one or more other users.

9. The system of claim 8 wherein the one or more other users share a characteristic with the at least one user.

10. The system of claim 1 wherein the interval is based in part on a current user state of the at least one user.

11. The system of claim 1 wherein the interval is based in part the content.

12. The system of claim 1 wherein the interval is based in part on user input.

13. The system of claim 1 wherein the target user state is based in part on the content.

14. The system of claim 1 wherein the target user state is based in part on input.

15. The system of claim 2 wherein the trigger user state is based in part on the content.

16. The system of claim 2 wherein the trigger user state is based in part on input.

17. The system of claim 1 wherein the modify the one or more of the content elements is based in part on user input.

18. The system of claim 2 wherein the at least one computing device is further configured to:
determine a first user state of the at least one user using the bio-signals of the at least one user;
apply a probe modification to one or more of the content elements provided to the at least one user;
compute a difference between the first user state of the at least one user and the user state of the at least one user after a probe interval using the bio-signals of the at least one user;
update at least one of the target user state and the trigger user state based on the difference between the first user state and the user state after the probe interval.

19. The system of claim 2, wherein the at least one computing device is further configured to:
determine a first user state of the at least one user using the bio-signals of the at least one user before a probe interval;
compute a difference between the first user state of the at least one user before the probe interval and a user state of the at least one user after the probe interval using the bio-signals of the at least one user;
update at least one of the target user state and the trigger user state based on the difference between the first user state and the user state after the probe interval.

20. The system of claim 1 wherein the computing device is further configured to:
compute a difference between the user state of the at least one user during the interval and an exit user state using the bio-signals of the at least one user;
modify one or more of the content elements provided to the at least one user based on the difference between the user state of the at least one user and the exit user state.

21. The system of claim 1, wherein the bio-signal sensor comprises at least one of EEG, EOG, EKG, EMG, PPG, heart rate, breath, sweat, gyroscopic, accelerometer, magnetometer, IMU, movement, vibration, sound, pulse wave amplitude, fNIRS, temperature, pressure, and electrodermal conductance sensors.

22. The system of claim 1, wherein the at least one user effector comprises at least one of earphones, speakers, a display, a scent diffuser, a heater, a climate controller, a drug infuser or administrator, an electric stimulator, a medical device, a system to effect physical or chemical changes in the body, restraints, a mechanical device, a vibrotactile device, and a light.

23. The system of claim 1, further comprising:
one or more auxiliary effectors configured to provide stimulus to the at least one user; and
wherein the computing device is further configured to modify the stimulus provided to the at least one user by the auxiliary effector.

24. The system of claim 1, wherein the modify one or more of the content elements comprises transitioning between one or more content samples.

25. The system of claim 1, wherein the modify one or more of the content elements comprises pausing one or more of the content elements.

26. The system of claim 1, wherein the modify one or more of the content elements comprises pausing one or more of the content elements at time codes associated with natural breaks in the one or more content elements.

27. The system of claim 1, wherein the computing device is further configured to adjust the interval based on natural breaks in the one or more of the content elements.

28. The system of claim 1, wherein:
the content comprises at least a first and a second time-coded content sample;
the modify one or more of the content elements comprises transitioning between a first defined time code of the first time-coded content sample to a second defined time code of the second time-coded content sample.

29. The system of claim 28, wherein the first defined time code is based on natural pauses in the first time-coded content sample and the second defined time code is based on natural pauses in the second time-coded content sample.

30. The system of claim 28, wherein the second time-coded content sample is selected from a plurality of time-coded content samples based on at least on of the first time-coded content sample.

31. The system of claim 30, wherein the selection of the second time-coded content sample is based in part on a prediction model.

32. The system of claim 1, wherein:
the content comprises time-coded content; and
the modify one or more of the content elements is based in part on a current time code in the time-coded content.

33. The system of claim 1, wherein the user state comprises a brain state.

34. The system of claim 1, wherein the content elements have modifications applied at a specific change profile.

35. The system of claim 2, wherein the trigger user state comprises reaching a time code in the content.

36. A method for achieving a target user state by modifying content elements provided to at least one user, the method comprising:
receiving bio-signals of at least one user;
providing content to the at least one user, the content comprising one or more content elements;
computing a difference between a user state of the at least one user before an interval and the target user state using the bio-signals of the at least one user;
modifying one or more of the content elements provided to the at least one user during the interval based on the difference between the user state of the at least one user before the interval and the target user state;
computing a difference between the user state of the at least one user after an interval and the target user state using the bio-signals of the at least one user;
modifying one or more of the content elements provided to the at least one user after the interval based on the difference between the user state of the at least one user after the interval and the target user state.

37. The method of claim 36, wherein:
computing a difference between the user state of the at least one user before an interval and the target user state comprises determining that a trigger user state has been achieved using the bio-signals of the at least one user.

38. The method of claim 36, wherein:
the providing content to at least one user comprises providing content to a plurality of users;
the user state is based on the bio-signals of each user of the plurality of users.

39. The method of claim 36, wherein the user state is determined based in part on a prediction model.

40. The method of claim 39, further comprising:
updating the prediction model based on the difference between the user state of the at least one user after the interval and the target user state.

41. The method of claim 39 wherein the prediction model comprises a neural network.

42. The method of claim 39 wherein the prediction model is based in part on a user profile.

43. The method of claim 39 wherein the prediction model is based in part on data from one or more other users.

44. The method of claim 43 wherein the one or more other users share a characteristic with the at least one user.

45. The method of claim 36 wherein the interval is based in part on a current user state of the at least one user.

46. The method of claim 36 wherein the interval is based in part the content.

47. The method of claim 36 wherein the interval is based in part on user input.

48. The method of claim 36 wherein the target user state is based in part on the content.

49. The method of claim 36 wherein the target user state is based in part on input.

50. The method of claim 37 wherein the trigger user state is based in part on content.

51. The method of claim 37 wherein the trigger user state is based in part on input.

52. The method of claim 36 wherein modifying the one or more of the content elements is based in part on user input.

53. The method of claim 37 further comprising:
determining a first user state of the at least one user using the bio-signals of the at least one user;
applying a probe modification to one or more of the content elements provided to the at least one user;
computing a difference between the first user state of the at least one user and the user state of the at least one user after a probe interval using the bio-signals of the at least one user;
updating at least one of the target user state and the trigger user state based on the difference between the first user state and the user state after the probe interval.

54. The method of claim 37, further comprising:
determining a first user state of the at least one user using the bio-signals of the at least one user before a probe interval;
computing a difference between the first user state of the at least one user before the probe interval and a user state of the at least one user after the probe interval using the bio-signals of the at least one user;
updating at least one of the target user state and the trigger user state based on the difference between the first user state and the user state after the probe interval.

55. The method of claim 36 wherein the method further comprises:
computing a difference between the user state of the at least one user during the interval and an exit user state using the bio-signals of the at least one user;
modifying one or more of the content elements provided to the at least one user based on the difference between the user state of the at least one user and the exit user state;

56. The method of claim 36, further comprising modifying auxiliary stimulus provided to the at least one user.

57. The method of claim 36, wherein the modifying one or more of the content elements comprises transitioning between one or more content samples.

58. The method of claim 36, wherein the modifying one or more of the content elements comprises pausing one or more of the content elements.

59. The method of claim 36, wherein the modifying one or more of the content elements comprises pausing one or more of the content elements at time codes associated with natural breaks in the one or more content elements.

60. The method of claim 36, further comprising adjusting the interval based on natural breaks in the one or more of the content elements.

61. The method of claim 36, wherein:
the content comprises at least a first and a second time-coded content sample;
the modifying one or more of the content elements comprises transitioning between a first defined time-code of the first time-coded content sample to a second defined time-code of the second time-coded content sample.

62. The method of claim 61, wherein the first defined time code is based on natural pauses in the first time-coded content sample and the second defined time code is based on natural pauses in the second time-coded content sample.

63. The method of claim 61, wherein the second time-coded content sample is selected from a plurality of time-coded content samples based on at least on of the first time-coded content sample.

64. The method of claim 63, wherein the selection of the second time-coded content sample is based in part on a prediction model.

65. The method of claim 36, wherein:
the content comprises time-coded content; and
the modifying one or more of the content elements is based in part on a current time code in the time-coded content.

66. The method of claim 36, wherein the user state comprises a brain state.

67. The method of claim 36, wherein the content elements have modifications applied at a specific change profile.

68. The method of claim 37, wherein the trigger user state comprises reaching a time code in the content.

69. The use of time-coded content to induce a change in state of at least one user by presenting the time-coded content to the at least one user and using a bio-signal sensor, the time-coded content comprising:
one or more content elements;
one or more content modification processes;
the content modification processes comprising a modification, a trigger, a target user state, and at least one interval;
the content modification processes configured to:
initiate the modification on detecting that the trigger is satisfied;
modify one or more of the content elements based in part on the modification during the at least one interval;
modify one or more of the content elements based on a difference between a user state of the at least one user after the at least one interval, the target user state, and the modification.

70. The use of claim 69, wherein:
the trigger comprises a trigger user state that the at least one user must satisfy; and
the modify one or more of the content elements based in part on the modification comprises modifying the one or more content element based in part on the user state.

71. The use of claim 69, wherein:
the trigger comprises a time code in the content; and
the modify one or more of the content elements based in part on the modification comprises modifying one or more of the content elements at or after the time code.

72. The use of claim 69, wherein:
the bio-signals of the at least one user comprise bio-signals of a plurality of users; and
the user state is based on each user of the plurality of users.

73. The use of claim 69, wherein the user state is determined based in part on a prediction model.

74. The use of claim 73, further comprising:
a server configured to:
store the prediction model; and
provide the prediction model to the at least one computing device;
and the at least one computing device is configured to update the prediction model based on the difference between the user state of the at least one user after the at least one interval and the target user state.

75. The use of claim 73 wherein the prediction model comprises a neural network.

76. The use of claim 73 wherein the prediction model is based in part on a user profile.

77. The use of claim 73 wherein the prediction model is based in part on data from one or more other users.

78. The use of claim 77 wherein the one or more other users share a characteristic with the at least one user.

79. The use of claim 69 wherein the at least one interval is based in part on a current user state of the at least one user.

80. The use of claim 69 wherein the at least one interval is based in part on the content.

81. The use of claim 69 wherein the at least one interval is based in part on user input.

82. The use of claim 69 wherein the target user state is based in part on the content.

83. The use of claim 69 wherein the target user state is based in part on input.

84. The use of claim 70 wherein the trigger user state is based in part on the content.

85. The use of claim 70 wherein the trigger user state is based in part on input.

86. The use of claim 69 wherein modifying the one or more of the content elements is based in part on user input.

87. The use of claim 69 wherein at least one content modification process is configured to:
determine a first user state of the at least one user using the bio-signals of the at least one user;
apply a probe modification to one or more of the content elements provided to the at least one user;
compute a difference between the first user state of the at least one user and the user state of the at least one user after a probe interval using the bio-signals of the at least one user;
update at least one of the modification, the target user state, the trigger, and the at least one interval of one or more content modification processes based on a difference between the first user state and the user state of the at least one user after the probe interval.

88. The use of claim 70, wherein at least one content modification process is configured to:
determine a first user state of the at least one user using the bio-signals of the at least one user before a probe interval;
compute a difference between the first user state of the at least one user before the probe interval and a user state of the at least one user after the probe interval using the bio-signals of the at least one user;
update at least one of the target user state and the trigger user state based on the difference between the first user state and the user state after the probe interval.

89. The use of claim 69 wherein the content modification process further comprises an exit user state and is further configured to:
modify one or more of the content elements provided to the at least one user based on the difference between the user state of the at least one user during the at least one interval and the exit user state;

90. The use of claim 69, wherein the bio-signal sensor comprises at least one of EEG, EOG, EKG, EMG, PPG, heart rate, breath, sweat, gyroscopic, accelerometer, magnetometer, IMU, movement, vibration, sound, pulse wave amplitude, fNIRS, temperature, pressure, and electrodermal conductance sensors.

91. The use of claim 69, wherein the at least one user effector comprises at least one of earphones, speakers, a display, a scent diffuser, a heater, a climate controller, a drug infuser or administrator, an electric stimulator, a medical device, a system to effect physical or chemical changes in the body, restraints, a mechanical device, a vibrotactile device, and a light.

92. The use of claim 69, the content modification process is further configured to modify auxiliary stimulus provided to the at least one user.

93. The use of claim 69, wherein the modify one or more of the content elements comprises transitioning between one or more content samples.

94. The use of claim 69, wherein the modify one or more of the content elements comprises pausing one or more of the content elements.

95. The use of claim 69, wherein the modify one or more of the content elements comprises pausing one or more of the content elements at time codes associated with natural breaks in the one or more content elements.

96. The use of claim 69, wherein the content modification process adjusts the interval based on natural breaks in the one or more of the content elements.

97. The use of claim 69, wherein:
the time-coded content comprises at least a first and a second time-coded content sample;
the modify one or more of the content elements comprises transitioning between a first defined time-code of the first time-coded content sample to a second defined time-code of the second time-coded content sample.

98. The use of claim 97, wherein the first defined time code is based on natural pauses in the first time-coded content sample and the second defined time code is based on natural pauses in the second time-coded content sample.

99. The use of claim 97, wherein the second time-coded content sample is selected from a plurality of time-coded content samples based on at least on of the first time-coded content sample.

100. The use of claim 99, wherein the selection of the second time-coded content sample is based in part on a prediction model.

101. The use of claim 69, wherein the user state comprises a brain state.

102. The use of claim 69, wherein the content elements have modifications applied at a specific change profile.

103. The use of claim 70, wherein the trigger user state comprises reaching a time code in the content.

104. A computer system to develop time-coded content for achieving an ultimate user state by modifying content elements provided to at least one user, the system comprising:
at least one computing device in communication with at least one bio-signal sensor and at least one user effector;
the at least one bio-signal sensor configured to measure bio-signals of at least one user;
the at least one user effector configured to provide time-coded content to the at least one user, wherein the time-coded content comprises one or more content elements;
the at least one computing device configured to:
provide the time-coded content to the at least one user via the at least one user effector;
determine an initial user state of the at least one user at a time code;
modify one or more of the content elements provided to the at least one user;
determine a final user state of the at least one user after a test interval;
update the time-coded content to provide a content modification process comprising, a target user state, an interval, a modification, and at least one of a time code and a trigger user state, wherein the trigger user state is based on the initial user state, the target user state is based on the final user state, the interval is based on the test interval, and the modification and the time code are based on the modify one or more of the content elements.

105. The system of claim 104 wherein the at least one computing device is further configured to:
determine another initial user state of the at least one user at another time code, wherein the another initial user state is determined with or after the final user state;
modify one or more of the content elements provided to the at least one user;
determine another final user state of the at least one user after another test interval;
update the time-coded content to provide at least one more content modification process comprising a target user state, an interval, a modification, and at least one of a time code and a trigger user state, wherein the trigger user state is based on the another initial user state, the target user is based on the another final user state, the interval is based on the another test interval, and the modification and the time code are based on the modify one or more of the content elements.

106. The system of claim 104, wherein:
the time code comprises at least one of a regular, a random, a pre-defined, an algorithmically defined, a user defined, and a triggered time code.

107. The system of claim 104, wherein:
the interval comprises at least one of a regular, a random, a pre-defined, a user defined, and an algorithmically defined interval.

108. The system of claim 104, wherein:
the modification comprises at least one of a random, a pre-defined, a user defined, and an algorithmically defined modification.

109. The system of claim 104, wherein the time-coded content is pre-processed to extract one or more content elements.

110. The system of claim 104, wherein:
the at least one user effector is configured to provide time-coded content to a plurality of users;
the user state is based on the bio-signals of each user of the plurality of users.

111. The system of claim 104 wherein the content modification processes are based in part on a user profile.

112. The system of claim 104 wherein the interval is based in part on a current user state of the at least one user.

113. The system of claim 104 wherein the content modification processes further comprise:
an exit user state based on the final user state, the ultimate user state, and the modify one or more of the content elements.

114. The system of claim 104, wherein the bio-signal sensor comprises at least one of EEG, EOG, EKG, EMG, PPG, heart rate, breath, sweat, gyroscopic, accelerometer, magnetometer, IMU, movement, vibration, sound, pulse wave amplitude, fNIRS, temperature, pressure, and electrodermal conductance sensors.

115. The system of claim 104, wherein the at least one user effector comprises at least one of earphones, speakers, a display, a scent diffuser, a heater, a climate controller, a drug infuser or administrator, an electric stimulator, a medical device, a system to effect physical or chemical changes in the body, restraints, a mechanical device, a vibrotactile device, and a light.

116. The system of claim 104, further comprising:
one or more auxiliary effectors configured to provide stimulus to the at least one user; and
wherein the computing device is further configured to modify the stimulus provided to the at least one user by the auxiliary effector.

117. The system of claim 104, wherein the modify one or more of the content elements comprises transitioning between one or more content samples.

118. The system of claim 104, wherein the modify one or more of the content elements comprises pausing one or more of the content elements.

119. The system of claim 104, wherein the modify one or more of the content elements comprises pausing one or more of the content elements at time codes associated with natural breaks in the one or more content elements.

120. The system of claim 104, wherein the computing device is further configured to adjust the interval based on natural breaks in the one or more of the content elements.

121. The system of claim 104, wherein:
the time-coded content comprises at least a first and a second time-coded content sample;
the modify one or more of the content elements comprises transitioning between a first defined time code of the first time-coded content sample to a second defined time code of the second time-coded content sample.

122. The system of claim 121, wherein the first defined time code is based on natural pauses in the first time-coded content sample and the second defined time code is based on natural pauses in the second time-coded content sample.

123. The system of claim 121, wherein the second time-coded content sample is selected from a plurality of time-coded content samples based on at least on of the first time-coded content sample.

124. The system of claim 104, wherein the user state comprises a brain state.

125. The system of claim 104, wherein the content elements have modifications applied at a specific change profile.

126. A method to develop time-coded content for achieving an ultimate user state by modifying content elements provided to at least one user, the method comprising:
providing the time-coded content to the at least one user, the time-coded content comprising content elements;
determining an initial user state of the at least one user at a time code using bio-signals of the at least one user;
modifying one or more of the content elements provided to the at least one user;
determining a final user state of the at least one user after a test interval;
updating the time-coded content to provide a content modification process comprising a target user state, an interval, a modification, and at least one of a time code and a trigger user state, wherein the trigger user state is based on the initial user state, the target user state is based on the final user state, the interval is based on the test interval, and the modification and the time code are based on the modifying one or more of the content elements.

127. The method of claim 126 further comprising:
determining another initial user state of the at least one user at another time code, wherein the another initial user state is determined with or after the final user state;
modifying one or more of the content elements provided to the at least one user;
determining another final user state of the at least one user after another test interval;
updating the time-coded content to provide at least one more content modification process comprising a target user state, an interval, a modification, and at least one of a time code and a trigger user state, wherein the trigger user state is based on the another initial user state, the target user state is based on the another final user state, the interval is based on the another test interval, and the modification and the time code are based on the modifying one or more of the content elements.

128. The method of claim 126, wherein:
the time code comprises at least one of a regular, a random, a pre-defined, an algorithmically defined, a user defined, and a triggered time code.

129. The method of claim 126, wherein:
the interval comprises at least one of a regular, a random, a pre-defined, a user defined, and an algorithmically defined interval.

130. The method of claim 126, wherein:
the modification comprises at least one of a random, a pre-defined, a user defined, and an algorithmically defined modifications.

131. The method of claim 126, wherein the time-coded content is pre-processed to extract one or more content elements.

132. The method of claim 126, wherein:
the at least one user comprises a plurality of users;
the user state is based on the bio-signals of each user of the plurality of users.

133. The method of claim 126, wherein the content modification processes are based in part on a user profile.

134. The method of claim 126, wherein the interval is based in part on a current user state of the at least one user.

135. The method of claim 126, wherein the content modification processes further comprise:
an exit user state based on the final user state, the ultimate user state, and the modify one or more of the content elements.

136. The method of claim 126, further comprising modifying auxiliary stimulus provided to the at least one user.

137. The method of claim 126, wherein the modifying one or more of the content elements comprises transitioning between one or more content samples.

138. The method of claim 126, wherein the modifying one or more of the content elements comprises pausing one or more of the content elements.

139. The method of claim 126, wherein the modify one or more of the content elements comprises pausing one or more of the content elements at time codes associated with natural breaks in the one or more content elements.

140. The method of claim 126, wherein the computing device is further configured to adjust the interval based on natural breaks in the one or more of the content elements.

141. The method of claim 126, wherein:
the time-coded content comprises at least a first and a second time-coded content sample;
the modifying one or more of the content elements comprises transitioning between a first defined time code of the first time-coded content sample to a second defined time code of the second time-coded content sample.

142. The method of claim 141, wherein the first defined time code is based on natural pauses in the first time-coded content sample and the second defined time code is based on natural pauses in the second time-coded content sample.

143. The method of claim 141, wherein the second time-coded content sample is selected from a plurality of time-coded content samples based on at least on of the first time-coded content sample.

144. The method of claim 126, wherein the user state comprises a brain state.

145. The method of claim 126, wherein the content elements have modifications applied at a specific change profile.

146. A computer system to detect a user state of at least one user, the system comprising:
at least one computing device in communication with at least one bio-signal sensor, and at least one other signal sensor;
the at least one bio-signal sensor configured to measure bio-signals of at least one user;
the at least one other signal sensor configured to measure other signals of the at least one user;
the at least one computing device configured to:
measure the bio-signals of the at least one user;
measure the other signals of the at least one user;
determine a user state of the at least one user using the measured bio-signals and a prediction model;
update the prediction model with the determined user state and the measured other signals of the at least one user;
determine the user state of the at least one user using the measured other signals and the updated prediction model.

147. The system of claim 146 wherein the system is further configured to perform an action based on the user state determined using the measured other signals and the updated prediction model.

148. The system of claim 146, further comprising:
a server configured to:
store the prediction model; and
provide the prediction model to the at least one computing device;
and the at least one computing device is configured to update the prediction model on the server.

149. The system of claim 146 wherein the prediction model comprises a neural network.

150. The system of claim 146 wherein the other signals comprises at least one of a typing speed, a temperature preference, ambient noise, a user objective, a location, ambient temperature, an activity type, a social context, a user preferences, self-reported user data, dietary information, exercise level, activities, a dream journal, emotional reactivity, behavioural data, content consumed, contextual signals, search history, and social media activity.

151. The system of claim 146 wherein the other signals comprises bio-signals or behaviours of other individuals.

152. The system of claim 146 wherein the prediction model is based in part on a user profile.

153. The system of claim 146 wherein the prediction model is based in part on data from one or more other users.

154. The system of claim 153 wherein the one or more other users share a characteristic with the at least one user.

155. The system of claim 146, wherein the bio-signal sensor comprises at least one of EEG, EOG, EKG, EMG, PPG, heart rate, breath, sweat, gyroscopic, accelerometer, magnetometer, IMU, movement, vibration, sound, pulse wave amplitude, fNIRS, temperature, pressure, and electrodermal conductance sensors.

156. The system of claim 146, wherein the user state comprises a brain state.

157. A method to detect a user state of at least one user, the method comprising:
measuring bio-signals of at least one user;
measuring other signals of the at least one user;
determining a user state of the at least one user using the measured bio-signals and a prediction model;
updating the prediction model with the determined user state and the measured other signals of the at least one user;
determining the user state of the at least one user using the measured other signals and the updated prediction model.

158. The method of claim 157 further comprising performing an action based on the user state determined using the measured other signals and the updated prediction model.

159. The method of claim 157 wherein the prediction model comprises a neural network.

160. The method of claim 157 wherein the other signals comprises at least one of a typing speed, a temperature preference, ambient noise, a user objective, a location, ambient temperature, an activity type, a social context, a user preferences, self-reported user data, dietary information, exercise level, activities, dream journals, emotional reactivity, behavioural data, content consumed, contextual signals, search history, and social media activity.

161. The method of claim 157 wherein the other signals comprises bio-signals or behaviours of other individuals.

162. The method of claim 157 wherein the prediction model is based in part on a user profile.

163. The method of claim 157 wherein the prediction model is based in part on data from one or more other users.

164. The method of claim 163 wherein the one or more other users share a characteristic with the at least one user.

165. The method of claim 157, wherein the user state comprises a brain state.

166. A computer system to map user states, the system comprising:
at least one computing device in communication with at least one bio-signal sensor and at least one user effector;
the at least one bio-signal sensor configured to measure bio-signals of at least one user;
the at least one user effector configured to provide stimulus to the at least one user;
the at least one computing device configured to:
determine an initial user state of at least one user using the at least one bio-signal sensor;
provide stimulus to the at least one user;
determine a final user state of at least one user using the at least one bio-signal sensor;
update a user state map using the stimulus, initial user state, final user state.

167. The system of 166, wherein the user state map is updated using a time code at which the stimulus was provided to the at least one user.

168. The system of 166, wherein the computing device is further configured to:
receive user input on the initial user state or the final user state that describes the state.

169. The system of 166, wherein the computing device is further configured to:
provide stimulus to the at least one user that is predicted to direct the at least one user into desirable user states.

170. The system of 166, wherein the determine the final user state comprises determining the final user state after an interval.

171. The system of 166, wherein:
the stimulus comprises modification of content presented to the at least one user; and
the update a user state map comprises generating content modification process comprising:
a trigger user state based on the initial user state,
a target user state based on the final user state, and
a modification based on the modification of content presented to the at least one user.

172. The system of 171, wherein the computing device is further configured to:
induce the target user state by initiating the content modification process when the at least one user achieves the trigger user state.

173. The system of claim 171 wherein the user state map is associated with a user profile of the at least one user and the system is further configured to apply the content modification process to other content when the user achieves the trigger user state.

174. A method to map user states, the method comprising:
determining an initial user state of at least one user;
providing stimulus to the at least one user;
determining a final user state of at least one user;
updating a user state map using the stimulus, initial user state, final user state.

175. The method of 0, wherein updating the user state map comprises updating the user state map using a time code at which the stimulus was provided to the at least one user.

176. The method of 0, the method further comprising:
receiving user input on the initial user state or the final user state that describes the state.

177. The method of 0, the method further comprising:
providing stimulus to the at least one user that is predicted to direct the at least one user into desirable user states.

178. The method of 0, wherein the determining the final user state comprises determining the final user state after an interval.

179. The method of 0, wherein:
the stimulus comprises modification of content presented to the at least one user; and
the updating a user state map comprises generating content modification process comprising:
a trigger user state based on the initial user state,
a target user state based on the final user state, and
a modification based on the modification of content presented to the at least one user.

180. The method of 179, further comprising:
inducing the target user state by initiating the content modification process when the at least one user achieves the trigger user state.

181. The method of claim 179 further comprising:
associating the user state map with a user profile of the at least one user; and
applying the content modification process to other content when the user achieves the trigger user state.

182. A non-transient computer readable medium containing program instructions for causing a computer to perform the method of any of claims 36 to 68, 126 to 145, 157 to 165, and 0 to 181.

183. A hardware processor configured to assist in achieving a target user state by processing bio-signals of at least one user captured by at least one bio-signal sensor and triggering at least one user effector to modify one or more of content elements, the hardware processor executing code stored in non-transitory memory to implement operations described in the description or drawings.

184. A method to assist in achieving a target user state by processing, using a hardware processor, bio-signals of at least one user captured by at least one bio-signal sensor and triggering at least one user effector to modify one or more of content elements, the method comprising steps described in the description or drawings.
